(19) United States
(12) Patent Application Publication
(10) Pub. No.: US 2026/0007643 A1
(43) Pub. Date: Jan. 8, 2026

(54) HIGHLY SOLUBLE FORMULATIONS OF HARMINE

(71) Applicant: Reconnect Labs AG, Winterthur (CH)

(72) Inventors: Dario Dornbierer, Zurich (CH); Milan Scheidegger, Winterthur (CH); Davor Kosanic, Lausanne (CH); Daniel Claussen, Engelberg (CH); Michael Kometer, Weggis (CH); Robin Von Rotz, Zürich (CH); Robert Hett, Muttenz (CH); Maxim Puchkov, Pfeffingen (CH); Roland Haag, Lörrach (DE)

(21) Appl. No.: 18/992,702

(22) PCT Filed: Jul. 27, 2023
(86) PCT No.: PCT/EP2023/070938

§ 371 Date: Jan. 9, 2025

(30) Foreign Application Priority Data
Jul. 27, 2022 (EP) 22187342.5
Jul. 27, 2022 (EP) 22187349.0

(51) Int. Cl. A61K 31/437; A61K 9/00; A61K 9/20; A61K 31/485; A61K 47/12; A61K 47/26

(52) U.S. Cl. CPC A61K 31/437; A61K 9/006; A61K 9/2018; A61K 31/485; A61K 47/12; A61K 47/26

(57) ABSTRACT

The present invention relates to a composition comprising harmine and (i) an uronic acid or (ii) a carboxylic acid and a monosaccharide, to a salt of harmine and uronic acid, to a kit of parts comprising (a) the composition or the salt of the invention and a pharmaceutically acceptable carrier and (b) DMT or a pharmaceutically acceptable salt thereof and a pharmaceutically acceptable carrier, to a pharmaceutical composition comprising the composition or the salt of the invention and a pharmaceutically acceptable carrier. The compositions, the salts, the kits of parts and the pharmaceutical compositions of the present invention are particularly useful in the treatment of psychiatric, psychosomatic or somatic disorders.

### FIELD OF THE INVENTION

The present invention relates to a composition comprising harmine and (i) an uronic acid or (ii) a carboxylic acid and a monosaccharide, to a salt of harmine and uronic acid, to a kit of parts comprising (a) the composition or the salt of the invention and a pharmaceutically acceptable carrier and (b) DMT or a pharmaceutically acceptable salt thereof and a pharmaceutically acceptable carrier, to a pharmaceutical composition comprising the composition or the salt of the invention and a pharmaceutically acceptable carrier. The compositions, the salts, the kits of parts and the pharmaceutical compositions of the present invention are particularly useful in the treatment of psychiatric, psychosomatic or somatic disorders.

### BACKGROUND OF THE INVENTION

Affective spectrum disorders are widespread in society and are significant contributors to the current economic burden in health care, reaching double-digit billion CHF amounts in Switzerland and orders of magnitude more worldwide. Along the affective spectrum the most prevalent mood disorders include depression (major depressive disorder, dysthymia, double depression, seasonal affective disorder, burnout, postpartum depression, premenstrual dysphoric disorder) and bipolar disorders (characterized by periods of depression and hypomania/mania). Despite high prevalence, most of the available therapies show suboptimal efficacy and are currently prescribed in a lengthy trial and error approach for weeks or months to see clinical benefit. Still fewer than 50% of all patients with depression show full remission with optimized standard treatment, including trials on numerous medications. Thus, there is an urgent need for novel mental health therapies with more rapid and sustainable therapeutic effects.

Recently, a novel class of rapid-acting antidepressant psychotropic compounds such as ketamine, psilocybin, and LSD was discovered to alleviate symptoms of anxiety and depression. Repeated administration of ketamine was shown to sustain antidepressant effects, but puts patients at risk due to its addictive potential. Moreover, there are major shortcomings of using compounds such as LSD for clinical purposes due to its long duration of action (10-12 hours). Additionally, both LSD and psilocybin show rapid tolerance at serotonergic receptors (Nichols 2016), which makes them less suited for repeated dosing regimens.

In contrast, the traditional indigenous plant concoction commonly made from Banisteriopsis caapi and Psychotria viridis or Diplopterys cabrerana, called ayahuasca, known as psychedelic agent, is increasingly recognized due to its beneficial effects on physical and mental health, making it a promising candidate for therapeutic use (Domínguez-Clavé et al. 2016). Herein, psychedelic agent refers to an agent that can cause an altered state of consciousness in a subject that uses it. Altered state of consciousness refers to any condition different from a normal waking state, and may include, but is not limited to, experiencing cognitive or perceptual alterations (e.g. hallucinations), intense emotions, or day-dreaming. Ayahuasca has been suggested to exhibit positive effects in patients with psychological, somatic, and psychosomatic illnesses and has been used for centuries in natural medicine in Latin American regions (Frecska et al. 2016). In small pilot studies, ayahuasca shows rapid and more sustained antidepressant properties in depressed patients (Osório et al. 2015; Palhano-Fontes et al. 2018; Santos et al. 2016), compared to the transient antidepressant effects of ketamine, where a considerable number of patients relapse within 7 days of treatment (Sanacora et al. 2016). While the mechanism of such action is not known, the potentially therapeutic effect of ayahuasca is hypothesized to rely on its ability of resetting neuronal circuits underlying maladaptive neurobehavioral states.

Ayahuasca concoction comprises a mixture of N,N-dimethyltryptamine (DMT) and beta-carbolines (e.g. harmine, harmaline, tetrahydroharmine, among others.). Ayahuasca is a) non-toxic, b) has a low addictive abuse potential, c) does not produce tolerance, and d) shows an antidepressant potential (Domínguez-Clavé et al. 2016; Barbosa et al. 2012). In order for DMT to become bioavailable, peroral formulations usually contain plant-based sources of DMT (e.g. from Psychotria viridis) combined with β-carbolines (e.g. from Banisteriopsis caapi) that act as selective reversible monoamine oxidase A (MAO-A) inhibitors to prevent degradation of DMT in the body (Callaway et al. 1996). DMT is a structural analogue of serotonin and is widely found in nature, including plants, mammalian organisms, human brains and body fluids (Barker 2018).

Although ayahuasca ingestion is considered safe (Barbosa et al. 2012), it brings along a number of undesired side effects (e.g. nausea, vomiting, diarrhea, hallucinations), compromising its clinical utility. Most of its side effects can be attributed to suboptimal pharmacokinetic/-dynamic properties due to the random admixture of plant material (with unknown or adverse toxicity), and variability in alkaloid content—precluding its use as a standardized prescription medicine in the clinical context. Moreover, upon peroral administration of ayahuasca, DMT is readily absorbed into the bloodstream and causes rapid changes in the consumer's perception, with potentially distressing side effects (Riba et al. 2003).

An alternative to ayahuasca that solves the problems of side effects outlined above is pharmahuasca, also known as synthetic ayahuasca. According to the disclosure of the document DE102016014603A1, the term pharmahuasca or synthetic ayahuasca relates to combinations, compositions, mixtures and preparations comprising at least two members of the group of the active ingredients naturally occurring in and isolatable from Banisteriopsis caapi, Psychotria viridis and/or Diplopterys cabrerana, consisting of harmine, harmaline, d-tetrahydroharmine, N,N-dimethyltryptamine (DMT), mono-N-methyltryptamine, 5-methoxy-N,N-dimethyltryptamine, 5-hydroxy-N,N-dimethyltryptamine, 2-methyl-1,2,3,4-tetrahydro-β-carboline, harmol, harmalol, tetrahydroharmol, as well as their natural and unnatural stereoisomers and racemates, available in solid, liquid or semi-solid form, characterized in that at least one of the active ingredients is selected from the group of β-carbolines consisting of harmine, harminole and tetrahydrohamine and its stereoisomers and racemate, and at least one of the active substances is selected from the group containing terminal N-substituted tryptamines consisting of N,N-dimethyltryptamine (DMT), mono-N-methyltryptamine, 5-methoxy-N,N-dimethyltryptamine, 5-hydroxy-N,N-dimethyltryptamine. The active ingredients can be contained independently, in whole or in part-individually and in admixture, together and in several dosage forms, in the form of bases or their natural and synthetic salts, where applicable, or as N-oxides, bound to ion exchangers or another matrix, can be present as complexes and inclusion compounds, can be synthesized and/or can be obtained from any natural plant material by extraction and—the sum of the concentrations of the active ingredients is at least 0.0001%. It was hypothesized that peroral pharmahuasca would be more tolerable compared to traditional ayahuasca, due to the elimination of plant admixtures with unknown toxicity, which are known to cause undesired side effects (e.g. vomiting, nausea, diarrhea). According to Wikipedia (https://en.wikipedia.org/wiki/Pharmahuasca), 50 mg DMT and 100 mg harmaline is usually the recommended dosage per person for pharmahuasca. However, combinations of 50 mg harmaline, 50 mg harmine, and 50 mg DMT have been tested with success. The constituents are put into separate gelatin capsules. The capsule with harmaline/harmine is swallowed first and the capsule containing the DMT is taken 15 to 20 minutes later.

To date, the limiting factor for pharmaceutical applications of pharmahuasca is inability to obtain highly bioavailable formulations or salts of harmine that would be suitable for administration to the patient in need thereof. Among others, the poor and highly heterogenous gastro-intestinal absorption of harmine is caused by its poor solubility in water and its ability to readily crystallize to completely insoluble and thus unabsorbable needles under various gastro-intestinal conditions (e.g. sudden increase in pH, when harmine transits from the acidic stomach into more basic environments (duodenum, ileum, etc.). It was found, that circumventing the gastrointestinal route—e.g. by delivering harmine via the buccal/sublingual route—can dramatically improve overall pharmacokinetic performance. As it is known to the person skilled in the art, in order manufacture sublingual, buccal or oromucosal dosage forms (e.g. orodispersible tablet/films, sublingual drops/spray) the compound has to show a high solubility, in order to load sufficient amount of the compound in to one dose unit (sprays/drops:max. 1 ml/dose; ODT:max. 0.5 ml/dose). The same applies to pharmaceutical applications of harmine alone, which to date have been limited by limited solubility of harmine or its salts.

Thus, to date, the limiting factor for pharmaceutical applications of pharmahuasca is inability to obtain highly bioavailable formulations or salts of harmine that would be suitable for administration to the patient in need thereof.

Document WO 2021/259962 discloses certain compositions and kits comprising harmine and DMT for the treatment of psychiatric disorders.

Document US 2021/401786 discloses certain composition for treating burn wounds, the composition comprising carboxylic acids (such as palmitic acid, stearic acid, oleic acid), harmine, fructose and glucose.

Document Marx Sebastien et al (“Design and synthesis of a new soluble natural [beta]-carboline derivative for preclinical study by intravenous injection”, International Journal of Molecular Sciences, DOI 10.3390/ijms20061491) discloses a harmine derivative developed with the purpose to obtain an improved solubility and good biological activity.

### SUMMARY OF THE INVENTION

It was an objective technical problem of the present invention to provide a formulation of harmine characterized by its improved solubility.

The objective technical problem is solved by embodiments disclosed herein and as characterized in the claims.

The present inventors have surprisingly found that compositions comprising harmine (or a pharmaceutically acceptable salt thereof) and (i) an uronic acid or (ii) a carboxylic acid and a monosaccharide are characterized by significantly improved solubility than the state of the art formulations of harmine, including harmine free base or harmine hydrochloride (see e.g. Table 12 and Table 14). Said compositions show better bioavailability and less subject-to-subject variability in comparison to state of the art composition.

The invention will be summarized in the following embodiments.

In a first embodiment, the present invention relates to a composition comprising harmine or a pharmaceutically acceptable salt thereof and (i) an uronic acid; or (ii) a carboxylic acid and a monosaccharide.

In a second embodiment, the present invention relates to a salt of harmine and uronic acid.

In a third embodiment, the present invention relates to a kit of parts comprising: (a) the composition of the present invention or the salt of the present invention and a pharmaceutically acceptable carrier; and (b) DMT or a pharmaceutically acceptable salt thereof and a pharmaceutically acceptable carrier.

In a fourth embodiment, the present invention relates to a pharmaceutical composition comprising (a) the composition of the present invention or the salt of the present invention; and a pharmaceutically acceptable carrier.

In a fifth embodiment, the present invention relates to a pharmaceutical composition comprising (a) the composition of the present invention or the salt of the present invention; and (b) DMT or a pharmaceutically acceptable salt thereof; and a pharmaceutically acceptable carrier.

In a sixth embodiment, the present invention relates to the composition of the present invention, the salt of the present invention, the kit of parts of the present invention, or the pharmaceutical composition of the present invention for use as a medicament.

In a seventh embodiment, the present invention relates to the composition of the present invention, the salt of the present invention, the kit of parts of the present invention or the pharmaceutical composition of the present invention for use in the treatment and/or prevention of a psychiatric, psychosomatic or somatic disorder.

In an eighth embodiment, the present invention relates to DMT hemisuccinate salt.

In a ninth embodiment, the present invention relates to a method for masking the bitterness of a compound, wherein the compound is harmine or a pharmaceutically acceptable salt thereof, or DMT or a pharmaceutically acceptable salt thereof, the method comprising loading a compound having a bitter taste onto a carrier particle wherein a) the carrier particle comprises a loading cavity and wherein the carrier particle comprises a basic salt; and b) wherein the bitterness of the compound is masked by the carrier particle during oral mucosal absorption.

In a tenth embodiment, the present invention relates to a pharmaceutical composition comprising carrier particles, comprising: a) a carrier particle comprising a loading cavity and comprising of a basic salt; and b) a compound having a bitter taste, wherein the compound is harmine or a pharmaceutically acceptable salt thereof, or DMT or a pharmaceutically acceptable salt thereof, wherein the bitterness of the compound is masked by the carrier particle during oral mucosal absorption.

### BRIEF DESCRIPTION OF FIGURES

FIG. 1 shows XRPD of harmine-HCl (upper) and of Harmine HCl dihydrate.

FIG. 2 shows Microscopic image of Harmine-HCl-starting material-scNF0 (ethHACL001) with polarized light.

FIG. 3 shows chip-DSC (lower) & TGA (upper)-thermograms in the temperature range of 25° C. to 600° C. of Harmine-HCl-starting material-scNF0 (ethHACL001) with a heating rate of 10 K/min (TGA) and 20 K/min (chip-DSC).

FIG. 4 shows TGA-thermogram in the temperature range of 25° C. to 600° C. of Harmine-HCl-starting material-scNF0 (ethHACL001) with a heating rate of 10 K/min.

FIG. 5 shows TGA-thermogram in the temperature range of 25° C. to 500° C. of Harmine-HCl-scNF1 (ethHACL001LAG002) with a heating rate of 10 K/min.

FIG. 6 shows dissolved in water composition comprising harmine and glucuronic acid in the molar ratio of 1:1.

FIG. 7 shows dissolved in water composition comprising harmine and galacturonic acid in molar ratio of 1:1.

FIG. 8 shows dissolved in water composition comprising harmine, glucuronic acid and galacturonic acid in molar ratio of 1:1:1.

FIG. 9 shows composition comprising harmine, glucose and acetic acid in a molar ratio of 1:1:1.5.

FIG. 10 shows composition comprising harmine, fructose and malic acid in a molar ratio of 1:0.5:0.5.

FIG. 11 shows SEM image of harmine glucuronate.

FIG. 12 shows SEM picture of A) unsuccessful loading with Harmine HCl, B) successfully loaded Harmine HCl and C) successfully loaded Harmine glucuronate.

FIG. 13 shows blood plasma profiles following oral administration of harmine HCL (A) and sublingual administration of F3 (B), F4 (C) and F6 (D).

FIG. 14 shows a schematic depiction for the choice of solvents

FIG. 15 shows XRPD of Harmine-starting material-scNF0 (ethHAR001).

FIG. 16 shows XRPD of Harmine-succinic acid-scNF1 (ethHAR001EVA018).

FIG. 17 shows XRPD of Harmine-L(+)-tartaric acid-scNF2 (ethHAR001GRI002).

FIG. 18 shows XRPD of Harmine-L-ascorbic acid-scNF3 (ethHAR001GRI003).

FIG. 19 shows XRPD of Harmine-L(−)-malic acid-scNF4 (ethHAR001GRI004).

FIG. 20 shows XRPD of Harmine-citric acid-scNF5 (ethHAR001GRI017).

FIG. 21 shows XRPD of Harmine-L(+)-tartaric acid-scNF6 (ethHAR001EVA014).

FIG. 22 shows XRPD of Harmine-L(−)-malic acid-scNF7+scNF4 (ethHAR001EVA016).

FIG. 23 shows XRPD of Harmine-methanesulfonic acid-scNF8 (ethHAR001EXP113).

FIG. 24 shows XRPD of Harmine-sulfuric acid-scNF9 (ethHAR001EXP114).

FIG. 25 shows XRPD of Harmine-L(+)-tartaric acid scNF10 (ethHAR001GRI014).

FIG. 26 shows XRPD of Harmine-L-ascorbic acid-scNF11 (ethHAR001GRI015).

FIG. 27 shows XRPD of Harmine-phosphoric acid-ScNF12 (ethHAR001EXP115).

FIG. 28 shows XRPD of Harmine-L(+)-tartaric acid-scNF13 (ethHAR001EVA019).

FIG. 29 shows XRPD of Harmine-L-ascorbic acid-scNF14 (ethHAR001EVA020).

FIG. 30 shows microscopic image of Harmine-succinic acid-scNF1 (ethHAR001GRI001) with polarized light.

FIG. 31 shows microscopic image of Harmine-L(+)-tartaric acid-scNF2 (ethHAR001GRI002) with polarized light.

FIG. 32 shows microscopic image of Harmine-L-ascorbic acid-scNF3 (ethHAR001GRI003) with polarized light.

FIG. 33 shows Microscopic image of Harmine-L(−)-malic acid-scNF4 (ethHAR001GRI004) with polarized light.

FIG. 34 shows Microscopic image of Harmine-citric acid-scNF5 (ethHAR001GRI005) with polarized light.

FIG. 35 shows DSC and TGA-thermograms in the temperature range of 25° C. to 300° C. of Harmine-starting material-scNF0 (ethHAR001) with heating and cooling rate of 10 K/min.

FIG. 36 shows DSC-thermogram in the temperature range of 25° C. to 160° C. of Harmine-succinic acid-scNF1 (ethHAR001EVA018) with heating and cooling rate of 10 K/min.

FIG. 37 shows DSC and TGA-thermograms in the temperature range of 25° C. to 350° C. of Harmine-L(+)-tartaric acid-scNF2 (ethHAR001GRI002) with heating and cooling rate of 10 K/min.

FIG. 38 shows DSC and TGA-thermograms in the temperature range of 25° C. to 350° C. of Harmine-L-ascorbic acid-scNF3 (ethHAR001GRI003) with heating and cooling rate of 10 K/min.

FIG. 39 shows DSC and TGA-thermograms in the temperature range of 25° C. to 350° C. of Harmine-L(−)-malic acid-scNF4 (ethHAR001GRI004) with heating and cooling rate of 10 K/min.

FIG. 40 shows DSC and TGA-thermograms in the temperature range of 25° C. to 350° C. of Harmine-citric acid-scNF5 (ethHAR001GRI017) with heating and cooling rate of 10 K/min.

FIG. 41 shows DSC and TGA-thermograms in the temperature range of 25° C. to 350° C. of Harmine-L(+)-tartaric acid-scNF6 (ethHAR001EVA014) with heating and cooling rate of 10 K/min.

FIG. 42 shows DSC and TGA-thermograms in the temperature range of 25° C. to 350° C. of Harmine-L(−)-malic acid-scNF7+scNF4 (ethHAR001EVA016) with heating and cooling rate of 10 K/min.

FIG. 43 shows DSC and TGA-thermograms in the temperature range of 25° C. to 150° C. of Harmine-methanesulfonic acid-scNF8 (ethHAR001EXP113) with heating and cooling rate of 10 K/min.

FIG. 44 shows DSC and TGA-thermograms in the temperature range of 25° C. to 350° C. of Harmine-sulfuric acid-scNF9 (ethHAR001EXP114) with heating and cooling rate of 10 K/min.

FIG. 45 shows DSC and TGA-thermograms in the temperature range of 25° C. to 350° C. of Harmine-L-tartaric acid-scNF10 (ethHAR001GRI014) with heating and cooling rate of 10 K/min.

FIG. 46 shows DSC-thermogram in the temperature range of 25° C. to 150° C. of Harmine-L-ascorbic acid-scNF11 (ethHAR001GRI015) with heating and cooling rate of 10 K/min.

FIG. 47 shows DSC and TGA-thermograms in the temperature range of 25° C. to 350° C. of Harmine-phosphoric acid-scNF12 (ethHAR001EXP115) with heating and cooling rate of 10 K/min.

FIG. 48 shows IR-spectrum of Harmine-starting material-scNF0 (ethHAR001).

FIG. 49 shows IR-spectrum of Harmine-succinic acid-ScNF1 (ethHAR001GRI001).

FIG. 50 shows IR-spectrum of Harmine-L(+)-tartaric acid-scNF2 (ethHAR001GRI002).

FIG. 51 shows IR-spectrum of Harmine-L-ascorbic acid-ScNF3 (ethHAR001GRI003).

FIG. 52 shows IR-spectrum of Harmine-L(−)-malic acid-scNF4 (ethHAR001GRI004).

FIG. 53 shows IR-spectrum of Harmine-citric acid-scNF5 (ethHAR001GRI005).

FIG. 54 shows IR-spectrum of Harmine-phosphoric acid-scNF12 (ethHAR001EXP115).

FIG. 55 shows PXRD measurement of the experiments used for solubility determination. Harmine glucuronate (ethHARGCS002, orange), harmine phosphate (ethHAR001EXP115R4, blue) and harmine hydrochloride (ethHACL001, green).

FIG. 56 shows the 1H NMR of the harmine glucuronate (ethHARGCS002) after its preparation. Glucuronic acid and harmine are present in a molar ratio of 1:1.

FIG. 57 shows the 1H NMR of the harmine glucuronate (ethHARGCS002H2OSOL002T25) after the solubility experiment was performed. Glucuronic acid and harmine are still present in a 1:1 molar ratio. The broad signal between 2.75 and 4.00 ppm is caused by a slightly increased water content after the experiment.

FIG. 58 shows the PXRD of the harmine glucuronate (ethHARGCS002H2OSOL002T25, black) after the solubility experiment performed compared to the PXRD of the pure harmine (ethHAR001, red). The phase analysis shows that no signals of the pure harmine can be recovered. This could indicate salt or cocrystal formation.

FIG. 59 shows solubility of different harmine salts at 25° C. and 37° C. in EtOH

FIG. 60 shows solubility of different harmine salts at 25° C. and 37° C. in EtOH

FIG. 61 shows IR-Spectra comparison of the sample ethHAR001EXP115-scNF12 (phosphoric acid salt), potassium-hydrogen phosphate and potassium-dihydrogen phosphate.

FIG. 62 shows 1H-NMR-spectrum of DMT hemisuccinate Form B in d-DMSO.

FIG. 63 shows XRPD of unloaded TIP particles compared with the XRD patterns of DMT hemisuccinate crystal Form A and TIP particles loaded with DMT hemisuccinate (4 hours additional drying at 50° C.). Several additional signals are present which can be assigned to DMT hemisuccinate Form A.

FIG. 64 shows PK profiles of blood plasma harmine concentration for two representative subjects receiving a high dose of sublingual DMT/harmine glucuronate.

FIG. 65 shows Harmine HCL loaded TIP particles (upper) and Harmine glucuronate loaded TIP particles (lower)

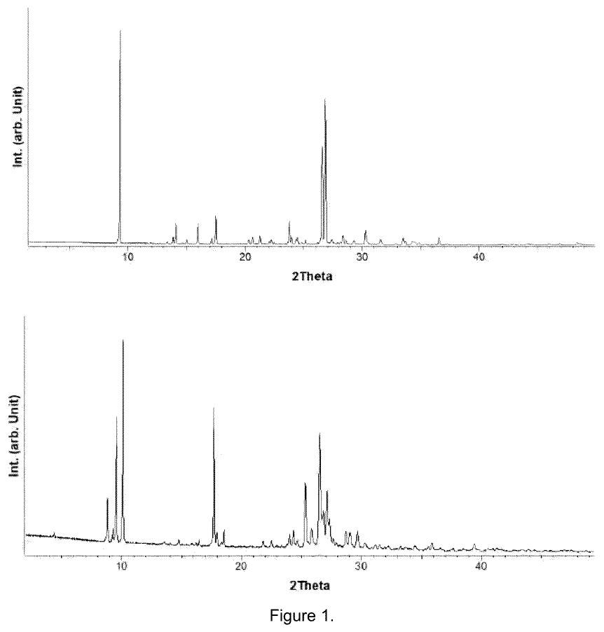

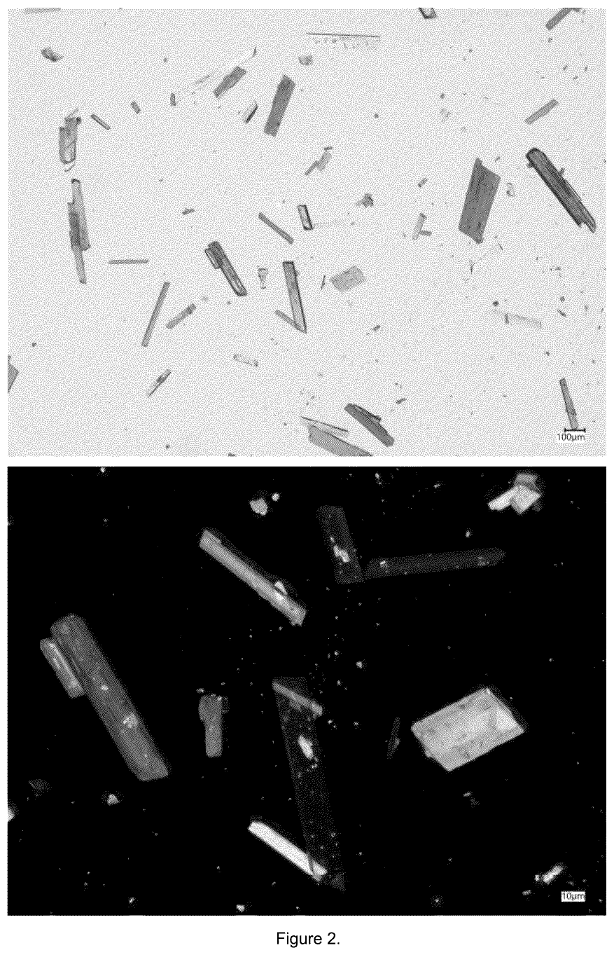

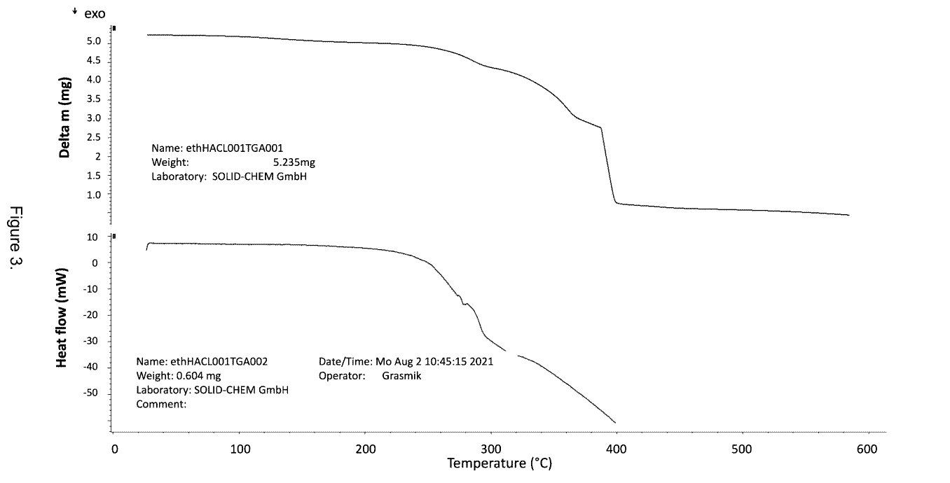

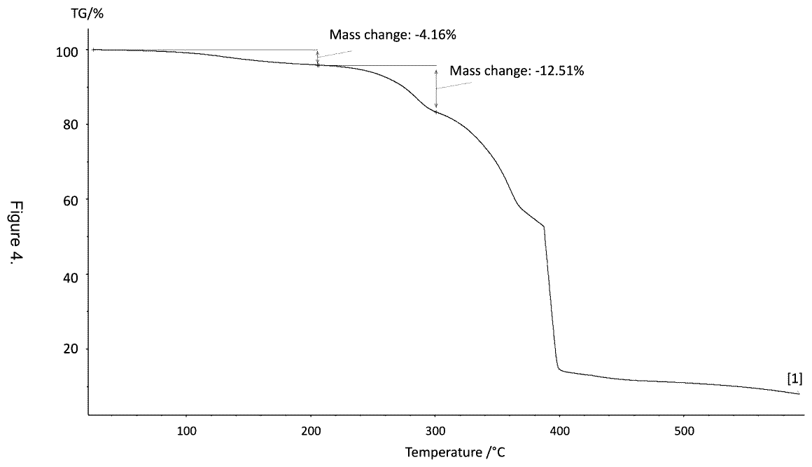

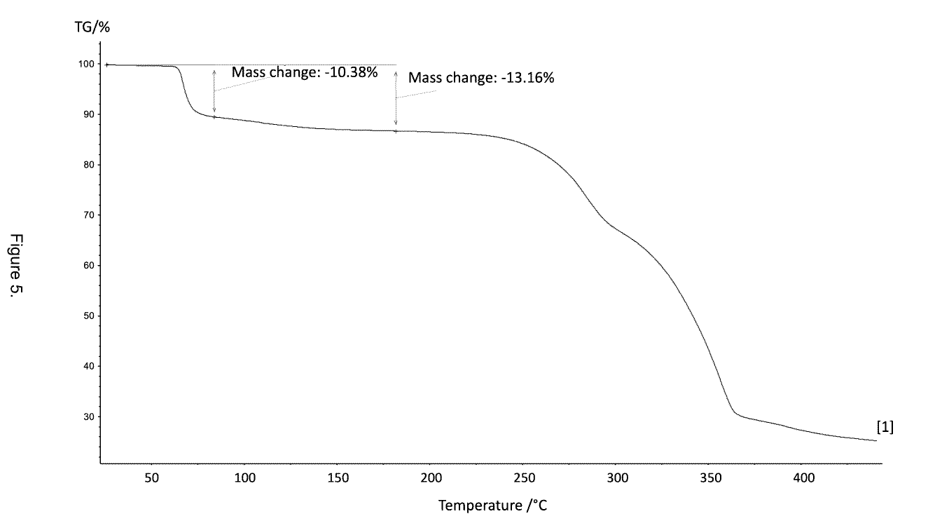

### DETAILED DESCRIPTION OF THE INVENTION

The invention will be described in detail in the following. It is to be understood that all the features recited hereinbelow may be combined, unless explicitly indicated to the contrary.

In one embodiment, the present invention relates to a composition comprising harmine or a pharmaceutically acceptable salt thereof and (i) an uronic acid; or (ii) a carboxylic acid and a monosaccharide.

Harmine is a compound of the formula:

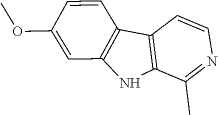 [SMILES: COc1ccc2c(c1)[nH]c1c(C)nccc12]

The compound as shown in the formula hereinabove may also be referred to as harmine free base, or harmine FB. Harmine (7-methoxy-1-methyl-9H-pyrido[3, 4-b]-indole), also known as banisterine or as telepathine, is an alkaloid that occurs in a number of different plants, including harmel (Peganum harmala) or Banisteriopsis caapi. It belongs to a group of beta-carbolines. Harmine reversibly inhibits monoamine oxidase A (MAO-A), but it does not inhibit the monoamine oxidase B (MAO-B).

In the compositions, the pharmaceutical compositions, the kits of parts and the methods of the present invention harmine is primarily used as a selective reversible inhibitor of MAO-A. Other nervous system effects include increased Brain-derived neurotrophic factor (BDNF) protein levels as well as analgesic and antinociceptive effects. Ayahuasca constituents were further shown to stimulate neuronal cell proliferation and to prevent neuronal damage and improve cell viability. Besides those neuroprotective effects, harmine and other beta-carbolines might be able to raise dopamine levels in the CNS and thus be effective to alleviate the symptoms of parkinsonism. Other pharmacological activities of harmine include anti-inflammatory, antidiabetic, and antitumor activities. Specifically, antimicrobial (antiprotozoal, antibacterial, insecticidal, and antifungal) activity has been documented for P. harmala-derived beta-carbolines. Various other studies have shown antineoplastic, antiproliferative, antioxidant, as well as immune-modulatory (anti-inflammatory) effects for harmala alkaloids. In addition, cardiovascular effects were reported such as vasorelaxant, antihypertensive, and negative inotropic effects, as well as anti-angiogenic inhibitory and anti-platelet aggregation effects (Moloudizargari M, Mikaili P, Aghajanshakeri S, Asghari M H, Shayegh J. Pharmacological and therapeutic effects of Peganum harmala and its main alkaloids. Pharmacogn Rev. 2013 July; 7(14):199-212. doi: 10.4103/0973-7847.120524. PMID: 24347928; PMCID: PMC3841998; Zhang, L., Li, D. & Yu, S. Pharmacological effects of harmine and its derivatives: a review. Arch. Pharm. Res. 43, 1259-1275 (2020). https://doi.org/10.1007/s12272-020-01283-6).

Several structural analogues of harmine include harmaline, tetrohydroharmine, harmol, harmalol, tetrahydroharmol, 2-methyl-1,2,3,4-tetra-hydro-β-carboline. It is noted that the analogues of harmine listed herein are all MAO-A inhibitors. Therefore, it is further envisaged that harmaline, tetrohydroharmine, harmol, haramolol, tetrahydroharmol, 2-methyl-1,2,3,4.tetra-hydro-β-carboline may also be used in the compositions, the pharmaceutical compositions, the kits of parts and/or the methods of the present invention, replacing harmine. It is accordingly expected that harmaline, tetrohydroharmine, harmol, haramolol, tetrahydroharmol, 2-methyl-1,2,3,4.tetra-hydro-β-carboline would also benefit from the approaches described herein to increase solubility and/or bioavailability of their formulations.

Pharmaceutically acceptable salt of the compounds discussed herein (in particular of harmine or DMT), may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of harmine include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of harmine is a hydrochloride salt.

Preferably, the harmine or a pharmaceutically acceptable salt thereof is a harmine free base. Thus accordingly, preferably the present invention relates to a composition comprising harmine and (i) an uronic acid; or (ii) a carboxylic acid and a monosaccharide.

Compounds referred to herein or pharmaceutically acceptable salts thereof may exist as hydrates, or solvates thereof. Accordingly, solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

It is however to be understood that in one embodiment of the invention, the composition may comprise a pharmaceutically acceptable salt of harmine, for example harmine hydrochloride.

The composition of the present invention may comprise (i) an uronic acid. Preferably, uronic acid (which also may be referred to as alduronic acid) is herein understood as a sugar acid comprising both carbonyl group (i.e. a —CHO group or a —CO— group, preferably when present in its linear form) and a carboxylic acid functional group (i.e., —COOH group). One exemplary uronic acid is glucuronic acid, obtainable from glucose upon the oxidation of its terminal hydroxyl group. Glucuronic acid can be presented using the following Fischer projection:

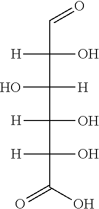 [SMILES: O=CC(O)C(O)C(O)C(O)C(=O)O]

As it is apparent to the skilled person, such a sugar may further have a cyclic form, for example:

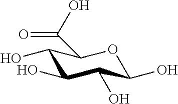

Uronic acids derived from hexoses (i.e., monosaccharides characterized by the presence of six carbon atoms) may be referred to as hexuronic acids. Uronic acids derived from pentoses (i.e., monosaccharides characterized by the presence of five carbon atoms) may be referred to as penturonic acids. Monosaccharide is preferably as defined hereinbelow.

Accordingly and preferably, in the composition of the present invention, the uronic acid is a penturonic acid or a hexuronic acid. More preferably, in the composition of the present invention, the uronic acid is a hexuronic acid. Even more preferably, in the composition of the present invention, the uronic acid is glucuronic acid or galacturonic acid. Still more preferably, the uronic acid is glucuronic acid.

Further disclosed are the compositions of the present invention wherein the uronic acid is replaced with aldonic acid or aldaric acid.

Further encompassed by the present invention are embodiments wherein the uronic acid, as described hereinabove, is present as a pharmaceutically acceptable salt, as described hereinabove. However, preferably the uronic acid is present as free acid.

Preferably, the composition of the present invention comprises (i) an uronic acid.

Preferably, the composition of the present invention comprises harmine and an uronic acid, preferably wherein the composition comprises a salt of harmine and uronic acid. The uronic is as defined hereinabove.

The composition of the present invention may comprise (ii) a carboxylic acid and a monosaccharide. Preferably, said carboxylic acid and said monosaccharide are preferably present in a molar ratio of between 0.5 and 2.0, more preferably present in a molar ratio of about 1:1.

Carboxylic acid is preferably a compound of formula R—COOH, wherein R is selected from C₁₋₆ alkyl, C₂₋₆ alkenyl, C₂₋₆ alkynyl, and C₃₋₆ cycloalkyl, wherein said alkyl, said alkenyl, said alkynyl and said cycloalkyl are each optionally substituted with one or more optional substituents selected from Hal, —OH, —CN, —O—(C₁₋₆ alkyl), —SH, —S(C₁₋₆ alkyl), —NH₂, —NH(C₁₋₆ alkyl), —N(C₁₋₆ alkyl)(C₁₋₆ alkyl), —CO(C₁₋₆ alkyl), —COOH, —COO(C₁₋₆ alkyl), —CONH₂, —CONH(C₁₋₆ alkyl), and —CON(C₁₋₆ alkyl)(C₁₋₆ alkyl), preferably selected from —OH, —CN, —SH, —NH₂, —COOH, and —CONH₂, more preferably selected from —OH, —NH₂, —COOH, and —CONH₂, even more preferably selected from —OH, and —COOH. It is further preferred that not more than one optional substituent is selected from —CO(C₁₋₆ alkyl), —COOH, —COO(C₁₋₆ alkyl), —CONH₂, —CONH(C₁₋₆ alkyl), and —CON(C₁₋₆ alkyl)(C₁₋₆ alkyl).

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members (e.g., cyclopropyl or cyclohexyl).

As used herein, the term “Hal” or “halogen” refers to fluoro (—F), chloro (—Cl), bromo (—Br), or iodo (—I).

Preferably, R is selected from C₁₋₆ alkyl and C₂₋₆ alkenyl, wherein said alkyl or said alkenyl are each optionally substituted with one or more optional substituents selected from Hal, —OH, —CN, —O—(C₁₋₆ alkyl), —SH, —S(C₁₋₆ alkyl), —NH₂, —NH(C₁₋₆ alkyl), —N(C₁₋₆ alkyl)(C₁₋₆ alkyl), —CO(C₁₋₆ alkyl), —COOH, —COO(C₁₋₆ alkyl), —CONH₂, —CONH(C₁₋₆ alkyl), and —CON(C₁₋₆ alkyl)(C₁₋₆ alkyl), preferably selected from —OH, —CN, —SH, —NH₂, —COOH, and —CONH₂, more preferably selected from —OH, —NH₂, —COOH, and —CONH₂, even more preferably selected from —OH, and —COOH. It is further preferred that not more than one optional substituent is selected from —CO(C₁₋₆ alkyl), —COOH, —COO(C₁₋₆ alkyl), —CONH₂, —CONH(C₁₋₆ alkyl), and —CON(C₁₋₆ alkyl)(C₁₋₆ alkyl).

Preferably the carboxylic acid is selected from formic acid, acetic acid, propionic acid, butyric acid, valeric acid, caproic acid, enanthic acid, glycolic acid, lactic acid, citric acid, 2-hydroxypropionic acid, 3-hydroxypropionic acid, 3-hydroxybutyric acid, oxalic acid, malonic acid, succinic acid, glutaric acid, adipic acid, pimelic acid, suberic acid, tartaric acid, malic acid, maleic acid, fumaric acid, and glutatonic acid.

More preferably, the carboxylic acid is selected from acetic acid, propionic acid, butyric acid, lactic acid, oxalic acid, malonic acid, succinic acid, glutaric acid, malic acid, maleic acid and fumaric acid.

Even more preferably, the carboxylic acid is selected from acetic acid, propionic acid, lactic acid, and malic acid.

Even more preferably, the carboxylic acid is malic acid or acetic acid.

In one embodiment, the carboxylic acid is an amino acid, accordingly a compound of formula R—COOH, wherein R is C₁₋₆ alkyl, substituted with —NH₂, at a carbon atom adjacent to the COOH group of said R—COOH, further optionally substituted with one optional substituent selected from —OH, —NH₂, —COOH, and —CONH₂. Preferably, the carboxylic acid is an L-natural amino acid, as known to the skilled person.

Accordingly, if the carboxylic acid is an amino acid, the carboxylic acid is preferably selected from glutamate, and aspartate.

Further encompassed by the present invention is an embodiment wherein said carboxylic acid as referred to hereinabove is present as a pharmaceutically acceptable salt. However, preferably the carboxylic acid is present as a free acid.

Monosaccharide is preferably defined as a simple sugar, that is a compound of a linear and unbranched carbon skeleton with one carbonyl functional group and one hydroxyl functional group on each of the remaining carbon atoms. For certain carbon atoms, hydroxy group may be absent. Accordingly, a monosaccharide is a compound of formula

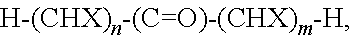

wherein n+m+1 is preferably selected from 3, 4, 5, 6 and 7, wherein each X is independently H or —OH, provided that at least two instances of X are OH and not more than 2 instances of X are H, preferably wherein not more than 1 instance of X is H, more preferably wherein each X is —OH.

Preferably, the monosaccharide is a hexose or a pentose. A hexose is a monosaccharide as defined hereinabove wherein n+m+1=6, and preferably wherein each instance of X is —OH. A pentose is a monosaccharide as defined hereinabove, wherein n+m+1=5 and preferably wherein each instance of X is —OH).

More preferably, the monosaccharide is a hexose.

Even more preferably, the monosaccharide is glucose or fructose.

Accordingly and preferably, in the composition of the present invention, the carboxylic acid is malic acid or acetic acid, and/or the monosaccharide is glucose or fructose.

In one embodiment of the present invention, the composition of the present invention comprises (ii) a carboxylic acid and a monosaccharide. Preferably, said carboxylic acid and said monosaccharide are preferably present in a molar ratio of between 0.5 and 2.0, more preferably present in a molar ration of about 1:1.

Preferably, in the composition of the present invention, harmine and the uronic acid in (i), or harmine and the carboxylic acid in (ii), are present in a molar ratio of between 0.5 and 2.0, preferably in a molar ratio of about 1:1. Accordingly, if the composition of the present invention comprises harmine and (i) a uronic acid, said harmine and the uronic acid are present in a molar ratio of between 0.5 and 2.0, preferably in a molar ratio of about 1:1. Further accordingly, if the composition of the present invention comprises harmine and (ii) a carboxylic acid and a monosaccharide, said harmine and the carboxylic acid in (ii), are present in a molar ratio of between 0.5 and 2.0, preferably in a molar ratio of about 1:1.

Preferably, the composition of the present invention is an amorphous composition. Accordingly, the amorphous composition as referred to herein is a composition that has no detectable crystal structure. It has been postulated by the present inventors that due to inability of the components of the composition, e.g. harmine and an uronic acid, or harmine and a carboxylic acid and a monosaccharide, to form a detectable crystal structure, the solubility of the composition and accordingly the solubility of harmine, is vastly improved in comparisons with the compositions of the prior art.

The composition of the present invention, as defined hereinabove, may comprise a natural deep eutectic solvent or a co-amorphous system. The use of natural deep eutectic solvents for improving bioavailability of therapeutic compounds (Molecules, 2016 Nov. 14; 21(11):1531. doi: 10.3390/molecules21111531) as well as co-amorphous systems with improved solubility (Crystal Growth & Design 2021 21 (6), 3280-3289, DOI: 10.1021/acs.cgd.1c00015) have been recently reported.

In one embodiment, the present invention relates to a salt of harmine and uronic acid. It is to be understood that the salt of harmine and uronic acid is preferably characterized by 1:1 stoichiometry. Preferred salts of harmine and uronic acid are harmine glucuronate and harmine galacturonate.

In a further embodiment, the present invention relates to a pharmaceutical composition comprising (a) the composition of the present invention or the salt of the present invention; and a pharmaceutically acceptable carrier. Herein, the reference is made to the composition or the salt comprising harmine.

In a further embodiment, the present invention relates to a pharmaceutical composition comprising (a) the composition of the present invention or the salt of the present invention (comprising harmine); (b) DMT or a pharmaceutically acceptable salt thereof; and a pharmaceutically acceptable carrier.

As referred to herein, DMT is a compound of formula:

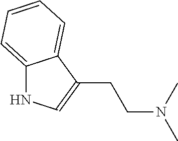

Accordingly, DMT (N,N-dimethyltryptamine) is a psychedelic substance that is a structural analogue of serotonin and melatonin. DMT is also a structural and functional analogue of other psychedelic substances, including bufotenin (5-hydroxy-N,N-dimethyltryptamine), psilocybin (phosphate ester of 4-hydroxy-N,N-dimethyltryptamine) and psilocin (4-hydroxy-N,N-dimethyltryptamine). Further known analogues of DMT include mono-N-methyltryptamine. The analogues of DMT listed herein also show activity as psychedelic agents. Furthermore, the analogues of DMT listed herein are all mono-amines, and as such are potential substrates of MAO-A monoamine oxidase. Therefore, it is further envisaged that psilocybin, psilocin, and mono-N-methyltryptamine may also be used in the compositions, the pharmaceutical compositions, the kits of parts and/or the methods of the present invention, replacing DMT. In particular, it is envisaged that psilocin or psilocybin may be used in the compositions, the pharmaceutical compositions, the kits of parts and/or the methods of the present invention, replacing DMT. It is conceivable to the skilled person, that such pharmaceutical composition, composition or kit of parts comprising psilocin or psilocybin instead of DMT could be used to treat the diseases that can be treated with the pharmaceutical compositions, compositions or the kits of parts of the present invention.

As referred to herein, a pharmaceutically acceptable salt of DMT is as defined hereinabove. Particularly preferred pharmaceutically acceptable salt of DMT is DMT hemifumarate or DMT hemisuccinate. Still more preferably, the pharmaceutically acceptable salt of DMT is DMT hemisuccinate.

Accordingly, the present invention relates to a salt of DMT, wherein said salt is DMT hemifumarate or DMT hemisuccinate. It is particularly preferred that the salt of DMT is DMT hemisuccinate. The present inventors have obtained DMT hemisuccinate in amounts sufficient for physicochemical characterization. The present inventors have further shown that crystallization of a specific crystal form of DMT hemisuccinate can occur in a carrier particle, preferably in a template inverted particle.

As understood herein, whenever a reference to DMT or a pharmaceutically acceptable salt thereof is made, it is also to be understood as a narrower reference to DMT hemifumarate or DMT hemisuccinate, or more preferably DMT hemisuccinate. As understood herein and as apparent to the skilled person, in DMT hemisuccinate salt, two molecules of DMT per each molecule of succinate are present. In other words, stoichiometry of DMT to succinate (i.e. molar ratio of DMT to succinate) is 2:1. Similarly, as understood herein and as apparent to the skilled person, in DMY hemifumarate salt two molecules of DMT per each molecule of fumarate are present. In other words, stoichiometry of DMT to fumarate (i.e. molar ratio of DMT to fumarate) is 2:1.

The present invention further provides DMT hemisuccinate in a crystal form A and in a crystal form B. These crystal forms are as characterized hereinbelow.

A polymorphic form of DMT hemisuccinate having been referred to as crystal form A is characterized by the X-ray powder diffraction pattern (Cu-Kα₁) comprising a peak at about 16.14±0.2°. Preferably, the X-ray powder diffraction pattern (Cu-Kα₁) further comprises one or more peaks selected from 13.50±0.2°, 17.84±0.2°, 19.67±0.2°, 21.81±0.2°, 23.19±0.2°, and 25.36±0.2°. More preferably, said X-ray powder diffraction pattern preferably comprises at least 3, at least 4, at least 5, or all of the listed peaks. Even more, the X-ray diffraction pattern (Cu-Kα₁) further comprises one or more peaks selected from 11.66±0.2°, 12.31±0.2°, 18.77±0.2°, 19.38±0.2°, 20.25±0.2°, 21.81±0.2°, 22.45±0.2°, 23.57±0.2°, 24.38±0.2°, 25.79±0.2°, 28.02±0.2°, 29.79±0.2°, and 30.82±0.2°. Still more preferably, said X-ray powder diffraction pattern preferably comprises at least 3, at least 4, at least 5, or all of the listed peaks. Further peaks are as provided in the experimental section.

A polymorphic form of DMT hemisuccinate having been referred to as crystal form B is characterized by the X-ray powder diffraction pattern (Cu-Kα₁) comprising a peak at about 15.57±0.2°. Preferably, the X-ray powder diffraction pattern (Cu-Kα₁) further comprises one or more peaks selected from 10.09±0.2°, 16.52±0.2°, 16.82±0.2°, 17.06±0.2°, 19.34±0.2°, 19.93±0.2°, 21.13±0.2°, 22.91±0.2°, and 23.45±0.2°. More preferably, said X-ray powder diffraction pattern preferably comprises at least 3, at least 4, at least 5, or all of the listed peaks. Even more, the X-ray diffraction pattern (Cu-Kα₁) further comprises one or more peaks selected from 21.38±0.2°, 22.07±0.2°, 23.81±0.2°, 24.14±0.2°, 28.60±0.2°, and 28.74±0.2°. Still more preferably, said X-ray powder diffraction pattern preferably comprises at least 3, at least 4, at least 5, or all of the listed peaks. Further peaks are as provided in the experimental section. It is to be understood that all diffraction angles provided when discussing the X-ray powder diffraction patterns are given as 2θ.

The hemisuccinate salt of DMT, in particular its crystal forms A and B, are particularly advantageous for use in pharmaceutical applications due to good aqueous solubility of said salt. These crystal forms can be obtained as described in the examples section.

According to the present invention, in the pharmaceutical composition comprising (a) and (b), (a) and (b) will be mixed together or packaged together, being suitable for being administered together. It is known to the skilled person that small molecule drugs can be administered through peroral route of administration, parenteral route of administration (including intravenous route of administration, intramuscular route of administration, and subcutaneous route of administration), nasal (or intranasal) route of administration, ocular route of administration, transmucosal route of administration (buccal route of administration, sublingual route of administration, vaginal route of administration, and rectal route of administration), inhalation route of administration and transdermal route of administration. Herein, if (a) and (b) are comprised within one composition, they are typically formulated for the same route of administration.

In a further embodiment, the present invention relates to a kit of parts comprising: (a) the composition of the present invention or the salt of the present invention and a pharmaceutically acceptable carrier; and (b) DMT or a pharmaceutically acceptable salt thereof and a pharmaceutically acceptable carrier.

In the present invention, the kit of parts refers to a combination of individual components (a) and (b) which are kept physically separate but adjacent. The skilled person will understand that the components (parts) of the kit may be combined before administration, that the components (parts) may be administered simultaneously, or that the components (parts) of the kit may be administered sequentially. In the case of sequential administration, the components (parts) of the kit are typically to be administered preferably within a time range of between 15 minutes and 120 minutes in order to achieve the effects of the present invention. The components of the kit of parts can be formulated for different routes of administration. It is known to the skilled person that small molecule drugs can be administered through peroral route of administration, parenteral route of administration (including intravenous route of administration, intramuscular route of administration, and subcutaneous route of administration), nasal (or intranasal) route of administration, ocular route of administration, transmucosal route of administration (buccal route of administration, sublingual route of administration, vaginal route of administration, and rectal route of administration), transdermal route of administration, inhalation route of administration and transdermal route of administration. It should be noted that herein, oral route of administration may refer to peroral route of administration, buccal route of administration and/or sublingual route of administration. Herein, components (a) and (b) may be formulated for administration through any of these routes of administration. It will be understood that (a) and (b) can be formulated for administration using the same route of administration, it will be further understood that (a) and (b) can be formulated for administration using different routes of administration.

The dosage will depend on the route of administration, the severity of the disease, age and weight of the subject and other factors normally considered by the attending physician, when determining the individual regimen and dosage level for a particular patient or subject. The parts of the kit of parts or the pharmaceutical composition of the present invention may be administered via any route, including parenteral, intramuscular, subcutaneous, topical, transdermal, intranasal, intravenous, sublingual or intrarectal administration.

Preferably, with the present invention, harmine and/or DMT are to be administered sublingually or buccally, more preferably harmine and/or DMT are to be administered sublingually. Further preferably, when reference is made to harmine and/or DMT, harmine and DMT is preferably meant.

The parts of the kit of parts of the invention or the pharmaceutical composition of the invention may be prepared by mixing suitably selected and pharmaceutically acceptable excipients, vehicles, adjuvants, additives, surfactants, desiccants or diluents known to those well-skilled in the art, and can be suitably adapted for peroral, transmucosal, parenteral or topical administration. Typically and preferably the parts of the kit or the pharmaceutical composition of the invention are administered in the form of a tablet, orodispersible tablet, mucoadhesive film, lyophilizates, capsule, sachets, powder, granule, pellet, peroral or parenteral solution, suspension, suppository, ointment, cream, lotion, gel, paste and/or may contain liposomes, micelles and/or microspheres.

The term “pharmaceutically acceptable” indicates that the compound or composition, typically and preferably the salt or carrier, must be compatible chemically or toxicologically with the other ingredient(s), typically and preferably with the inventive composition or with the parts of the inventive kit of parts, when typically and preferably used in a formulation or when typically and preferably used for treating the animal, preferably the human, therewith. Preferably, the term “pharmaceutically acceptable” indicates that the compound or composition, typically and preferably the salt or carrier, must be compatible chemically and toxicologically with the other ingredient(s), typically and preferably with the inventive composition or with the parts of the inventive kit of parts, when typically and preferably used in a formulation or when typically and preferably used for treating the animal, preferably the human, therewith. It is noted that pharmaceutical compositions can be formulated by techniques known to the person skilled in the art, such as the techniques published in “Remington: The Science and Practice of Pharmacy”, Pharmaceutical Press, 22^{nd} edition.

The pharmaceutically acceptable carrier of the parts (a) and (b) of the kit of parts of the present invention or of the pharmaceutical composition of the present invention is without limitation any pharmaceutically acceptable excipient, vehicle, adjuvant, additive, surfactant, desiccant or diluent. Suitable pharmaceutically acceptable carriers are magnesium carbonate, magnesium stearate, talc, lactose, sugar, pectin, dextrin, starch, tragacanth, methyl cellulose, hydroxy-propyl-methyl-cellulose, sodium carboxymethyl cellulose, a low-melting wax, cocoa butter. Pharmaceutically acceptable carriers of the invention can be solid, semi-solid or liquid.

According to the present invention, the compositions, the salts and parts of the kits of parts of the present invention may be formulated by using carrier particles. The carrier particles are not to be particularly limited and any carrier particles known to the skilled person can be used within the invention.

The term “carrier particle”, as used herein, refers to a material that is nontoxic or not substantially toxic to a subject, which can be used to improve a desired drug delivery property of a solid pharmaceutical composition. The carrier particle described herein has no or no substantial therapeutic effect upon administration to a subject unless it is loaded with a therapeutic agent. In some embodiments, the carrier particle described herein is pharmacologically inert unless it is loaded with a therapeutic agent. In some embodiments, the carrier particle described herein does not or not substantially dissolve in water. The desired drug delivery properties described herein of the solid pharmaceutical composition include, without limitation, effectiveness, safety, pharmacokinetic properties (e.g., bioavailability), physical stability, chemical stability, drug loading capacity, and/or disintegration time. In some embodiments, the desired drug delivery properties of a solid pharmaceutical composition are physical stability, drug loading capacity, and disintegration time. In some embodiments, the desired drug delivery properties of a solid pharmaceutical composition are high drug loading capacity of the solid pharmaceutical composition (e.g., the drug loading capacity of v/v≥50%, ≥55%, ≥60%, ≥65%, ≥70%, ≥75%, ≥80%, preferably ≥60%, more preferably between 60%, and 85%), low disintegration time of the solid pharmaceutical composition (e.g., ≤15 s, ≤14 s, ≤13 s, ≤12 s, ≤11 s, ≤10 s, preferably ≤10 s) and/or physical stability (e.g., tablet hardness of ≥200N, ≥210N≥220N, ≥230N, ≥240N, or ≥250N, for an 11 mm tablet or ≥40N, ≥50N, ≥60N for a 6 mm tablet, preferably ≥50N for an 6 mm tablet. A carrier particle according as described herein, can have any shape, preferably a carrier particle as described herein has a shape similar to that of a sphere, a spheroid, and/or a bead. Removal of the template material can result in at least one pore in the otherwise largely uniform structure. The carrier particle preferably can form a hollow structure in a dry environment. As such, the carrier particle described herein does not or not substantially collapse upon drying.

It is to be understood that the compositions, the salts and parts of the kits of parts of the present invention may be that is formulated as carrier particles may be formulated as orodispersible tablet. Accordingly, said carrier particles loaded with said composition, said salt or said part(s) of the kits of parts of the present invention may be may be compacted together to form a tablet. Depending on the disintegration properties of the tablet, said tablet may be orodispersible. The skilled person is capable of formulating and/or administering said orodispersible particle.

Preferably, as referred to herein the carrier particles are templated carrier particles, preferably templated inverted particles, which also may be referred to as TIP particles. The technology of manufacturing and using TIP particles is described in detail in patent application PCT/EP2022/051799, which is incorporated herein by reference in its entirety.

Said templated inverted particles may also be referred to as carrier particles with secondary internal structure. As noted in PCT/EP2022/051799, the method for the production of carrier particles with secondary internal structures comprises the steps of a) combining a carrier material with a template material, wherein the carrier material forms a primary structure around the template material; b) transforming the template material; c) removing the transformed template material, and d) obtaining carrier particles with secondary internal structures.

It was surprisingly found that carrier particles exhibit the desired drug delivery properties when produced with a template material that undergoes a transformation as described herein.

Accordingly, whenever reference is made to carrier particles as described hereinabove, preferably the particles obtainable according to the method of production of carrier particles with secondary internal structure, as described hereinabove, are meant.

The term “primary structure” as used herein, refers to the layer of a carrier material that encompasses the template material. In some embodiments, the primary structure comprises further structure elements (e.g., petals as) that increase the surface area of the carrier particle.

The term “secondary internal structure”, as used herein, refers to a hollow internal structure, wherein the internal surface of the hollow internal structure is dense in crystallization initiation points. Therefore, the secondary internal structure enables crystallization inside the carrier particle.

The term “carrier material”, as used herein, refers to a material or a mixture that comprises the raw material for the carrier particle as described herein. In some embodiments, the carrier material described herein is an inorganic salt or comprises an inorganic salt to a substantial degree. In some embodiments, the carrier material described herein is insoluble or poorly soluble in water. In some embodiments, the carrier material is dissolved in a solvent. In some embodiments, the carrier material or a precursor of the carrier material is a liquid. In some embodiments, the carrier material described herein is a non-polymer or comprises a non-polymer to a substantial degree. The term “template material”, as used herein, refers to a solid material comprising particles suitable to serve as a template to enable the formation of the primary structure of the carrier particles. The particles in the template material preferably have the shape of a sphere, a spheroid, and/or a bead. In some embodiments, the template material described herein is a non-polymer or comprises a non-polymer to a substantial degree. In some embodiments, the template material described herein has a uniform or almost uniform particle size distribution. In some embodiments, the template material described herein has a distribution width (as defined by the formula: (D90−D10)/D50)) of about ≤5, about ≤4.5, about ≤4, about ≤3.5, about ≤3, about ≤2.8, about ≤2.4, about ≤2, about ≤1.8, about ≤1.6, about ≤1.4, about ≤1.2, about ≤1, about ≤0.9, about ≤0.8, about ≤0.7, about ≤0.6, about ≤0.5, about ≤0.4, about ≤0.3, about ≤0.2, or about ≤0.1. As such the template material is any material that is transformable and has sufficient stability to hold the carrier material. To avoid the dissolution of the template material during the step of combining a carrier material with a template material, a template material poorly soluble in a combining liquid should be used. In some embodiments, the template material described herein, is poorly soluble in at least one organic solvent selected from the group of dichloromethane, diethyl ether, toluene, ethanol, methanol, dimethyl sulfoxide, supercritical CO₂, dimethyl ketone, 2-propanol, 1-propanol, saturated alkanes, alkenes, alkadienes, fatty acids, glycerol, silicon oils, gamma-Butyrolactone, and tetrahydrofuran. In some embodiments, the template material described herein, is poorly soluble in water. In some embodiments, the template material described herein, is poorly soluble in an aqueous solution comprising solubility altering agents (e.g. salt water). In some embodiments, the term “poorly soluble” as described herein refers to a solubility at 25° C. of about <100 mg/L, <80 mg/L, <60 mg/L, <40 mg/L, <20 mg/L, <10 mg/L, <9 mg/L, <8 mg/L, <7 mg/L, <6 mg/L, <5 mg/L, <4 mg/L, <3 mg/L, <2 mg/L, <1 mg/L, <0.9 mg/L, <0.8 mg/L, <0.7 mg/L, <0.6 mg/L, <0.5 mg/L, <0.4 mg/L, <0.3 mg/L, <0.2 mg/L, <100 μg/L, <90 μg/L, <80 μg/L, <70 μg/L, <60 μg/L, <50 μg/L, <40 μg/L, <30 μg/L, <25 μg/L or <20 μg/L.

In some embodiments, the template material described herein comprises a salt. In some embodiments, the template material described herein comprises an organic salt. In some embodiments, the template material described herein is a carbonate salt or comprises a carbonate salt to a substantial degree. In some embodiments, the template material described herein comprises a basic oxide.

The term “transforming”, as used herein, refers to changing the properties of the template material by at least one physical step and at least one chemical step that in combination enable removal of the template material. The physical step of “transforming” comprises providing energy to the material. In some embodiments, the energy is applied in form of a rise in temperature, and/or alteration of pressure. In some embodiments, the physical step of “transforming” induces an endothermic chemical reaction in the template material. The chemical step of “transforming” comprises providing a chemical reactant to the template material. In some embodiments, the reactant provided in the chemical step of “transforming” reacts with the template material but not or not substantially with the carrier material. In some embodiments, the chemical reactant provided in the chemical step of “transforming” is provided in liquid, dissolved, and/or gaseous form.

Accordingly, the carrier particles as described herein are carrier particles with secondary internal structures. In some embodiments, these secondary internal structures enable high drug loading, because, without being bound by theory, the carrier particles can be loaded with the drug inside the secondary internal structures and not only on the surface of the carrier particles. The loaded agent or drug can leave the carrier by diffusion through the porous carrier wall. In some embodiments, the carrier particles have certain stability at a target site (e.g., on the mucosa of a patient). Therefore, these carrier particles can remain at a target site (e.g., by adhesion to the mucosa) and enable specific drug delivery. In some embodiments, the carrier particles mask the unpleasant taste of a loaded agent, because the loaded agent is continuously released at the site of absorption. The release rate of the loaded agent can be controlled by geometry of the template material and/or by diffusion rate modifiers such as disintegrants. Therefore, the unpleasant taste diffuses to a lesser extent to the locations of perceptions (e.g., the tongue).

The secondary internal structure described herein enables efficient drug loading on the inside of the carrier particle. Further, the secondary internal structure is accessible via pores e.g., for loading solvents. In some embodiments, the carrier particle can be loaded with less effort and/or has a particularly high loading capacity.

In some embodiments, the carrier particle has a particularly large surface area that is beneficial for interparticle forces. These interparticle forces act between the carrier particles in absence of water and increase the mechanical stability of carrier particle clusters. This increased mechanical stability reduces the need for additional stabilization material in the use of the carrier particles in pharmaceutical compositions such as solid pharmaceutical compositions, e.g., tablets. In some embodiments, the interparticle forces acting between the carrier particles can be diminished by water enabling a low disintegration time of pharmaceutical compositions such as solid pharmaceutical compositions, e.g., tablets, comprising the carrier particle as described herein.

In certain embodiments, the carrier material is an inorganic material or consists primarily of inorganic material.

The term “consists primarily of”, as used herein, in the context of a material refers to consisting of at least 50%, at least 60%, at least 70%, at least 80%, at least 90%, at least 95%, at least 96%, at least 97%, at least 98% or at least 99% of the material.

In certain embodiments, the carrier material and the template material are inorganic salts or consist primarily of inorganic salts.

The carrier particles as described herein with secondary internal structures that are beneficial to enhance one or more desired drug delivery properties.

In the process of producing said particles, the template material is preferably suspended in a liquid before combining a carrier material with a template material.

The template material can be suspended in a combining liquid (e.g., water) under stirring in a reaction vessel. The set agitation speed ensures stable turbulent mixing to impede particle agglomeration, which enables the treatment of the particles individually.

In certain embodiments, combining a carrier material with a template material comprises adding the template material described herein and the carrier material described herein to a combining liquid. In some embodiments, the combining liquid described herein is at least one organic solvent selected from the group of dichloromethane, diethyl ether, toluene, ethanol, methanol, dimethyl sulfoxide, supercritical CO₂, dimethyl ketone, 2-propanol, 1-propanol, saturated alkanes, alkenes, alkadienes, fatty acids, glycerol, silicon oils, gamma-Butyrolactone, and tetrahydrofuran. In some embodiments, the combining liquid described herein is water. In some embodiments, the combining liquid described herein is an aqueous solution comprising solubility altering agents (e.g. salt water).

To avoid dissolution of the template material during the step of combining a carrier material with a template material, an appropriate ratio of the amount of template material compared to the amount of the combining liquid should be used. This appropriate ratio depends on the solubility of the template material in the combining liquid. In some embodiments amount of the template material and combining liquid is chosen such that less than about 0.05% (w/w), less than about 0.04% (w/w), less than about 0.03% (w/w), less than about 0.02% (w/w), less than about 0.01% (w/w), less than about 0.0095% (w/w), less than about 0.009% (w/w), less than about 0.0085% (w/w), less than about 0.0008% (w/w), less than about 0.0075% (w/w), less than about 0.007% (w/w), less than about 0.0065% (w/w), less than about 0.06% (w/w), less than about 0.0055% (w/w), or less than about 0.005% (w/w) of the template material are dissolved in the combining liquid.

In certain embodiments, combining a carrier material with a template material comprises chemical precipitation, layering, and/or crystallization of the carrier material on the template material. The term “chemical precipitation”, as used herein, refers to the process of conversion of a chemical substance from a solution into a solid by converting the substance into an insoluble form.

In certain embodiments, combining a precursor of the carrier material forms the carrier material in a chemical reaction with the surface of the template material. In some embodiments, the soluble precursor of the carrier material described herein is phosphoric acid.

The conversion grade is relevant in embodiments wherein combining a precursor of the carrier material forms the carrier material in a chemical reaction with the surface of the template material. A too low conversion grade can cause particles with holes or broken shells, whereas a too high conversion can reduce the size of the inner cavity and produces more external crystals for example of dicalcium phosphate, which further converts to hydroxyapatite slabs. In some embodiments, the conversion grade described herein is between about 30% and about 60%, between about 35% and 55%, or between about 40% and about 50%.

The temperature during the chemical precipitation described herein can have a substantial influence on the material. For example, dicalcium phosphate as it is a less thermodynamically stable form than the hydroxyapatite. Therefore, too low temperatures and fast or uncontrolled orthophosphoric acid addition to calcium carbonate will trigger its precipitation and yield more dicalcium phosphate resulting in separate crystals that are more difficult to process. In some embodiments, the temperature during the chemical precipitation is about 60° C. or higher, preferably between about 60° C. and about 100° C., more preferably between about 70° C. and about 95° C., more preferably between about 80° C. and about 95° C.

In certain embodiments, a soluble precursor of the carrier material is added in a solution to the template material and distributed on the template material by the addition of a reactant that converts the soluble precursor of the carrier material to the insoluble carrier material. In some embodiments, the soluble precursor of the carrier material described herein is sodium phosphate or calcium chloride (e.g., as Despotović, R., et al., 1975, Calc. Tis Res. 18, 13-26).

The term “layering”, as used herein, refers to a technique for adding at least one layer of the carrier on the template material.

Any layering technique known in the art may be used (see, e.g., Decher, G. H. J. D., et al., 1992, Thin solid films, 210, 831-835; Donath, E., et al., 1998, Angewandte Chemie International Edition, 37 (16), 2201-2205; Caruso, F, et al., 1998, Science, 282(5391), 1111-1114). In some embodiments, electrostatic interactions (e.g., as described in Decher, G. H. J. D., et al., 1992, Thin solid films, 210, 831-835), hydrogen bonding (e.g., as described in Such, G. K. et al., 2010, Chemical Society Reviews, 40(1), 19-29), hydrophobic interactions (e.g., as described in Serizawa, T., Kamimura, S., et al., 2002, Langmuir, 18(22), 8381-8385), and/or covalent coupling (e.g., as described in Zhang, Y., et al., 2003, Macromolecules, 36(11), 4238-4240), electroplating and electrodeposition (e.g., as described in Chandran, R., Panda, S. K. & Mallik, A. A short review on the advancements in electroplating of CulnGaSe2 thin films. Mater Renew Sustain Energy 7, 6 (2018)) are exploited to prepare at least one layer on the template material, particularly to prepare multilayered films on the template material.

The term “crystallization”, as used herein, refers to the process of conversion of a chemical substance from a super-saturated solution.

In certain embodiments, the carrier material is added in a super-saturated solution to the template material and distributed on the template material by the initiation of chemical precipitation.

In certain embodiments, combining a carrier material with a template material comprises chemical precipitation and crystallization of the carrier material on the template material.

In certain embodiments, combining a carrier material with a template material comprises chemical layering and crystallization of the carrier material on the template material.

In certain embodiments, combining a carrier material with a template material comprises chemical precipitation and layering of the carrier material on the template material.

The chemical precipitation process can be carried out by pumping a solution of a precursor of the template material onto the carrier material or into the liquid comprising the carrier material. During this process, the carrier material can start growing (e.g., in the form of a crystalline lamellae structure) on the surface of template material and thus forming the stratum layer. In certain embodiments, the template material as described herein is converted to the carrier material. In certain embodiments, the template material as described herein is converted to at least about 20%, about 30%, about 40%, about 50%, about 60%, or about 70% to the carrier material.

Chemical precipitation, layering, and/or crystallization enable fine and/or uniform distribution of the carrier material on the template material. This fine and/or uniform distribution affects the formation of the secondary internal structures.

Accordingly, the carrier particles produced as described herein exhibit particularly fine and/or uniform secondary internal structures by using chemical precipitation, layering, and/or crystallization of the carrier material on the template material.

In certain embodiments, transforming the template material comprises heating to a temperature from about 600° C. to about 1200° C., preferably about 600 to about 900° C., preferably about 600° C. to 839° C., preferably about 650° C. to about 700° C.

In certain embodiments, transforming the template material comprises heating to a temperature from 840° C. to 1200° C.

The conditions can be optimized to avoid interparticle condensation during the heating step, which can result in redispersability problems. While in some embodiments no further agents to avoid interparticle condensation need to be added, in other embodiments agents to avoid interparticle condensation (e.g., anti-sintering agents) are added during and/or before the heating step described herein. Such anti-sintering agents are described for example in Okada, M., et al., 2014, Journal of nanoparticle research, 16(7), 1-9.

The transformation of the template material described herein can be done at any suitable temperature or any suitable temperature range. To enable the transformation of the template material described herein the minimal suitable temperature for transformation is set at a certain temperature e.g., about 210° C. (e.g., for silver and gold carbonate as the template material), about 840° C. (e.g., for calcium carbonate as the template material), about 900° C., about 1000° C., or about 1200° C. (e.g., for potassium and/or sodium carbonates as template material). The person skilled in the art can identify the appropriate minimal suitable temperature from the decomposition temperature of the template material. An increased temperature can shorten the transformation time, however, melting of the carrier material may have an undesired effect on the carrier particles such as incomplete carrier particle formation or reduced carrier particle hardness. To avoid melting of the carrier material, the maximal suitable temperature for the transformation of the template material described herein is set below the melting temperature of the carrier material. Deformation and/or loss of desired structures (e.g., petals on the surface of the carrier particles) that enhance the surface area of the carrier particles can already occur at temperatures below the melting temperature of the carrier material. Accordingly, in certain embodiments, the maximal suitable temperature for the transformation of the template material described herein is set about 100° C., about 200° C., about 400° C., about 500° C., or about 600° C. below the melting temperature of the carrier material.

In certain embodiments, transforming the template material comprises heating to a temperature from about the decomposition temperature of the template material to about the melting temperature of the carrier material, preferably from about the decomposition temperature of the template material to about 400° C. below the melting temperature of the carrier material, more preferably about the decomposition temperature of the template material to about 500° C. below the melting temperature of the carrier material.

In certain embodiments, transforming the template material comprises heating to a temperature from 840° C. to 1600° C., preferably from 840° C. to 1200° C., more preferably around 1100° C.

The duration of the heating for transforming the template material described herein depends on various factors such as the template material, the carrier material, the temperature range, particle size, and/or the desired carrier particle surface area.

The duration of the heating for transforming the template material described herein may for example be about 1 hour. In certain embodiments, the duration of the heating for transforming the template material described herein is between about 5 min and about 24 h, about 10 min and about 12 h, 20 min and about 4 h.

The heating for transforming the template material described herein (e.g., to a temperature in a certain range, e.g., between 840° C. to 1200° C. or 600° C. to 900° C.) can be achieved by any heating pattern such as a linear increase of temperature or with one or more preheating steps. The preheating steps described herein may comprise keeping the temperature at a certain temperature level for a certain time before heating the template material to a temperature in a certain range, e.g., from 840° C. to 1200° C. or 600° C. to 900° C. Preheating allows for example removal of undesired volatile components such as solvents.

In some embodiments, the pressure is reduced during the heating for transforming the template material to a temperature in a certain range, e.g., from 840° C. to 1200° C.

In some embodiments, the pressure is increased during the heating for transforming the template material to a temperature in a certain range, e.g., from 840° C. to 1200° C.

In some embodiments, the heating for transforming the template material induces an endothermic chemical reaction.

In some embodiments, an inert substance (e.g., noble gas) is supplied to avoid side reactions during the heating for the transforming the template material to a temperature in a certain range, e.g., from 840° C. to 1200° C.

In some embodiments, the heating for transforming the template material induces the evaporation of volatile fractions of the template material.

The heating to a temperature in a certain range, e.g., from 840° C. to 1200° C., may initiate the transformation of the template material but does not or not to the same extent alter the carrier material. This enables the removal of the transformed template material based on the altered properties. Lower temperature (e.g. about 600° C. to about 839° C. or 600° C. to about 900° C.) can be used to maintain the petals' structure to a larger degree, which can increase the resulting tablet hardness.

In case the temperatures are higher than the recommended range, the fine petal structure of the particles is molten and is reduced, the flexibility of the petals is reduced; therefore, the hardness of the tablets produced with such overheated material is strongly reduced. Pharmaceutical compacts made with overheated material show capping and lamination and cannot be used comparably well in pharmaceutical formulations.

Accordingly, a heating step for the transformation of the template material enables the production of carrier particles with secondary internal structures that are beneficial to enhance one or more desired drug delivery properties.

In certain embodiments, the step of transforming the template material comprises calcination.

The term “calcination”, as used herein, refers to heating a solid or a mixture comprising a solid to high temperatures (e.g., a temperature from 840° C. to 1200° C. or 600° C. to 900° C.) under the supply of air or oxygen to the solid or the mixture.

In some embodiments, the calcination as described herein induces decomposition of template material comprising a carbonate (e.g., carbonate salts such as calcium carbonate) to carbon dioxide.

In some embodiments, the calcination as described herein induces decomposition of template material comprising a metallic carbonate to a metallic oxide, preferably to a basic oxide.

In some embodiments, the calcination as described herein induces the decomposition of hydrated template material by the removal of water.

In some embodiments, the calcination as described herein induces the decomposition of volatile matter in the template material.

Accordingly, the calcination step for the transformation of the template material enables the production of carrier particles with secondary internal structures that are beneficial to enhance one or more desired drug delivery properties.

In certain embodiments, transforming the template material comprises a subsequent addition of water.

The subsequent addition of water transforms the template material in a chemical reaction but does not alter or unsubstantially alter the carrier material. This enables the removal of the transformed template material based on the altered properties.

In some embodiments, the subsequent addition of water as described herein reacts with a metallic oxide.

Accordingly, the transformation step method comprises the addition of water enables the production of carrier particles with secondary internal structures that are beneficial to enhance one or more desired drug delivery properties.

In certain embodiments, the addition of water enables an exothermic reaction.

The term “exothermic reaction”, as used herein, refers to a reaction for which the overall standard enthalpy change is negative.

The subsequent addition of water as described herein transforms the template material in an exothermic chemical reaction but does not alter or unsubstantially alter the carrier material. This enables the removal of the transformed template material based on the altered properties.

The basic oxide described herein, is not toxic or unsubstantially toxic at the dose used as described herein. In some embodiments, the subsequent addition of water as described herein reacts with a basic oxide. In some embodiments, the subsequent addition of water as described herein reacts with at least one basic oxide selected from the group of lithium oxide, sodium oxide, potassium oxide, rubidium oxide, cesium oxide, magnesium oxide, calcium oxide, strontium oxide, barium oxide, and bismuth (III) oxide. In some embodiments, the subsequent addition of water as described herein reacts with magnesium oxide and/or calcium oxide.

The exothermic reaction as described herein can facilitate subsequent removal of the template material. The forces released during the exothermic reaction and/or the properties of the products of the exothermic reaction can decrease density and/or increase solubility. For example, the exothermic reaction of calcium oxide with a density of 3.34 g/cm³ with water results in calcium hydroxide with a density of 2.21 g/cm³.

Accordingly, the addition of water through an exothermic reaction supports the secondary structure formation and facilitates subsequent template material removal.

In certain embodiments, removing the template material comprises dissolution of the transformed template material to form secondary internal structures.

The secondary internal structures can be formed by the removal of the transformed template material by dissolution in a solvent that dissolved the transformed template material but not the carrier material.

In some embodiments, removing the template material comprises dissolution of the transformed template material with water or an aqueous solution. In some embodiments, the pH of the aqueous solution is altered before the dissolution of the transformed template material to increase the solubility of the transformed template material or decrease the solubility of the carrier material in the aqueous solution.

In some embodiments, removing the template material comprises the dissolution of the transformed template with an organic solvent.

The removal of the template material by dissolution is particularly mild to the carrier material. Therefore, this mild removal supports the maintenance of the primary carrier material structure and enables the formation of secondary internal structures that are particularly beneficial for crystallization during the drug loading process.

Accordingly, removing the template material comprises dissolution of the transformed template material supports the formation of the secondary internal structures.

In certain embodiments, the template material comprises a metal carbonate.

In certain embodiments, the template material comprises at least one metal carbonate selected from the group of Li₂CO₃, LiHCO₃, Na₂CO₃, NaHCO₃, Na₃H(CO₃)₂, MgCO₃, Mg(HCO₃)₂, Al₂(CO₃)₃, K₂CO₃, KHCO₃, CaCO₃, Ca(HCO₃)₂, MnCO₃, FeCO₃, NiCO₃, Cu₂CO₃, CuCO₃, ZnCO₃, Rb₂CO₃, PdCO₃, Ag₂CO₃, Cs₂CO₃, CsHCO₃, BaCO₃, and (BiO)₂CO₃.

In certain embodiments, the template material comprises at least one metal selected from the group of Fe, Mg, Al, Mn, V, Ti, Cu, Ga, Ge, Ag, Au, Sm, U, Zn, Pt and Sn. In certain embodiments, the template material comprises at least one non-metal selected from the group of Si, S, Sb, I, and C.

In certain embodiments, the template material comprises more than 50%, 60%, 70%, 80%, 90%, 95%, 96%, 97%, 98% or 99% metal carbonate.

In certain embodiments, the template material comprises more than 50%, 60%, 70%, 80%, 90%, 95%, 96%, 97%, 98% or 99% of at least one metal carbonate selected from the group of Li₂CO₃, LiHCO₃, Na₂CO₃, NaHCO₃, Na₃H(CO₃)₂, MgCO₃, Mg(HCO₃)₂, Al₂(CO₃)₃, K₂CO₃, KHCO₃, CaCO₃, Ca(HCO₃)₂, MnCO₃, FeCO₃, NiCO₃, Cu₂CO₃, CuCO₃, ZnCO₃, Rb₂CO₃, PdCO₃, Ag₂CO₃, Cs₂CO₃, CsHCO₃, BaCO₃, and (BiO)₂CO₃.

In certain embodiments, the template material comprises more than 50%, 60%, 70%, 80%, 90%, 95%, 96%, 97%, 98% or 99% magnesium carbonate.

In certain embodiments, the template material comprises calcium carbonate.

In certain embodiments, the template material comprises more than 50%, 60%, 70%, 80%, 90%, 95%, 96%, 97%, 98% or 99% calcium carbonate.

In some embodiments, the calcium carbonate as described herein comprises anhydrous calcium carbonate, complexes comprising calcium carbonate and/or hydrated calcium carbonate such as CaCO₃·H₂O and/or calcium carbonate hexahydrate.

In some embodiments, the calcium carbonate as described herein is anhydrous calcium carbonate.

The metal carbonates described herein can be used as a basis to produce a carrier material with distinct properties (e.g., an insoluble metal phosphate by a reaction of the metal carbonate with H₃PO₄) on the surface of the template material and can be transformed as described herein.

In certain embodiments, the carrier material comprises at least one salt and/or complex selected from the group of calcium phosphate and magnesium phosphate.

In certain embodiments, the carrier material comprises at least one salt and/or complex of magnesium phosphate.

In certain embodiments the carrier material comprises at least one salt and/or complex of calcium phosphate.

Calcium phosphate and magnesium phosphate have a particularly low solubility in water and show a reasonable heat resistance. Furthermore, calcium phosphate and magnesium phosphate are typically pharmacologically inert and non-toxic. Therefore, calcium phosphate and magnesium phosphate are robust, non-toxic, and allow the transformation of the template material as described herein without decomposition.

Accordingly, the production of the carrier particles as described herein is particularly efficient when the carrier material comprises at least one salt and/or complex selected from the group of calcium phosphate and magnesium phosphate.

Preferably, the carrier particles as encompassed by the present invention comprise calcium phosphate and/or magnesium phosphate. More preferably, the carrier particles as encompassed by the present invention comprise calcium phosphate.

Preferably, the calcium phosphate is present in the form of hydroxyapatite. As referred to herein, hydroxyapatite is a substance according to formula Ca₅(OH)(PO₄)₃

Accordingly and preferably, the carrier particles as encompassed by the present invention comprise hydroxyapatite. Further preferably, the carrier particles as encompassed by the present invention further comprise calcium hydroxide.

Thus, preferably, the present invention relates to an embodiment, wherein the compositions, the salts and parts of the kits of parts of the present invention may be formulated by using carrier particles with secondary internal structures, wherein said carrier particles comprise hydroxyapatite and optionally comprise calcium chloride.

Preferably, the content of the hydroxyapatite in said particle (not loaded with the composition, the salt or part(s) of the kits of parts of the present invention) is at least 80% w/w, preferably at least 90% w/w, more preferably at least 95% w/w, even more preferably at least 99% w/w, even more preferably about 100% w/w.

As preferably it is to be understood herein, the term “carrier particles with secondary internal structures” may also be referred to as “carrier particles with hollow internal structures”.

The template material can have various structures, e.g., powder (e.g., a powder with D50 of about: 1.9 μm, 2.3 μm, 3.2 μm, 4.5 μm, 5.5 μm, 6.5 μmo or 14 μm; a powder with a particle size range of about: 1 to 100 μm, 100 μm to 300 μm or 300 μm to 600 μm) or nanoparticles.

In certain embodiments, the template material comprises particles that have a diameter of 1 to 300 μm. In certain embodiments, the template material consists of particles wherein about 50%, about 60%, about 70%, about 80%, about 90%, about 95%, about 98%, about 99% of the particles that have a diameter of 1 to 300 μm. In certain embodiments, the template material comprises particles that have a median diameter of about 1 to 300 μm, about 1 to 250 μm, about 1 to 200 μm, about 1 to 150 μm, about 1 to 100 μm, about 1 to 90 μm, about 1 to 80 μm, about 1 to 70 μm, about 1 to 60 μm, about 1 to 50 μm, about 1 to 40 μm, about 1 to 30 μm or about 1 to 20 μm.

The particle size of the template material influences the diameter of the carrier particle. In certain embodiments, the particles of the template material have about the same median diameter as the median diameter of the carrier particles. In embodiments wherein the template material and the carrier material are combined by layering and/or crystallization as described herein, the carrier particle has a similar or larger median diameter compared to the template material.

In embodiments wherein the template material and the carrier material are combined by chemical precipitation as described herein, the carrier particle has a similar or smaller median diameter compared to the template material.

The person skilled in the art can predict the carrier material from the template material, carrier material, and the techniques used for combining the template material with the carrier material as described herein.

In certain embodiments, the carrier particles have a diameter of 1 to 300 μm.

Particles of a certain size can be obtained by methods known in the art, including milling, sieving (see, e.g., Patel, R. P., et al., 2014, Asian Journal of Pharmaceutics (AJP), 2(4); DAVID, J., and PETER, R., 2006, Fundamentals of Early Clinical Drug Development: From Synthesis Design to Formulation, 247; U.S. Pat. No. 5,376,347A). Particle size and shape measurements can be made using any method known in the art, such as laser diffraction or in situ microscopy (Kempkes, M., Eggers, J., & Mazzotti, M., 2008, Chemical Engineering Science, 63(19), 4656-4675; Allen, T. (2013). Particle size measurement. Springer).

In some applications, a particular low carrier particle diameter is desired. In certain embodiments, the carrier particles have a diameter of about 1 to 20 μm, about 1 to 15 μm, about 1 to 10 μm, or about 1 to 5 μm for use in intrapulmonary administration and/or nasal administration. In some applications, a particular low carrier particle diameter is desired to increase the diffusion surface and accelerate the release of the loaded agent.

In some applications, a larger carrier particle diameter is desired to enhance the flowability of the carrier particles and to facilitate further processing. In certain embodiments, the carrier particles have a diameter of about 5 to 300 μm, about 10 to 250 μm, about 15 to 200 μm, or about 20 to 150 μm.

Accordingly, the method for the production of the carrier particles as described herein wherein the carrier particles have a diameter in a certain range can be particularly useful for further processing (e.g., flowability) and/or application (e.g., diffusion surface) of the carrier particle produced according to said method.

In certain embodiments, the carrier particles have a surface area between 15 m2/g to 400 m2/g or 30 m2/g to 400 m2/g.

In certain embodiments, the carrier particles have a surface area between about 15 m2/g to 400 m2/g about 30 m2/g to 400 m2/g, about 50 m2/g to 350 m2/g, about 70 m2/g to 320 m2/g, about 90 m2/g to 300 m2/g or about 100 m2/g to 280 m2/g as measured by 5-point BET (Brunnauer-Emmet-Teller) surface area analysis with nitrogen as a gas.

Alternatively, the surface area of carrier particles can be measured by any method known in the art (see, e.g., Akashkina, L. V., Ezerskii, M. L., 2000, Pharm Chem J 34, 324-326; Bauer, J. F., 2009, Journal of Validation Technology, 15(1), 37-45).

The surface area of the carrier particles can be altered e.g., by the particle size of the carrier material, the carrier material, and/or changing the surface structure by the parameters as described herein (e.g., heat, duration of heating).

In certain embodiments the carrier particle is used as an adsorber.

A greater specific surface of carrier particles described herein allows strong Van der Waals interactions once the particles are brought in contact. This effect results in higher tensile strength of the final dosage forms. These Van der Waals interactions can be diminished by the addition of water and support the disintegration of particle clusters.

Accordingly, the method for the production of carrier particles as described herein enables mechanical stability and disintegration capabilities if the carrier particles have a surface area between 15 m2/g to 400 m2/g, preferably 30 m2/g to 400 m2/g.

In certain embodiments, the secondary internal structure comprises pores having a diameter size in the range of ≥0.2 μm and ≤1.5 μm.

In certain embodiments the secondary internal structure comprises pores having a diameter size of about ≥0.2 μm, about ≥0.3 μm, about ≥0.4 μm, about ≥0.5 μm, about ≥0.6 μm, about ≥0.7 μm, about ≥0.8 μm, about ≥0.9 μm, about ≥1 μm, about ≥1.1 μm, about ≥1.2 μm, about ≥1.3 μm, or about 1.5 μm.

In certain embodiments the secondary internal structure comprises pores having a diameter size in the range of about ≥0.2 μm to ≤1.5 μm, about ≥0.3 μm to ≤1.5 μm, about ≥0.4 μm to ≤1.5 μm, about ≥0.5 μm to ≤1.5 μm, about ≥0.6 μm to ≤1.5 μm, about ≥0.7 μm to ≤1.5 μm, about ≥0.8 μm to ≤1.5 μm, about ≥0.9 μm to ≤1.5 μm, about ≥1 μm to ≤1.5 μm, about ≥1.1 μm to ≤1.5 μm, about ≥1.2 μm to ≤1.5 μm or about ≥1.3 μm to ≤1.5 μm.

The pore size of carrier particles can be measured by any method known in the art (see, e.g. Markl, D. et al., 2018, International Journal of Pharmaceutics, 538(1-2), 188-214).

The porous structure that can be formed by the method for the production of the carrier particles as described herein enables pores of a, particularly, large size. This large pore size facilitates drug loading on the carrier particle and accelerates drug release from the carrier particle.

A pore size diameter greater than 90% of the diameter of the particles of the template material results in unstable carrier particles. Therefore, the maximal pore size depends on the size particles of the template material.

In certain embodiments, the secondary internal structure comprises pores having a diameter size of about ≤270 μm, about ≤225 μm, about ≤180 μm, about ≤135 μm, about ≤90 μm, about ≤81 μm, about ≤72 μm, about ≤63 μm, about ≤54 μm, about ≤45 μm, about ≤36 μm, about ≤27 μm, or about ≤18 μm diameter. Accordingly, the method for the production of the carrier particles as described herein, wherein the secondary internal structure comprises pores that have a certain diameter size is particularly useful for the subsequent drug loading and drug release of the carrier particles produced as described herein.

In certain embodiments, the total volume of the secondary internal structures in the obtained carrier particles with secondary internal structures is in the range of ≥10% to ≤90% of the particle volume as determined by image analysis of SEM-FIB and SEM of resin-embedded particles' cross-section images. Alternative analytical methods to measure the volume ratio of the internal structure and particle include porosity calculation as a ratio of tapped bulk of the carrier material to the true crystalline density of the carrier material.

The total volume of the secondary internal structures refers to the volume inside the particle inside that results from the removal of the template material. In certain embodiments, the total volume of the secondary internal structures described herein is the average internal volume of the carrier particles obtained as described herein.

In certain embodiments, the total volume of the secondary internal structures described herein is the median internal volume of the carrier particles obtained as described herein.

In certain embodiments, the total volume of the secondary internal structures in the obtained carrier particles with secondary internal structures is more than about 10%, about 15%, about 20%, about 25%, about 30%, about 35%, about 40%, about 50%, about 60%, about 70%, or about 80% of the particle volume.

In certain embodiments, the total volume of the secondary internal structures in the obtained carrier particles with secondary internal structures is more than about 10%, about 15%, about 20%, about 25%, about 30%, about 35%, about 40%, about 50%, about 60%, about 70%, or about 80% of the particle volume.

In certain embodiments, the total volume of the secondary internal structures in the obtained carrier particles with secondary internal structures is in the range of about ≥10%-≤90%, about ≥15%-≤90%, about ≥20%-≤90%, about ≥25%-≤90%, about ≥30%-≤90%, about ≥35%-≤90%, about ≥40%-≤90%, about ≥45%-≤90%, about ≥50%-≤90%, about ≥55%-≤90%, about ≥60%-≤90%, about ≥65%-≤90%, about ≥70%-≤90%, about ≥10%-≤80%, about ≥15%-≤80%, about ≥20%-≤80%, about ≥25%-≤80%, about ≥30%-≤80%, about ≥35%-≤80%, about ≥40%-≤80%, about ≥45%-≤80%, about ≥50%-≤80%, about ≥55%-≤80%, about ≥ 60%-≤80%, about ≥65%-≤80%, about ≥70%-≤80%, about ≥10%-≤70%, about ≥15%-≤70%, about ≥20%-≤70%, about ≥25%-≤70%, about ≥30%-≤70%, about ≥35%-≤70%, about ≥40%-≤70%, about ≥45%-≤70%, about ≥50%-≤70%, about ≥55%-≤70%, about ≥60%-≤70%, about ≥65%-≤70%, about ≥10%-≤60%, about ≥15%-≤60%, about ≥20%-≤60%, about ≥25%-≤60%, about ≥30%-≤60%, about ≥35%-≤60%, about ≥40%-≤60%, about ≥45%-≤60%, about ≥ 50%-≤60%, about ≥55%-≤60%, about ≥10%-≤50%, about ≥15%-≤50%, about ≥20%-≤50%, about ≥25%-≤50%, about ≥30%-≤50%, about ≥35%-≤50%, about ≥40%-≤50% or about ≥45%-≤50% of the particle volume.

In certain embodiments of the carrier particle as described herein and obtainable as described hereinabove, the carrier particle has a loading capacity of ≥72% v/v, ≥70% v/v, ≥68% v/v, ≥66% v/v, ≥64% v/v, ≥62% v/v, or ≥60% v/v.

In certain embodiments of the carrier particle as described herein, the carrier particle has a loading capacity of ≥60% v/v.

The term “loading capacity”, as used herein, refers to the volume of the carrier particle that can be used for loading of an agent compared to the volume of the whole carrier particle. Accordingly, a carrier particle with a loading capacity of 60% v/v can load an agent having 60% of the volume of the carrier particle. The volume of the carrier particle is calculated from the diameter of the carrier particle. Therefore, the volume of the internal structure is part of the volume of the carrier particle for this calculation.

In some embodiments, an agent that is loaded on the carrier particle is comprised of a loading solvent and the loading solvent is removed to complete loading.

The agent to be loaded is dissolved in the loading solvent and put in contact with the carrier particle ensuring complete wetting of the latter. The loading solvent can be removed by method any solvent removal method known to the person skilled in the art.

In some embodiments the loading solvent is removed by a method selected from the group of evaporation, vacuum-assisted evaporation, atmospheric drying, vacuum-freeze drying, freeze drying at atmospheric pressure, spray drying, spray drying in fluidized bed apparatus, microwave assisted drying, electrospray-assisted drying, dielectric drying, fluidized-bed assisted drug loading, and solvent-sorption method.

In the present invention, the agent to be loaded in the carrier particle is the composition, the salt or the part(s) of the kits of parts of the present invention.

In some embodiments, the solvent-sorption method comprises high shear granulation.

The choice of the appropriate loading solvent depends on solvent toxicity, solvent partial vapor pressure, properties of the agent to be loaded (e.g., pH-stability and/or solubility of the agent to be loaded) and/or properties of the carrier material.

In some embodiments, the loading solvent described herein comprises at least one organic solvent, preferably at least one organic solvent selected from the group of dichloromethane, diethyl ether, toluene, ethanol, methanol, dimethyl sulfoxide, supercritical CO₂, dimethyl ketone, 2-propanol, 1-propanol, saturated alkanes, alkenes, alkadienes, fatty acids, glycerol, silicon oils, gamma-Butyrolactone, and tetrahydrofuran. In some embodiments, the loading solvent described herein is water.

Some loading solvents such as water have high surface tension and may therefore require additional measures to support entering the pore(s) of the carrier particle as described herein despite the exceptionally large pore size. In some embodiments, the loading solvent described herein comprises at least one surface-active agent such as a tenside. In some embodiments, the addition of the loading solvent occurs under increased pressure, to support the loading solvent by entering into the inside of the carrier particle.

In some embodiments, loading on and into the carrier particle as described herein comprises the addition of an antisolvent that reduces the solubility of the agent to be loaded in the loading solvent. In some embodiments, the antisolvent is at least one antisolvent selected from the group of water, dichloromethane, diethyl ether, toluene, ethanol, methanol, dimethyl sulfoxide, supercritical CO₂, dimethyl ketone, 2-propanol, 1-propanol, saturated alkanes, alkenes, alkadienes, fatty acids, glycerol, silicon oils, gamma-Butyrolactone, and tetrahydrofuran.

In some embodiments, the loading solvent is removed by evaporation, e.g., by increased temperature and/or decreased pressure. The maximal temperature for the removal of the loading solvent depends on the heat stability of the loaded agent.

The carrier particles with secondary internal structures, as described herein, can be compacted to obtain compacted carrier particles.

The term “compacted carrier matter”, as used herein, refers to clusters of more than one carrier particle with adhesive forces acting between the carrier particles.

The term “compacting”, as used herein, refers to applying pressure to more than one particle (e.g., carrier particle) to form compacted carrier matter, wherein the carrier particle at least partially remains adherent to each other upon release of the pressure. Techniques for compacting are known to the person skilled in the art (see, e.g., Odeku, O. A. et al., 2007, Pharmaceutical Reviews, 5(2)). Examples of techniques for compaction include, without limitation tableting, roller compaction, slugging, briquetting and/or centrifugation.

The compacted carrier matter described herein is particularly stable and can be used for the obtainment of a particularly stable pharmaceutical composition. During compaction, the large surface areas of the carrier particles as described herein form strong interparticle Van Der Waals adhesion forces that enable mechanical stability. Upon intake, water enters between the particles (e.g., by capillary forces), the distance-dependent Van Der Waals adhesion forces diminish, and the compacted carrier matter disintegrates.

Accordingly, the compacted carrier matter described herein show particular mechanical stability and/or fast disintegration time.

It has been surprisingly found by the present inventors that the formulations of the compositions, the salts and parts of the kits of parts of the present invention formulated using carrier particles show improved bioavailability and/or reduced bitter taste, thereby leading to increased compliance with the patients.

Accordingly and preferably, the carrier particles as described in the present invention are compacted.

Thus, preferably, the present invention relates to an embodiment, wherein the compositions, the salts and parts of the kits of parts of the present invention may be formulated by using carrier particles with secondary internal structures (which may also be referred to as hollow internal structures), wherein the carrier particles are compacted, wherein said carrier particles comprise hydroxyapatite and optionally comprise calcium chloride. Preferably, the content of the hydroxyapatite in said particle (not loaded with the composition, the salt or the part(s) of the kits of parts of the present invention) is at least 80% w/w, preferably at least 90% w/w, more preferably at least 95% w/w, even more preferably at least 99% w/w, even more preferably about 100% w/w.

It has been surprisingly found by the present inventors that formulations of harmine according to the present invention, in particular harmine glucuronate, when formulated by using carrier particles with hollow internal structures (carrier particles with secondary internal structures) do not lead to bitter taste upon administration into oral cavity. Accordingly, by formulating the compositions, the salts, the pharmaceutical compositions of the present invention or the parts of kits of parts of the present invention, masking of the bitter taste of harmine or its salt is achieved (see Part 8 in Examples). In turn, the skilled person will appreciate that this may lead to improved compliance.

Accordingly, the invention further relates to a method for masking the bitterness of a compound, wherein the compound is harmine or a pharmaceutically acceptable salt thereof, or DMT or a pharmaceutically acceptable salt thereof, the method comprising loading a compound having a bitter taste onto a carrier particle wherein a) the carrier particle comprises a loading cavity and wherein the carrier particle comprises a basic salt; and b) wherein the bitterness of the compound is masked by the carrier particle during oral mucosal absorption.

The inventors found that a carrier particle comprising a basic salt and a loading cavity can be used to mask taste, such as bitterness, of a compound, the compound being DMT, harmine or their salts, and that this masking effect goes beyond the masking properties of the geometric form of the carrier particle for certain compounds. Without being bound by theory, the basic salt may turn a part of the loaded compound to a tasteless form (e.g., the freebase or non-salt form of the loaded compound), which may then embody a film or barrier to shield the compound from being perceived as having a certain taste, e.g. by shielding the compound from the tastebuds. As such, the combination of chemical and structural shielding can mask tastes surprisingly well while also providing improved drug delivery properties. This method can be applied to any taste, preferably to a quantifiable taste such as bitterness.

This method may be used to improve compliance of a subject to a therapy (e.g. a therapy with a bad taste) or to improve oral mucosal absorption by improving tolerability of the compound in the mouth.

Accordingly, the invention further relates to a pharmaceutical composition comprising carrier particles, comprising a) a carrier particle comprising a loading cavity and comprising of a basic salt; and b) a compound having a bitter taste, wherein the compound is harmine or a pharmaceutically acceptable salt thereof, or DMT or a pharmaceutically acceptable salt thereof, wherein the bitterness of the compound is masked by the carrier particle during oral mucosal absorption. It is to be understood that the compound having a bitter taste, as described herein, may have a bitter taste in its salt form but no bitter taste or a reduced bitter taste in its non-salt form. The particle therefore enables the processing of the salt form instead of the non-salt form can for example facilitate loading of particles and/or tablet production.

The basic salt is not necessarily the primary constituent of the carrier particle. The basic salt can also only be present in small amounts (e.g. below the detection limit of certain measurement methods) for example residues from production, as long as it is sufficient to react with the loading agent in a sufficient amount to mask the taste. In certain embodiments, the basic salt is calcium hydroxide and/or magnesium hydroxide.

In certain embodiments, the invention relates to the method of the invention or the pharmaceutical composition comprising carrier particles of the invention, wherein the basic salt is calcium hydroxide.

In certain embodiments, the invention relates to the method of the invention or the pharmaceutical composition comprising carrier particles of the invention, wherein the carrier particle comprises a porous hydroxyapatite shell, at least one hollow cavity and calcium hydroxide.

In certain embodiments, the invention relates to the method of the invention or the pharmaceutical composition comprising carrier particles of the invention, wherein the carrier particle comprises a porous hydroxyapatite shell, at least one hollow cavity and calcium hydroxide, wherein the calcium hydroxide is present in a smaller amount than the hydroxyapatite, preferably wherein the amount of calcium hydroxide is at least 2 times, at least 5 times, at least 10 times, at least 50 times or at least 100 times smaller than the amount of hydroxyapatite.

In certain embodiments, the invention relates to the method of the invention or the pharmaceutical composition comprising carrier particles of the invention, wherein the carrier particle is obtainable or obtained by the steps of: a) combining a carrier material with a template material, wherein the carrier material forms a primary structure around the template material; b) transforming the template material; c) removing the transformed template material; and d) obtaining carrier particles with secondary internal structures.

In certain embodiments, the invention relates to a method for the production of carrier particles with secondary internal structures comprising the steps of a) combining a carrier material with a template material, wherein the carrier material forms a primary structure around the template material; b) transforming the template material; c) removing the transformed template material, and d) obtaining carrier particles with secondary internal structures.

It is to be understood that the carrier particles used in a method for masking the bitterness of a compound of the invention or in the pharmaceutical composition comprising carrier particles of the invention are as described hereinabove, and can be obtained as described hereinabove.

Preferably, the pharmaceutical composition comprising carrier particles of the invention is a solid pharmaceutical composition, preferably a solid pharmaceutical composition for oral, sublingual, buccal, nasal, bronchial, rectal, urethral, and/or intravaginal administration, more preferably for oral, sublingual or buccal administration.

Tablets, capsules or sachets for peroral administration are usually supplied in dosage units and may contain conventional excipients, such as binders, fillers, diluents, tableting agents, lubricants, detergents, disintegrants, colorants, flavors and wetting agents. Tablets may be coated in accordance to methods well known in the art. Suitable fillers include or are preferably cellulose, mannitol, lactose and similar agents. Suitable disintegrants include or are preferably starch, polyvinyl pyrrolidone and starch derivatives such as sodium starch glycolate. Suitable lubricants include or are preferably, for example, magnesium stearate. Suitable wetting agents include or are preferably sodium lauryl sulfate. These solid oral compositions can be prepared with conventional mixing, filling or tableting methods. The mixing operations can be repeated to disperse the active agent in compositions containing large quantities of fillers. These operations are conventional.

The parts of the kit of parts of the invention may be prepared by mixing suitably selected and pharmaceutically acceptable excipients, vehicles, adjuvants, additives, surfactants, desiccants or diluents known to those well-skilled in the art, and can be suitably adapted for oral, parenteral or topical administration. Typically and preferably the parts of the kit of parts of the invention is administered in the form of a tablet, capsule, sachets, powder, granule, pellet, orodispersible tablet, mucoadhesive film, lyophilizate, oral or parenteral solution, suspension, suppository, ointment, cream, lotion, gel, paste and/or may contain liposomes, micelles and/or microspheres.

The parts of the kit of parts or the pharmaceutical composition of the present invention as liquid compositions for oral administration can be provided in the form of, for example, aqueous solutions, emulsions, syrups or elixirs or in the form of a dry product to be reconstituted with water or with a suitable liquid carrier at the time of use. The liquid compositions can contain conventional additives, such as suspending agents, for example sorbitol, syrup, methylcellulose, gelatin, hydroxyethyl cellulose, carboxymethyl cellulose, aluminum stearate gel or hydrogenated edible fats; emulsifying agents, for example lecithin, sorbitan monooleate, or acacia; non aqueous carriers (which can include edible oil), for example almond oil, fractionated coconut oil, oily esters, such as glycerin esters, propylene glycol or ethyl alcohol; preservatives, for example methyl or propyl p-hydroxybenzoate or sorbic acid; penetration enhancer, for example dimethylsulfoxide (DMSO); pH buffer systems, for example phosphate buffer, carbonate buffer, citrate buffer, citrate-phosphate buffer and other pharmaceutically acceptable buffer systems; solubilizers, for example beta-cyclodextrin, and if desired, conventional flavors or colorants. Oral formulations may also include or may be formulated as conventional formulations, such as tablets or granules.

Oral formulations may optionally further include taste-masking components to optimize the taste) perception of the oral formulation. Examples of such taste-masking components may be citrus-, licorice-, mint-, grape-, black currant- or eucalyptus-based flavorants known to those well-skilled in the art.

Within the scope of the present invention encompassed are also embodiments wherein the taste masking is achieved by incorporation into taste-masking particles, e.g. carrier particles as described herein.

The form of dosage for intranasal administration may include solutions, suspensions or emulsions of the active compound in a liquid carrier in the form of nose drops. Suitable liquid carriers include water, propylene glycol and other pharmaceutically acceptable alcohols. For administration in drop form formulations may suitably be put in a container provided e.g. with a conventional dropper/closure device, e.g. comprising a pipette or the like, preferably delivering a substantially fixed volume of composition/drop. The dosage forms may be sterilized, as required. The dosage forms may also contain adjuvants such as preservatives, stabilizers, emulsifiers or suspending agents, wetting agents, salts for varying the osmotic pressure or buffers, as required. Buffer systems may include for example phosphate buffer, carbonate buffer, citrate buffer, citrate-phosphate buffer and other pharmaceutically acceptable buffer systems. Intranasal formulations may optionally further include smell-masking components to optimize the smell.

For parenteral administration, liquid dosage units can be prepared containing the inventive composition and a sterile carrier, or the parts of the inventive kit of parts, and a sterile carrier. The parenteral solutions are normally prepared by dissolving the compound in a carrier and sterilizing by filtration, autoclavation, before filling suitable vials or ampoules and sealing.

Adjuvants, such as local anesthetics, preservatives and buffering agents can be added to the pharmaceutical composition or to the parts of the kit of parts of the present invention. In order to increase stability, the pharmaceutical composition or the parts of the kit of parts can be frozen after filling the vial and the water can be removed under vacuum. A surfactant or humectant can be advantageously included in the pharmaceutical composition or in the parts of the kit of parts in order to facilitate uniform distribution of the inventive composition or the parts of the inventive kit of parts.

Topical formulations include or are preferably ointments, creams, lotions, gels, gums, solutions, pastes or may contain liposomes, micelles or microspheres.

In a further embodiment, the present invention relates to the composition of the present invention, the salt of the present invention, the kit of parts of the present invention, or the pharmaceutical composition of the present invention for use as a medicament.

The medicament comprising the composition of the present invention, the salt of the present invention, the kit of parts of the present invention, or the pharmaceutical composition of the present invention can be used in the treatment of a number of diseases and disorders. The said diseases and disorders are preferably selected from the following:
a) treatment of depression, depressive episode, major depressive disorder, dysthymia, double depression, seasonal affective disorder, treatment-resistant depression, depressive episodes in bipolar disorder, postpartum depression, premenstrual dysphoric disorder, and/or stress-related affective disorders, e.g. burnout or depression in patients with chronic somatic disorders;
b) treatment of anxiety such as panic attacks, panic disorder, acute stress disorder, agoraphobia, generalized anxiety disorder, separation anxiety disorder, social phobia, specific phobia, substance-induced anxiety disorder; treatment of obsessive-compulsive disorder, treatment of post-traumatic stress disorder, treatment of attachment disorders; and/or treatment of attention deficit disorders, such as attention-deficit hyperactivity disorder (ADHD), autism and autism-spectrum disorders, and/or impulse control disorder;
c) treatment and prevention of substance-related and/or behavioral addictions (such as gambling, eating, digital media, exercise or shopping); treatment of substance addiction, drug dependence, tolerance, dependence or withdrawal from substances including alcohol, amphetamines, cannabis, cocaine, caffeine, stimulants, research chemicals, hallucinogens, inhalants, nicotine, opioids, GHB, dissociatives (including ketamine, phencyclidine), sedatives, hypnotics or anxiolytics; treatment of smoking addiction; and/or as an agent to aid quitting smoking,
d) as a support agent for psychotherapy and/or psychoanalysis;
e) as a diagnostic aid for dysfunctions, and/or mental and somatic disorders.
f) treatment of sexual dysfunction;
g) treatment of neuroses; and/or as an agent for inducing deep relaxation;
h) as an agent for pharmacological induction of meditative states;
i) treatment of tendency to aggressive behavior of the patient against himself and against other persons; and/or treatment of behavioral disorders and socially harmful behavior;
j) treatment of alexithymia; and/or improvement of mentalization and social skills (e.g. in attachment/developmental disorders, autism spectrum disorders);
k) stimulation of oxytocin release;
l) as agent for increasing the concentration of neurotransmitters in the central nervous system; as agent for increasing the concentration of serotonin in the central nervous system; and/or as agent for increasing the concentration of dopamine in the central nervous system;
m) as neuroprotective and neuroregenerative agent; treatment of movement disorders such as Parkinson's disease and essential tremor; sleep and autonomic nervous system disorders; Alzheimer's disease and other types of dementia; as an agent to support stroke rehabilitation through angiogenesis and a reduction of infarct volume and neuronal cell death; treatment of neuronal damage due to excessive substance abuse; as anti-aging agent, regenerative agent, prevention and treatment of signs of aging; protection from free radical damage; and/or protection and improvement of damage by ionizing radiation;
n) as appetite regulation agent; as weight loss agent; treatment and prevention of obesity; treatment of eating disorders including anorexia, bulimia, and binge eating disorder; activation of lipid metabolism and physiological fat burning; activation of carbohydrate metabolism; activation of physiological glycogen combustion; treatment and prevention of diabetes; and/or treatment of insulin resistance;
o) treatment of inflammation; treatment of chronic low grade inflammation, treatment of autoimmune disorders, including rheumatoid arthritis, systemic lupus erythematosus, inflammatory bowel disease, multiple sclerosis and other demyelinating diseases, type 1 diabetes mellitus, Guillain-Barre syndrome, and/or psoriasis; treatment of infectious diseases (preferably caused by fungi infection, helminth infection, or bacterial infection); treatment of ulcers, asthma, and bronchitis; and/or treatment of autoinflammatory diseases, including Crohn's disease, and/or Behcet's disease.
p) stimulation of immune response;
q) as an antineoplastic and antimetastatic agent; treatment and/or prevention of cancer, abnormal cell growth and mutation
r) treatment and/or prevention of cardiovascular diseases; treatment of abnormal blood pressure and abnormal heart rate
s) treatment of pain, psychosomatic pain, intestinal pain, perimenstrual pain, migraine and other types of headaches, neuropathic pain, phantom pain, musculoskeletal pain, rheumatic pain, and arthritis

It is to be understood that the above list of diseases is only given as specific examples and is not to be interpreted as limiting the present invention. Among the above, the preferred one(s) are one or more selected from a), b), and c).

In a further embodiment, the present invention relates to the composition of the present invention, the salt of the present invention, the kit of parts of the present invention or the pharmaceutical composition of the present invention for use in the treatment and/or prevention of a psychiatric, psychosomatic or somatic disorder.

Accordingly, the present invention relates as well to use of the composition of the present invention, the salt of the present invention, the kit of parts of the present invention or the pharmaceutical composition of the present invention for manufacture of a medicament for treatment and/or prevention of a psychiatric, psychosomatic or somatic disorder.

Further accordingly, the present invention relates to a method of treatment (and/or prevention) of a psychiatric, psychosomatic or somatic disorder, the method comprising the step of administering to the individual in need thereof of the composition of the present invention, the salt of the present invention, the kit of parts of the present invention or the pharmaceutical composition of the present invention. It is to be understood that the composition of the present invention, the salt of the present invention, the kit of parts of the present invention or the pharmaceutical composition of the present invention is to be administered in a therapeutically effective amount.

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

Preferably, within the scope of the present invention, the psychiatric, psychosomatic or somatic disorder is a psychiatric or neurodegenerative disorder. Preferably, said psychiatric disorder is selected from depression, stress-related affective disorder, major depressive disorder, dysthymia, treatment-resistant depression, burnout, anxiety, post-traumatic stress disorder, addiction, eating disorder, and obsessive-compulsive disorder. Preferably, said neurodegenerative disorder is selected from Parkinson's disease, essential tremor, stroke, multiple sclerosis and other demyelinating diseases, neuroinflammation, autonomic dysfunction, neuropathic or phantom pain, migraine and other types of headaches, neuronal damage due to excessive substance abuse, Alzheimer's disease and other types of dementia. Preferably, multiple sclerosis and other demyelinating diseases relates to multiple sclerosis. Preferably, Alzheimer's disease and other types of dementia relate to Alzheimer's disease.

Preferably, it is to be understood that said composition comprising harmine, or said salt of harmine is to be administered simultaneously, separately or sequentially with DMT or its pharmaceutically acceptable salt, as discussed hereinabove.

Preferably, within the scope of the present invention, the ratio of the dose of harmine to the dose of DMT is between 0.5 to 2.0. More preferably, said ratio is between 0.75 and 1.5. Even more preferably, said ratio is about 1.0.

Within the scope of the present invention, harmine, as comprised in the composition of the present invention, salt of the present invention, pharmaceutical composition of the present invention, or kit of parts of the present invention, and DMT, (optionally as comprised within the pharmaceutical composition of the present invention or the kit of parts of the present invention) can be administered to a subject as a single bolus dose. Preferably, within said single bolus dose, the total dose of harmine is between 5 mg and 200 mg (in case a salt or solvate of harmine is administered, the amount is to be recalculated to account for the mg content of harmine in said salt) and/or the total dose of DMT is between 5 mg and 100 mg (in case a salt or solvate is used, the amount is to be recalculated to account for the mg content of DMT in said salt).

Further within the scope of the present invention, harmine, as comprised in the composition of the present invention, salt of the present invention, pharmaceutical composition of the present invention, or kit of parts of the present invention, and DMT, (optionally as comprised within the pharmaceutical composition of the present invention or the kit of parts of the present invention) can be administered to a subject incrementally. It is preferred that each increment of harmine is between 5 mg and 80 mg, and/or each increment of DMT is between 5 mg and 50 mg. It is to be understood that harmine and DMT may be administered together, separately, or sequentially. Furthermore, it is preferred that the total dose of harmine is between 100 mg and 300 mg and/or the total dose of DMT between 50 mg and 150 mg. It is further preferred that the interval between the increments is between 5 and 60 minutes, preferably between 15 and 60 minutes.

It is further disclosed herein that beyond combinations with DMT, harmine can also be used as an individual agent in the treatment of a number of diseases and/or disorders.

Accordingly, the composition comprising harmine of the present invention, the pharmaceutical composition comprising harmine or a pharmaceutically acceptable salt thereof of the present invention, a salt of harmine of the present invention can be used for the treatment or prevention of a number of diseases and disorders. Preferably, said diseases and disorders are selected from Parkinson's disease, Alzheimer's disease and other types of dementias, stroke, multiple sclerosis, neurodegeneration/-inflammation, neuronal damage due to excessive substance abuse, autonomic dysfunction, pain syndromes, cardiovascular disorders, cancer, infectious diseases (preferably caused by fungi infection, helminth infection, or bacterial infection), diabetes, autoimmune disease, asthma, bronchitis, and arthritis.

The term about, as used herein, when used in the context of a numerical value, preferably refers to that value±10% of said value, more preferably to that value±5% of said value, even more preferably to that value±1% of said value, even more preferably to said value.

The invention will be illustrated in the following examples, which however are not to be construed as limiting.

### Examples

Testing the Physicochemical Properties of Different HRM Solid State Forms and their Suitability for the Development of Sublingual/Oromucosal Dosage Forms.

In Part 1 of this section, the physicochemical properties of several salts, polymorphs and composition of harmine were investigated. The main aim was to identify solid state forms or compositions of harmine with high solubility (>20% (w/v) in water, which is a physicochemical prerequisite, when delivering high drug loads (>50 mg) over the oromucosal route of administration. This is because all oromucosal dosage forms such as sublingual/buccal sprays, drops, films or melting tablets, require a high solubility of the compound, in order to ensure an acceptable size (e.g. drops/sprays: not more than 0.2 ml/dose; melting tablets: not more than 0.5 ml/dose; preferably not more than 0.1 ml/dose) and thus palatability of the pharmaceutical product. Likewise, the loading of carrier particles, e.g. TIP particles requires high solubility of the compound in water, EtOH or DMSO in order to yield a scalable process and high loading efficiency.

In Part 2 of this section, several pharmaceutical formulations of harmine were developed. Thereby, the oromucosal absorption, pharmacokinetics, and palatability of the formulations were assessed. It was the aim of this study to identify a sublingual formulation that is palatable (not bitter, no bad taste), shows high bioavailability, reduced first-pass metabolism and low intersubject variability.

Part 3 outlines a planned study aiming at further investigating in vivo properties (PKPD) of the most promising candidate identified in Part 2.

### PART 1: Physicochemical Screening (Solid State) of Different Harmine Salts, Polymorphs and Compositions.

To identifiy new solid state forms of harmine, following experiments were conducted:
1) Polymorphscreenings of Harmine HCl
2) Harmine Salt screenings
3) Screening compositions containing harmine and different acids

### Polymorphscreening Harmine HCl—Summary:

The drug substance Harmine-HCl (was investigated for polymorphism and pseudopolymorphism by thermal analysis (DSC, TGA), spectroscopy (IR, Raman, NMR), powder X-ray diffraction (XRPD) and crystallization experiments from various solvents. Solubility experiments were also conducted. The provided Harmine-HCl was measured with a so called “Chip-DSC” in order to protect the DSC sensor from HCl emitting gas. According to the CAS (Chemical Abstract Service), the melting point (decomposition) is 272-275° C., whereas the Chip-DSC shows direct decomposition from approx. 260° C. (20 K/min) without a clear melting peak. Thermogravimetry at 10 K/min before decomposition showed a not very pronounced two-stage mass decrease. Up to 200° C. the mass decreases by 4.2% and between 200° C. and 300° C. the mass decreases by 12.5% and after 300° C. by further 60%.

Harmine-HCl converts to the form scNF1 after static storage (RT/100% RH for 7 days). The form scNF1 was also created in the course of cooling crystallizations in water/acetone (70/30), (70/30), water/dioxane water/THF (70/30) and water/acetonitrile (70/30). In addition, the form scNF1 was created by gas diffusion crystallizations of water/1,4-dioxane, water/2-propanol and water/acetonitrile. In long-term slurries, scNF1 was successfully produced with acetone/water (80/20) and acetonitrile/water (90/10). Rapid precipitations of the form scNF1 with the antisolvent water are achieved with the solvents acetone and acetonitrile. Form scNF1 can also be crystallized by evaporating the solvent water.

The form scNF1 is Harmine-HCl dihydrate, as could be determined by means of TGA after static storage (RT/100% RH for 7 days). After 5 weeks at ambient conditions, traces of Harmine-HCl were identified in the powder diffractogram, next to the dihydrate form. A more detailed characterization of the form scNF1 was not requested by the client.

In total, 162 experiments were performed during the polymorphism screening. Apart from the dihydrate, no further new solid forms of Harmine-HCl were obtained.

### Harmine Salt Screening—Summary

The drug substance Harmine was investigated for a salt screening of different salts by crystallization experiments from various solvents and grinding experiments. Solubility experiments were also conducted before starting the salt screening.

In the course of the screening performed, 14 new solid-phases were detected by using XRPD. The new solid-state entities are listed in the following table 1 below:

TABLE 1
New solid state forms of harmine

| SOLID- | | |
|---|---|---|
| CHEM | Salt-Former | Evaluation/Remarks |
| SC-NF1 | Succinic acid | Probably 1:1 salt with succinic acid. In the ¹H-NMR |
| | | spectrum slightly less succinic acid than API, but |
| | | according to XRPD API was present in excess. TGA |
| | | and dry grinding suggest that a hydrate may be present. |
| | | DSC investigations show that the thermal behavior is |
| | | insufficiently. |
| SC-NF2 | L(+)-Tartaric | According to the ¹H-NMR spectrum 1:1 salt with L- |
| | acid | tartaric acid. Possibly a solvate/hydrate. DSC |
| | | investigations show that the thermal behavior is |
| | | insufficiently. |
| SC-NF3 | L- Ascorbic | According to the ¹H-NMR spectrum 1:1 salt with L- |
| | acid | ascorbic acid. Possibly a solvate/hydrate. DSC |
| | | investigations show that the thermal behavior is |
| | | insufficiently. |
| SC-NF4 | L(−)-Malic | According to the ¹H-NMR spectrum 1:1 salt with L- |
| | acid | malic acid. DSC investigations show that the thermal |
| | | behavior is insufficiently. |
| SC-NF5 | Citric acid | According to the ¹H-NMR spectrum 1:1 salt with |
| | | citric acid. DSC investigations show that the |
| | | thermal behavior is insufficiently. |
| SC-NF6 | L(+)-Tartaric | According to the ¹H-NMR spectrum 1:1 salt with |
| | acid | tartaric acid. DSC investigations show that the |
| | | thermal behavior is insufficiently. |
| SC-NF7 | L(−)-Malic | According to the ¹H-NMR spectrum 1:1 salt with L- |
| accompanying | acid | malic acid. DSC investigations show that the thermal |
| SC-NF4 | | behavior is insufficiently. |
| SC-NF8 | Methanesulfonic | According to the ¹H-NMR spectrum 1:1 salt with |
| | acid | methanesulfonic acid. DSC investigations show that |
| | | the thermal behavior is insufficiently. |
| SC-NF9 | Sulfuric acid | According to the ¹H-NMR spectrum 1:1 salt with sulfuric |
| | | acid. DSC investigations show that the thermal behavior |
| | | is insufficiently. |
| SC-NF10 | L- Tartaric | New form apparently unstable since a repeat of the |
| | acid | XRPD analysis reveals that a physical mixture of |
| | | harmine + tartaric acid occurred. |
| SC-NF11 | L- Ascorbic | New form apparently unstable since a repeat of the |
| | acid | XRPD analysis reveals that a physical mixture of |
| | | harmine + ascorbic acid occurred. |
| SC-NF12 | Phosphoric | Since the new form does not show sufficient solubility |
| | acid | in any deuterated solvent, an IR spectroscopic study |
| | | was performed. These investigations show that the 1:1 |
| | | salt of phosphoric acid has been formed. The DSC |
| | | thermogram reveals a very acceptable thermal behavior. |
| SC-NF13 | L- Tartaric | According to the XRPD diffractogram a partially |
| | acid | crystalline new solid-state form could be obtained. |
| SC-NF14 | L- Ascorbic | According to the XRPD diffractogram a very |
| | acid | crystalline new solid-state form could be obtained. |

### Experimental Set Up

In the following table 2, the acids used in the salt screening are summarized:

TABLE 2
Acids used in the salt screening

| | | GRAS | | | | Ph. Eur. | |
|---|---|---|---|---|---|---|---|
| | | safety | | | | statement of | available data |
| # | Name | level | MW | pka (½) | ΔpKa | solubility | solubilty (water) |
| 0 | Harmine | — | 212.25 | 7.45 | — | — | — |
| 1 | Succinic acid | 1 | 118.09 | 4.16/5.61 | 3.29/1.84 | soluble | 58 g/L 20° C. |
| 2 | L(+)-Tartaric acid | 1 | 150.09 | 3.02/4.36 | 4.43/3.09 | very easily | 1394 g/L 20° C. |
| | | | | | | souble | |
| 3 | L-Ascorbic acid | 1 | 176.13 | 4.17 | 3.28 | easily souble | 333 g/L 20° C. |
| 4 | L(−)-Malic acid | 1 | 134.09 | 3.459/5.097 | 3.99/2.35 | easily souble | 363.5 g/L 20° C. |
| 5 | Citric acid | 1 | 192.13 | 3.128/4.761 | 4.32/2.69 | very easily | 1450 g/L 20° C. |
| | | | | | | souble | |
| 6 | Methanesulfonic | 1 | 96.11 | −1.2 | 8.65 | — | completely |
| | acid | | | | | | miscible |
| 7 | Phosphoric acid | 1 | 98.00 | 1.96/7.12/12.32 | 5.49/0.33/4.87 | — | completely |
| | | | | | | | miscible |
| 8 | Sulfuric acid | 1 | 98.08 | −3/1.92 | 10.45/5.53 | — | completely |
| | | | | | | | miscible |

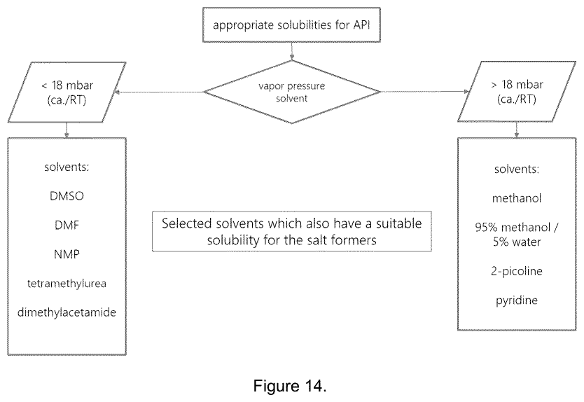

In the following table 3, the different solvents used in the salt screening are summarized. FIG. 14 shows a schematic depiction for the choice of solvents.

TABLE 3
Information about the used solvents

| | | Anti-Solvent |
|---|---|---|
| # | Solvent | practically insoluble |
| 1 | dimethyl sulfoxid (DMSO) | dimethyl carbonate |
| | almost resolved - 13 mg/mL | |
| 2 | pyridine | tetrahydrofurane (THF) |
| | easily souble - 205 mg/mL | (25° C. \| 45° C.) |
| 3 | dimethylformamid (DMF) | acetonitrile |
| | souble - 33 mg/mL | (25° C. \| 45° C.) |
| 4 | 1-methyl-2-pyrrolidone (NMP) | methanol |
| | souble - 80 mg/mL | (25° C. \| 45° C.) |
| 5 | 2-picoline | acetone |
| | almost resolved - 10 mg/mL | (25° C. \| 45° C.) |
| 6 | tetramethylurea | 2-butanone (MEK) |
| | souble - 40 mg/mL | (25° C. \| 45° C.) |
| 7 | dimethylacetamide | dichloromethane |
| | souble - 40 mg/mL | (25° C. \| 45° C.) |
| 8 | methanol (45° C.) | chloroform |
| | almost resolved - 12 mg/mL | |
| 9 | 95% methanol \| 5% water | 1,4-dioxane |
| | almost resolved | |
| 10 | | water |
| 11 | | veratrol |
| 12 | | p-xylene |
| 13 | | sulfolane |
| 14 | | benzonitrile |
| 15 | | 95% acetonitrile \| |
| | | 5% water (45° C.) |
| 16 | | 95% acetone \| |
| | | 5% water (45° C.) |

A summary of the experiments carried out API & salt former in a molar ratio 1:1 is shown in the table 4 below:

TABLE 4
Summary of the experiments carried out API & salt former in a molar ratio 1:1.

| # | SOLID-CHEM | Saltformer | Solvents | Screening-method | XRPD |
|---|---|---|---|---|---|
| 1 | ethHAR001EVA013 | succinic acid | methanol | evaporation - 45° C. | scNF1 |
| 2 | ethHAR001EVA014 | L(+)-tartaric acid | methanol | evaporation - 45° C. | scNF6 |
| 3 | ethHAR001EVA015 | L-ascorbic acid | methanol | evaporation - 45° C. | amorph |
| 4 | ethHAR001EVA016 | L(−)-malic acid | methanol | evaporation - 45° C. | scNF4 & scNF7 |
| 5 | ethHAR001EVA017 | citric acid | methanol | evaporation - 45° C. | scNF5 |
| 6 | ethHAR001EVA018 | succinic acid | pyridine | evaporation - 25° C. | scNF1 & scNF0 |
| 7 | ethHAR001EVA019 | L(+)-tartaric acid | pyridine | evaporation - 25° C. | scNF13 |
| 8 | ethHAR001EVA020 | L-ascorbic acid | pyridine | evaporation - 25° C. | scNF14 |
| 9 | ethHAR001EVA021 | L(−)-malic acid | pyridine | evaporation - 25° C. | scNF4 |
| 10 | ethHAR001EVA022 | citric acid | pyridine | evaporation - 25° C. | scNFS |
| 11 | ethHAR001EXP113 | methanesulfonic acid | water | evaporation - 45° C. | scNF8 |
| 12 | ethHAR001EXP114 | sulfuric acid | water | evaporation - 45° C. | scNF9 |
| 13 | ethHAR001EXP115 | phosphoric acid | Water | evaporation - 45° C. | scNF12 |
| 14 | entHAR001GRI001 | succinic acid | water/MeOH 1:1 | grinding - 30 min | scNF1 & API |
| 15 | entHAR001GRI002 | L(+)-tartaric acid | water/MeOH 1:1 | grinding - 30 min | scNF2 |
| 16 | entHAR001GRI003 | L-ascorbic acid | water/MeOH 1:1 | grinding - 30 min | scNF3 |
| 17 | entHAR001GRI004 | L(−)-malic acid | water/MeOH 1:1 | grinding - 30 min | scNF4 & traces |
| | | | | | of API |
| 18 | entHAR001GRI005 | citric acid | water/MeOH 1:1 | grinding - 30 min | scNF5 |
| 19 | entHAR001GRI013 | succinic acid | dry | grinding - 30 min | scNF1 & API |
| 20 | entHAR001GRI014 | L(+)-tartaric acid | dry | grinding - 30 min | D2: scNF10; |
| | | | | | D8: API & CoF |
| 21 | entHAR001GRI015 | L-ascorbic acid | dry | grinding - 30 min | D2: scNF11 & API; |
| | | | | | D8: API & CoF |
| 22 | entHAR001GRI016 | L(−)-malic acid | dry | grinding - 30 min | scNF4 |
| | | | | | (low |
| | | | | | crystallinity) |
| 23 | entHAR001GRI017 | citric acid | dry | grinding - 30 min | scNFS |

### Experimental Procedure for Each Harmine

scNF1—Salt with Succinic Acid—Exemplary ethHAR001EVA013:

35.6 mg of the active ingredient as well as 19.8 mg of the salt-former are weighed into a 4 mL vial. 4 mL methanol are added at 45° C., the vessel is tightly closed and stirred for ten minutes on a magnetic stir plate. A syringe, a cannula and a 0.2 μm ReZist® syringe filter attachment are stored in a drying cabinet preheated to 47° C. The solution is quickly drawn up completely with a syringe and filtered through the 0.2 μm ReZist® filter into a new 4 mL vial at 45° C. for the evaporation experiment. After the end of the evaporation experiment, the sample is sealed. In the following table 5, further experiments to obtain harmine succinate salts:

TABLE 5
Experiments to obtain harmine succinate salts
Further experiments to obtain succinic acid: API salt

| | | |
|---|---|---|
| scNF1 | succinic acid | ethHAR001EVA013 = methanol, |
| | | evaporation at 45° C. |
| | | ethHAR001EVA018 = pyridine, |
| | | evaporation at rt |
| | | ethHAR001GRI001 = 30 min |
| | | grinding with two drops water/ |
| | | MeOH 1:1 |
| | | ethHAR001GRI013 = 30 min |
| | | grinding (dry) |

scNF2—Salt with L(+)-tartaric acid—ethHAR001GRI002:

40 mg of the API as well as 28.3 mg of the salt-former L(+)-tartaric acid are weighed into a grinding jar filled with ten ceramic balls (3×5 mm, 7×2 mm). Two drops of water/methanol were added and the vessel is tightly closed. The grinding jar is inserted into the Fritsch Micromill Pulverisette 7 and the sample is ground for 30 minutes. The following conditions are selected for grinding:
ten ceramic balls (3×5 mm, 7×2 mm)
Temperature: fume cupboard environment-room temperature
Grinding time: 30 minutes

The solid residues are prepared in a 4 mL vial with PTFE.

scNF3—Salt with L-Ascorbic Acid—ethHAR001GRI003:

40 mg of the API as well as 33.2 mg of the salt-former L-ascorbic acid are weighed into a grinding jar filled with ten ceramic balls (3×5 mm, 7×2 mm). Two drops of water/methanol were added and the vessel is tightly closed. The grinding jar is inserted into the Fritsch Micromill Pulverisette 7 and the sample is ground for 30 minutes. The following conditions are selected for grinding:
ten ceramic balls (3×5 mm, 7×2 mm)
Temperature: fume cupboard environment-room temperature
Grinding time: 30 minutes

The solid residues are prepared in a 4 mL vial with PTFE.

scNF4—Salt with L(−)-malic acid—exemplary—ethHAR001EVA016:

40 mg of the active ingredient as well as 25.3 mg of the salt-former L-ascorbic acid are weighed into a 4 mL vial. 4 mL pyridine are added at 25° C., the vessel is tightly closed and stirred for ten minutes on a magnetic stir plate. A syringe, a cannula and a 0.2 μm ReZist® syringe filter attachment are stored in a drying cabinet preheated to 25° C. The solution is quickly drawn up completely with a syringe and filtered through the 0.2 μm ReZist® filter into a new 4 mL vial at 25° C. for the evaporation experiment. After the end of the evaporation experiment, the sample is sealed. In the following table 6, further experiments to obtain harmine malic acid salts:

TABLE 6
Experiments to obtain harmine malic acid salts
Further experiments to obtain L(−)-malic acid: API salt

| | | |
|---|---|---|
| scNF4 | L(−)-malic acid | ethHAR001GRI004 = 30 min |
| | | grinding with two drops water/ |
| | | MeOH 1:1 |
| | | ethHAR001GRI016 = 30 min |
| | | grinding (dry) |
| | | ethHAR001EVA021 = pyridine, |
| | | evaporation at rt |

scNF5—Salt with Citric Acid—Exemplary—ethHAR001EVA017:

35.6 mg of the active ingredient as well as 32.2 mg of the salt-former citric acid are weighed into a 4 mL vial. 4 mL methanol are added at 45° C., the vessel is tightly closed and stirred for ten minutes on a magnetic stir plate. A syringe, a cannula and a 0.2 μm ReZist® syringe filter attachment are stored in a drying cabinet preheated to 47° C. The solution is quickly drawn up completely with a syringe and filtered through the 0.2 μm ReZist® filter into a new 4 mL vial at 45° C. for the evaporation experiment. After the end of the evaporation experiment, the sample is sealed. In the following table 7, further experiments to obtain harmine citric acid salts:

TABLE 7
Experiments to obtain harmine citric acid salts
Further experiments to obtain citric acid: API salt

| | | |
|---|---|---|
| scNF5 | citric acid | ethHAR001GRI005 = 30 min grinding with |
| | | two drops water/MeOH 1:1 |
| | | ethHAR001GRI017 = 30 min grinding (dry) |
| | | ethHAR001EVA017 = methanol, evaporation |
| | | at 45° C. |
| | | ethHAR001EVA022 = pyridine, evaporation |
| | | at rt |

scNF6—Salt with L(+)-Tartaric Acid—ethHAR001EVA014:

35.6 mg of the active ingredient as well as 25.1 mg of the salt-former L(+)-tartaric acid are weighed into a 4 mL vial. 4 mL methanol are added at 45° C., the vessel is tightly closed and stirred for ten minutes on a magnetic stir plate. A syringe, a cannula and a 0.2 μm ReZist® syringe filter attachment are stored in a drying cabinet preheated to 47° C. The solution is quickly drawn up completely with a syringe and filtered through the 0.2 μm ReZist® filter into a new 4 mL vial at 45° C. for the evaporation experiment. After the end of the evaporation experiment, the sample is sealed.

cNF7 & scNF4—Salt with L(−)-malic acid—ethHAR001EVA016:

35.6 mg of the active ingredient as well as 22.5 mg of the salt-former L(−)-malic acid are weighed into a 4 mL vial. 4 mL methanol are added at 45° C., the vessel is tightly closed and stirred for ten minutes on a magnetic stir plate. A syringe, a cannula and a 0.2 μm ReZist® syringe filter attachment are stored in a drying cabinet preheated to 47° C. The solution is quickly drawn up completely with a syringe and filtered through the 0.2 μm ReZist® filter into a new 4 mL vial at 45° C. for the evaporation experiment. After the end of the evaporation experiment, the sample is sealed.

scNF8—Salt with Methanesulfonic Acid—ethHAR001EXP113:

40 mg of the active ingredient are weighed into a 4 mL vial. 1 mL water is added at 25° C., the vessel is tightly closed and stirred for ten minutes on a magnetic stir plate. The methanesulfonic acid (12 μL) is quickly added to the solution. The vial is sealed properly with a screw cap and additional application of Parafilm. The vial is placed on a magnetic stirrer by using following parameters:
stirring speed: 1000 rpm
temperature: fume hood environment—RT
stirring time: 5 days

The suspension is checked on a daily basis in order to verify a homogeneous suspension. After five days the slurry is filtered over a 13 mm Hirsch-funnel equipped with a Whatman® paper filter No 540. The solid residues are dried in the air (approx. one hour) and then placed in a new, clean 4 mL vial with a PTFE seal.

scNF9—Salt with Sulfuric Acid—ethHAR001EXP114:

40 mg of the active ingredient are weighed into a 4 mL vial. 1 mL water is added at 25° C., the vessel is tightly closed and stirred for ten minutes on a magnetic stir plate. The sulfuric acid (10.3 μL) is quickly added to the solution. A syringe, a cannula and a 0.2 μm ReZist® syringe filter attachment are stored in a drying cabinet preheated to 25° C. The solution is quickly drawn up completely with a syringe and filtered through the 0.2 μm ReZist® filter into a new 4 mL vial at 25° C. for the evaporation experiment. After the end of the evaporation experiment, the sample is sealed.

scNF10—Salt with L-Tartaric Acid—ethHAR001GRI014:

40 mg of the API as well as 28.3 mg of the salt-former L-tartaric acid are weighed into a grinding jar filled with ten ceramic balls (3×5 mm, 7×2 mm). The vessel is tightly closed. The grinding jar is inserted into the Fritsch Micromill Pulverisette 7 and the sample is ground for 30 minutes. The following conditions are selected for grinding:
ten ceramic balls (3×5 mm, 7×2 mm)
Temperature: fume cupboard environment-room temperature
Grinding time: 30 minutes

The solid residues are prepared in a 4 mL vial with PTFE.

scNF11—Salt with L-Ascorbic Acid—ethHAR001GRI015:

40 mg of the API as well as 28.3 mg of the salt-former L-ascorbic acid are weighed into a grinding jar filled with ten ceramic balls (3×5 mm, 7×2 mm). The vessel is tightly closed. The grinding jar is inserted into the Fritsch Micromill Pulverisette 7 and the sample is ground for 30 minutes. The following conditions are selected for grinding:
ten ceramic balls (3×5 mm, 7×2 mm)
Temperature: fume cupboard environment-room temperature
Grinding time: 30 minutes

The solid residues are prepared in a 4 mL vial with PTFE.

scNF12—Salt with Phosphoric Acid—ethHAR001EXP115:

40 mg of the active ingredient are weighed into a 4 mL vial. 1 mL water is added at 25° C., the vessel is tightly closed and stirred for ten minutes on a magnetic stir plate. The phosphoric acid (9.8 μL) is quickly added to the solution. The vial is sealed properly with a screw cap and additional application of Parafilm. The vial is placed on a magnetic stirrer by using following parameters:
stirring speed: 1000 rpm
temperature: fume hood environment—RT
stirring time: 5 days

The suspension is checked on a daily basis in order to verify a homogeneous suspension. After five days the slurry is filtered over a 13 mm Hirsch-funnel equipped with a Whatman® paper filter No 540. The solid residues are dried in the air (approx. one hour) and then placed in a new, clean 4 mL vial with a PTFE seal.

scNF13—Salt with L-Tartaric Acid—ethHAR001EVA019:

40 mg of the active ingredient as well as 28.3 mg of the salt-former L-tartaric acid are weighed into a 4 mL vial. 4 mL pyridine are added at 25° C., the vessel is tightly closed and stirred for ten minutes on a magnetic stir plate. A syringe, a cannula and a 0.2 μm ReZist® syringe filter attachment are stored in a drying cabinet preheated to 25° C. The solution is quickly drawn up completely with a syringe and filtered through the 0.2 μm ReZist® filter into a new 4 mL vial at 25° C. for the evaporation experiment. After the end of the evaporation experiment, the sample is sealed.

scNF14—Salt with L-Ascorbic Acid—ethHAR001EVA020:

40 mg of the active ingredient as well as 33.2 mg of the salt-former L-ascorbic acid are weighed into a 4 mL vial. 4 mL pyridine are added at 25° C., the vessel is tightly closed and stirred for ten minutes on a magnetic stir plate. A syringe, a cannula and a 0.2 μm ReZist® syringe filter attachment are stored in a drying cabinet preheated to 25° C. The solution is quickly drawn up completely with a syringe and filtered through the 0.2 μm ReZist® filter into a new 4 mL vial at 25° C. for the evaporation experiment. After the end of the evaporation experiment, the sample is sealed.

### Characterization of New Salt Forms

### Qualitative Solubility (Temperature-Dependent)

Four 250 mL glass jacketed beakers are set up, which are connected with silicone hoses and connected to a Julabo F25 HE thermostat in order to be able to set a constant temperature. In the beakers there is a Pt100 sensor for temperature monitoring, a stopper with a hole that is sealed with play dough and a vial holder with space for four 4 ml vials for a quadruple determination of the solubility. The beakers are located on a strong magnetic stirrer plate so that the temperature is distributed as homogeneously as possible in the liquid medium. Water is used as the medium. The Pt100 sensors and the Julabo are connected to a measuring computer, which controls the Julabo and the temperatures of the Pt100 sensors can be read.

A Sartorius LE26P microbalance with an accuracy of ±0.01 mg is used for the gravimetric determination of the solubility in mass percent.

4 ml vials are filled with 1-2 mL of the respective solvent and equipped with a stirring magnet. The solid to be measured is then added until it is supersaturated. The prepared vials are sealed and placed in the vial holder and equilibrated in the apparatus for 48 hours at the temperature to be examined. After 24 hours, an equilibrium between the solid and liquid phases should have been reached. The liquid phase is removed and transferred to a second vial via a Whatman 20 μm PTFE pre-syringe filter. The tools used, such as the pre-syringe filter, syringe and cannula, are initially cooled or heated to the temperature to be measured. The second vial is first empty, then weighed with the solution and finally evaporated again to ensure constant mass. The solubility in mass percent can then be calculated from this data.

### X-Ray Diffractometry

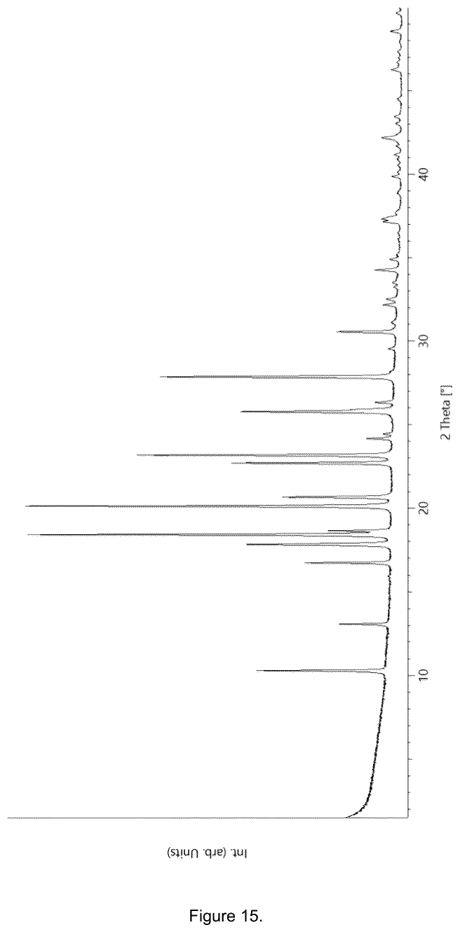

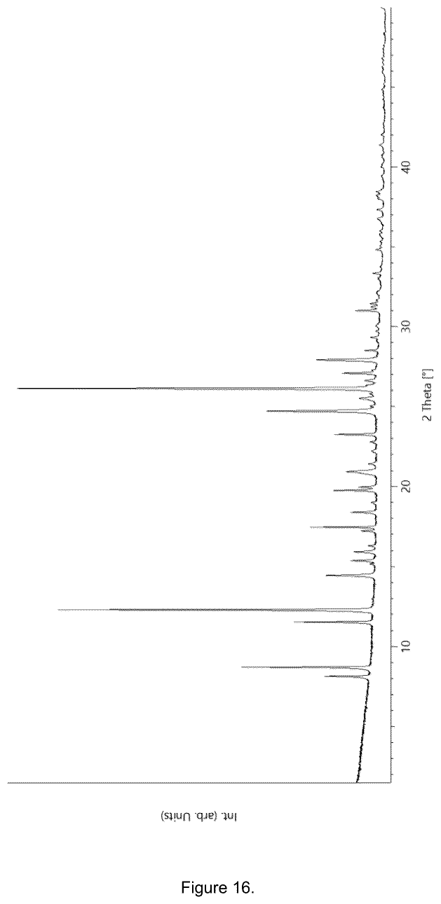

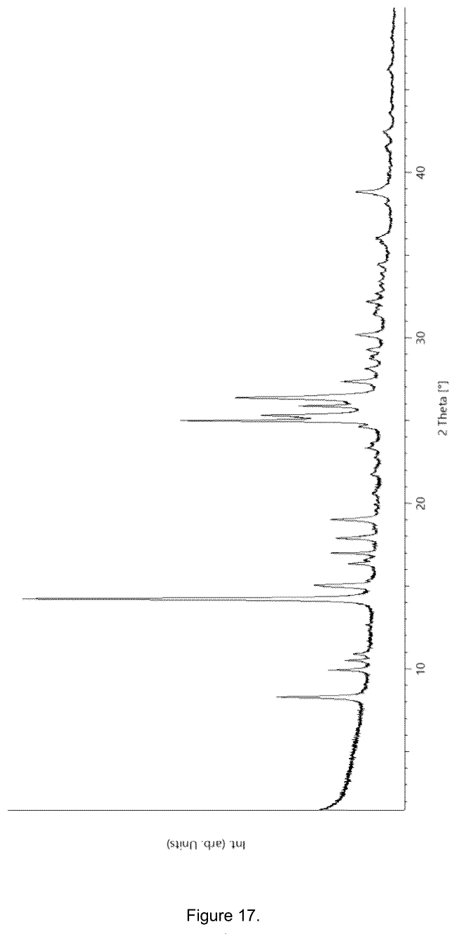

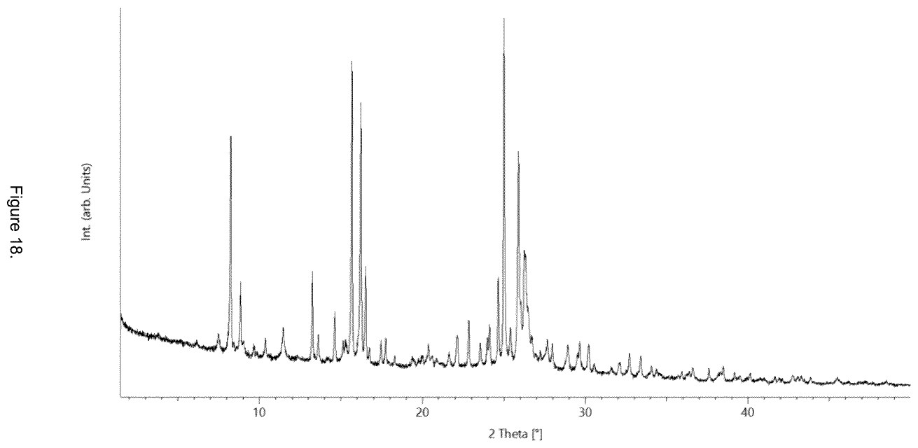

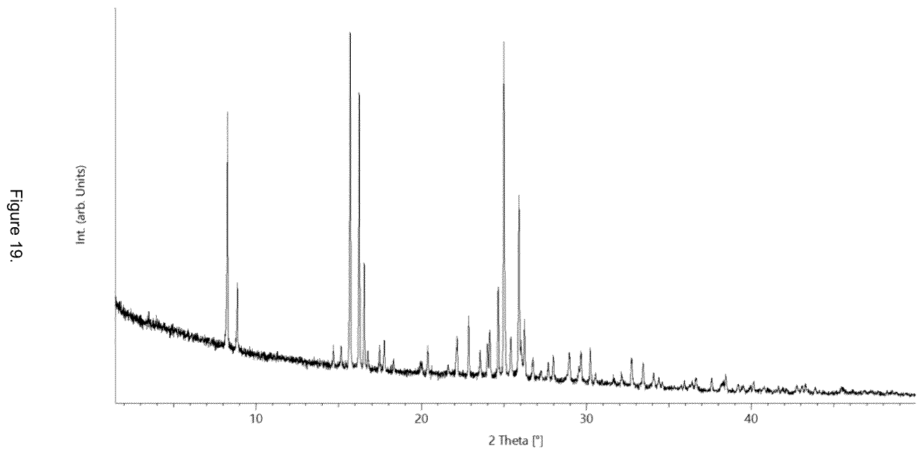

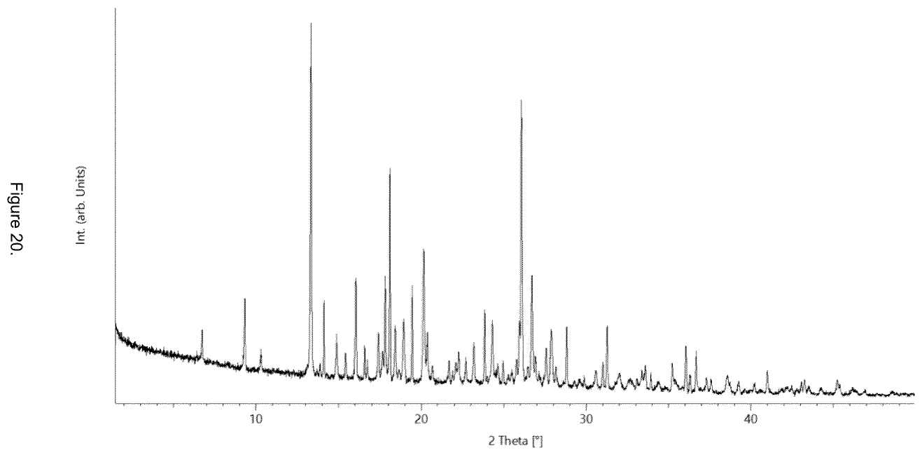

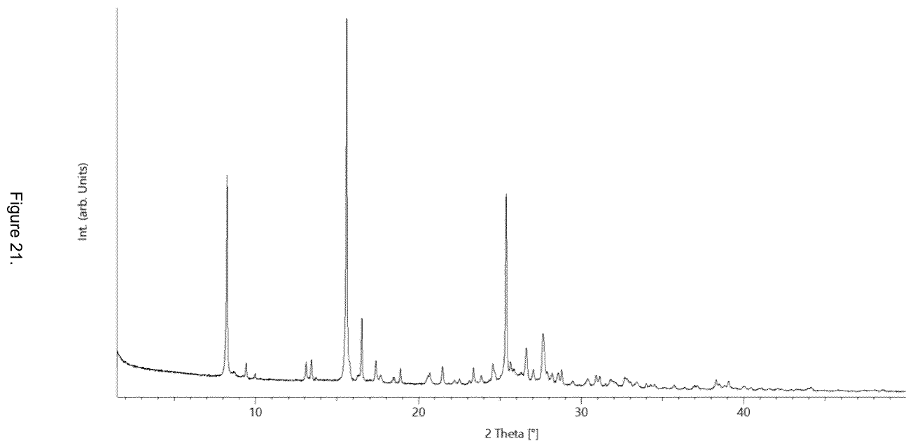

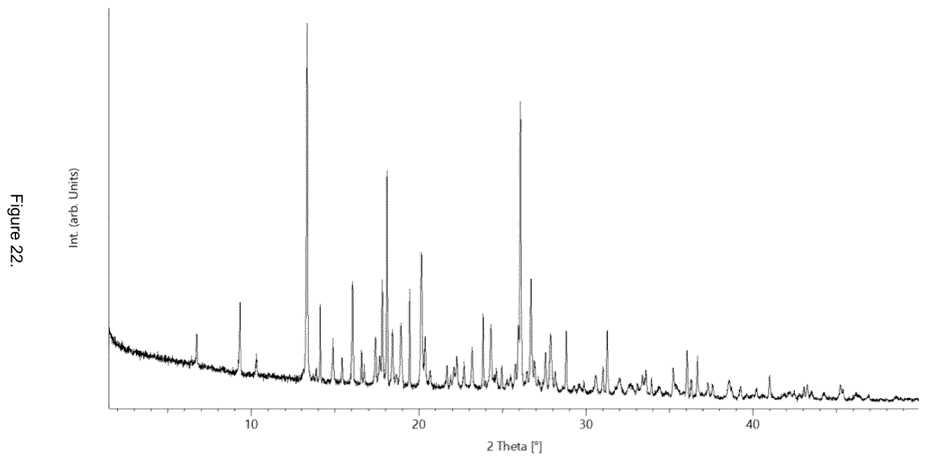

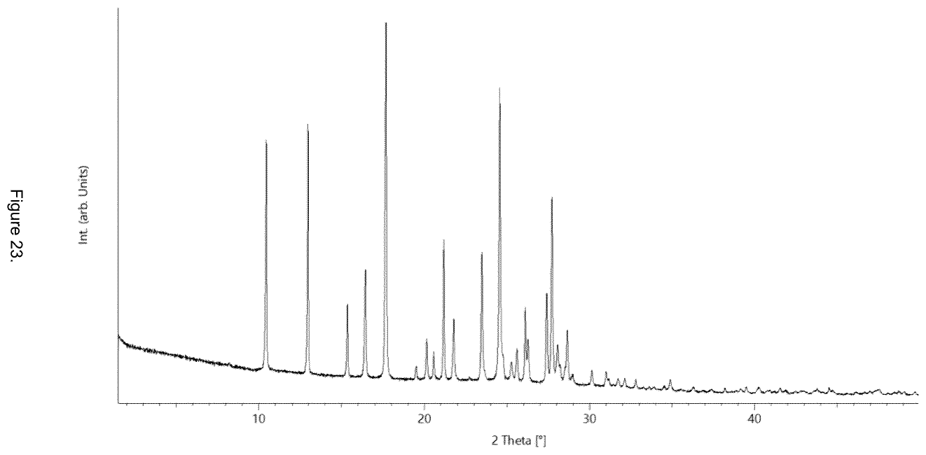

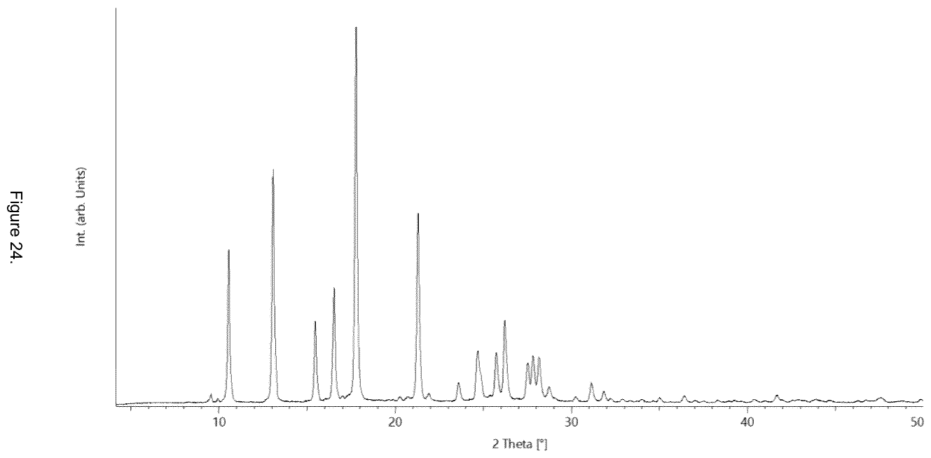

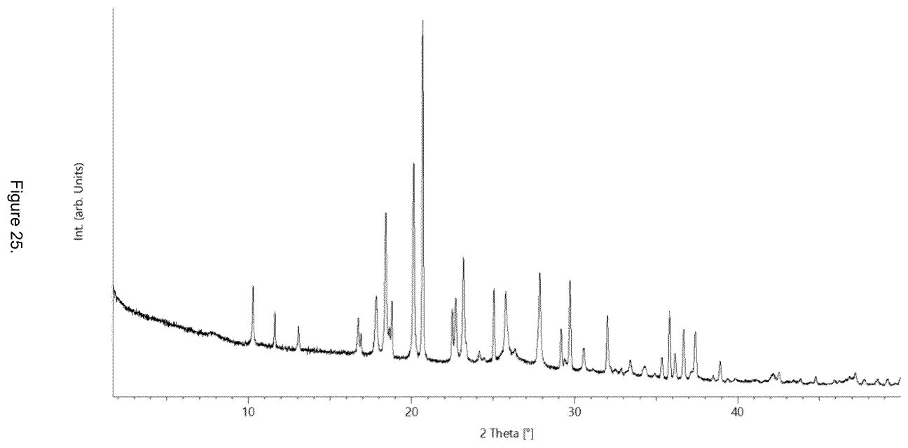

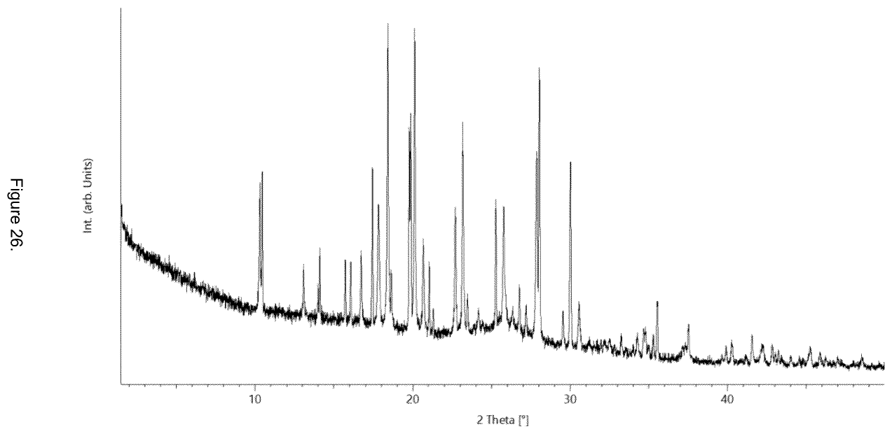

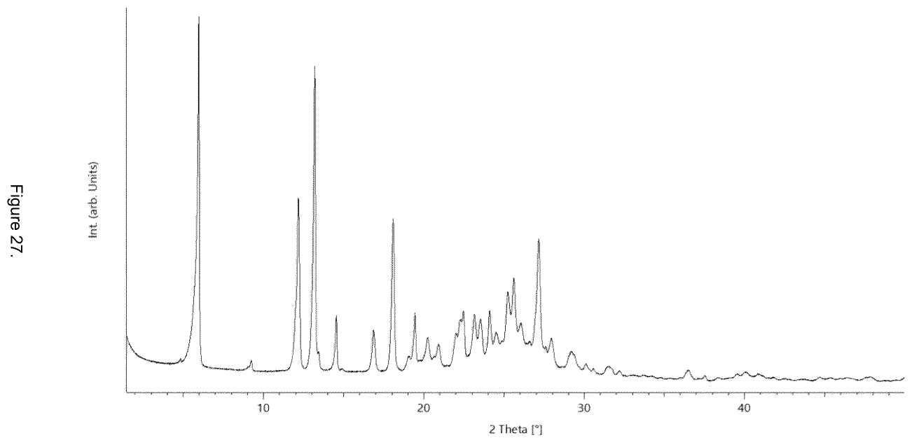

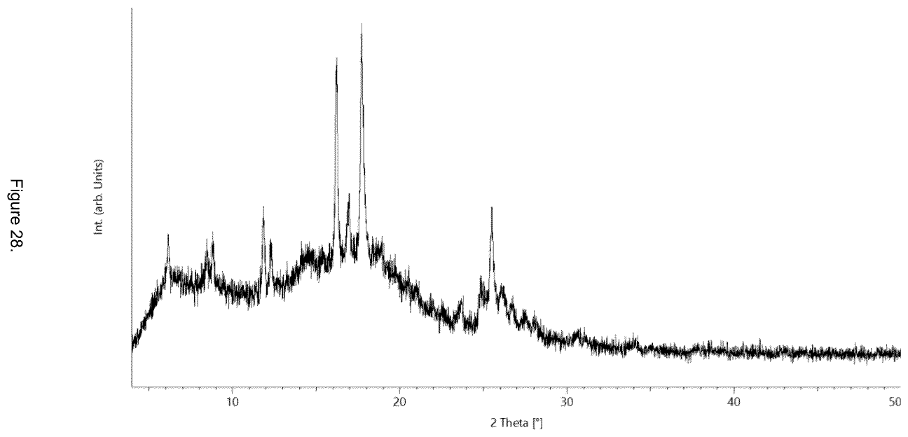

During the salt screening process, 14 new solid entities were found. The X-ray diffraction patterns including the X-ray diffraction pattern of Harmine are shown in FIGS. 15-29.

Differential Scanning calorimetry (DSC) and Thermogravimetry (TGA)

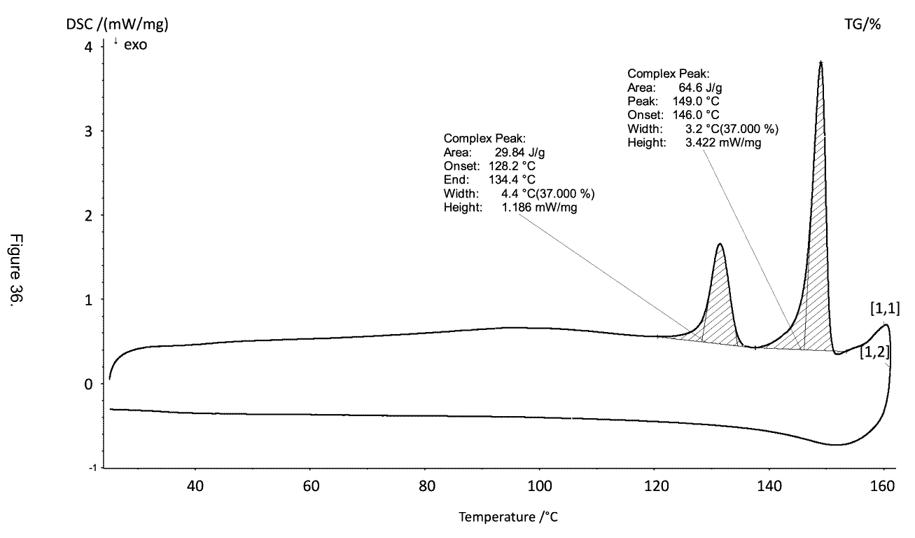

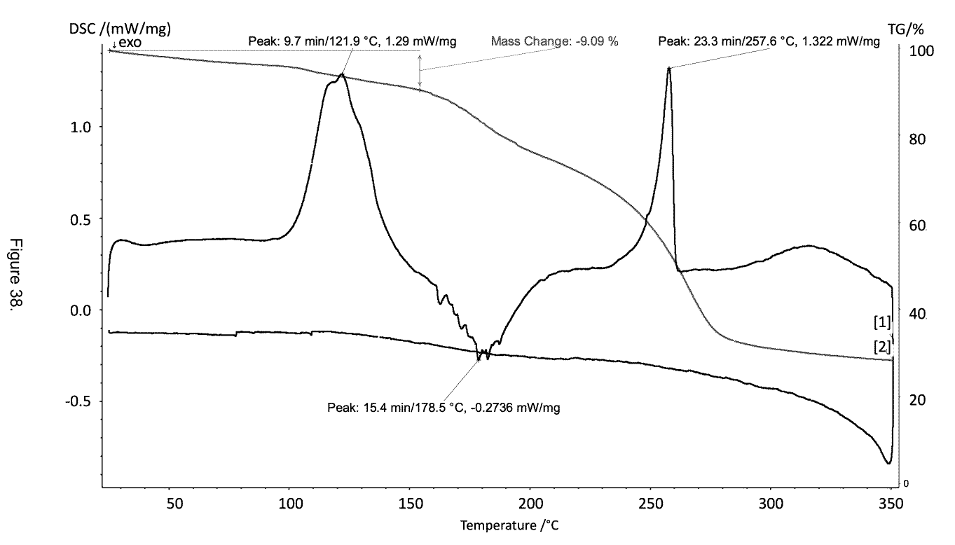

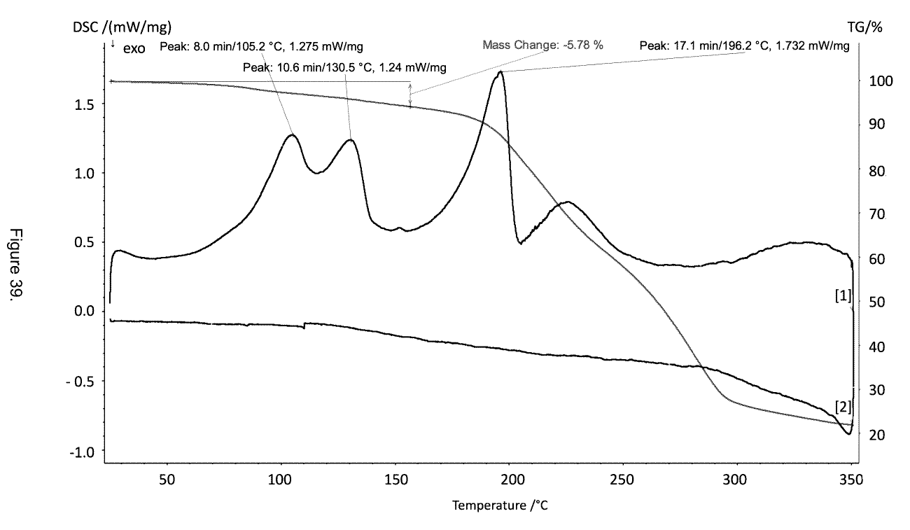

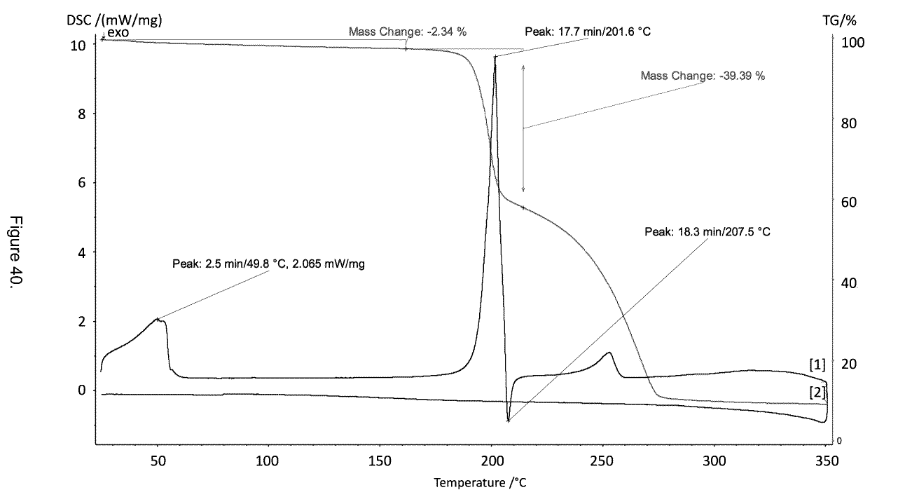

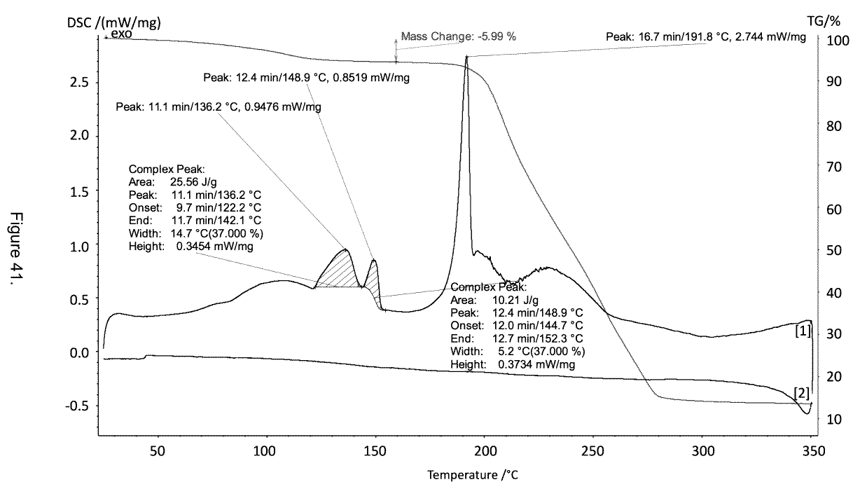

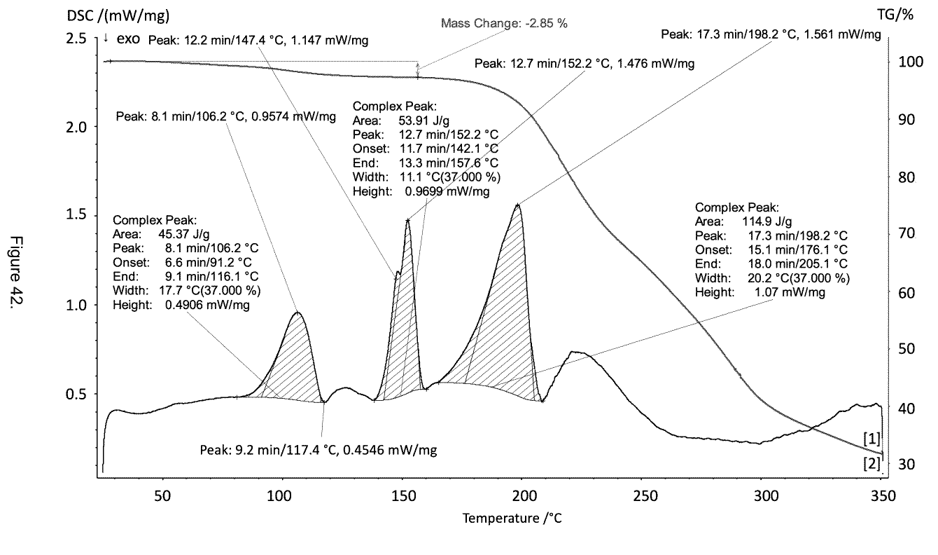

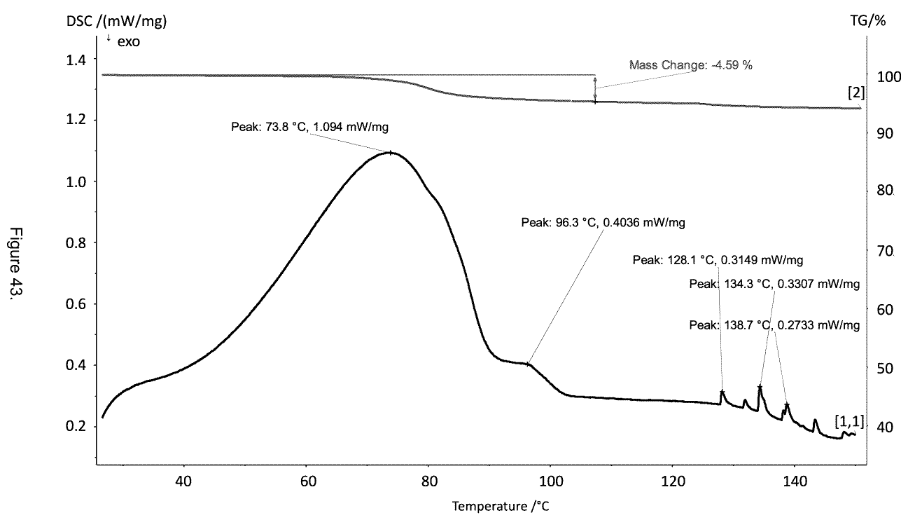

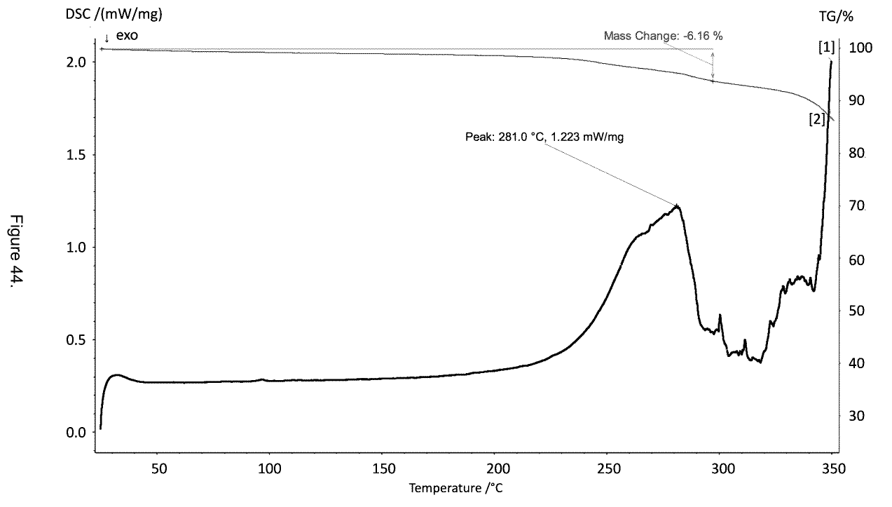

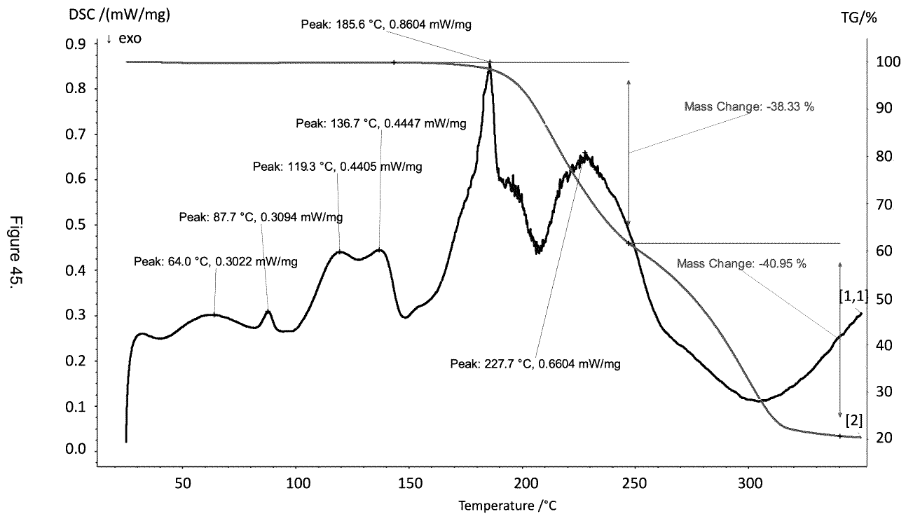

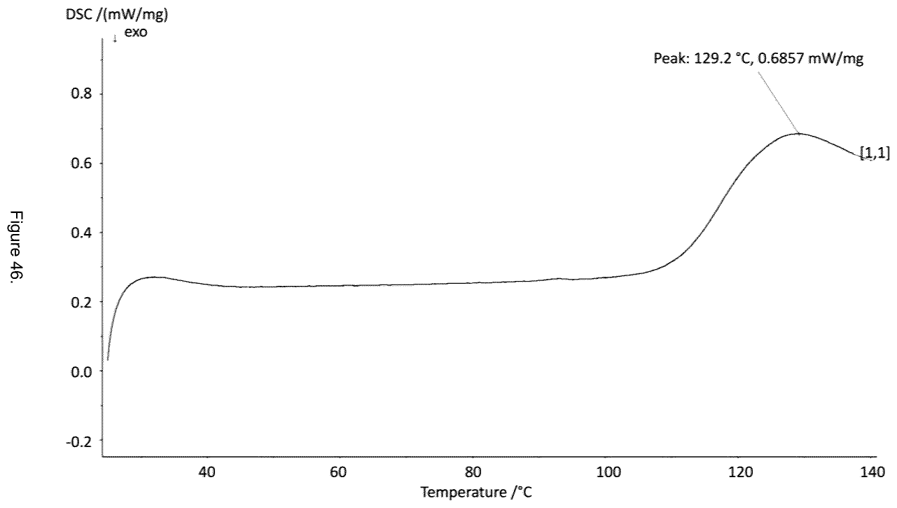

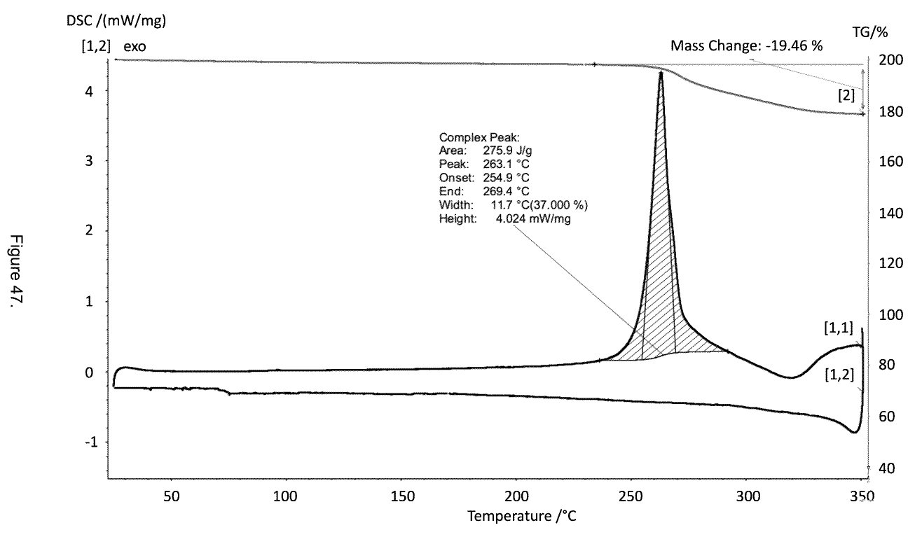

The thermal behavior (DSC and TGA) of the provided sample (ethHAR001) and all generated salt scNF01 to scNF12 are shown in the FIGS. 35-47. The results of the thermal investigations are summarized in table 8 below.

TABLE 8
Summary of the DSC and TGA results

| # | SOLID-CHEM | DSC | TGA |
|---|---|---|---|
| API | ethHAR001 | Onset 264.4° C. (endo) | mass loss beginning at |
| provided sample | | | 220° C. |
| scNF1 + API | ethHAR001GRI001 | Peak 105.1° C. (endo) | change in mass up to |
| succinic acid | | Peak 134.9° C. (endo) | 120° C. −4.6% |
| grinding | | Peak 148.2° C. (endo) | MV ab 160° C. |
| scNF2 | ethHAR001GRI002 | Peak 114.8° C. (endo) | change in mass up to |
| L-(+)-tartaric acid | | Peak 192.3° C. (endo) | 140° C. −9.3% |
| grinding | | | mass loss beginning at |
| | | | 200° C. |
| scNF6 | ethHAR001EVA014 | Peak 136.2° C. (endo) | change in mass up to |
| L-(+)-tartaric acid | | Peak 148.9° C. (endo) | 160° C. −6.0% |
| evaporation | | Peak 191.8° C. (endo) | mass loss beginning at |
| | | | 180° C. |
| scNF3 | ethHAR001GRI003 | Peak 121.9° C. (endo) | change in mass up to |
| L-ascorbic acid | | Peak 178.5° C. (exo) | 155° C. −8.1% |
| grinding | | Peak 257.6° C. (endo) | mass loss beginning at |
| | | | 165° C. |
| scNF4 + scNF7 | ethHAR001EVA016 | Peak 106.2° C. (endo) | change in mass up to |
| L-(−)-malic acid | | Peak 117.4° C. (exo) | 160° C. −2.9% |
| evaporation | | Peak 147.4° C. (endo) | mass loss beginning at |
| | | Peak 152.2° C. (endo) | 170° C. |
| | | Peak 198.2° C. (endo) | |
| scNFS | ethHAR001GRIOOS | Peak 49.8° C. (endo) | change in mass up to |
| citric acid | | Peak 201.6° C. (endo) | 160° C. −2.3% |
| grinding | | Peak 207.5° C. (exo) | mass loss beginning at |
| | | | 180° C. |
| scNF4 + API | ethHAR001GRI004 | Peak 105.2° C. (endo) | change in mass up to |
| L-(−)-malic acid | | Peak 130.5° C. (endo) | 160° C. −5.8% |
| grinding | | Peak 196.2° C. (endo) | mass loss beginning at |
| | | | 170° C. |
| scNF8 | ethHAR001EXP113 | Onset 79.6° C. (endo) | change in mass up to |
| methanesulfonic acid | | Onset 238.1° C. (endo) | 105° C. −4.4% |
| evaporation | | Cooling curve | mass loss beginning at |
| | | Peak 305° C. (exo) | 110° C. step at 190° C. |
| scNF9 | ethHAR001EXP114 | Peak 268.5° C. (endo) | change in mass up to |
| sulfuric acid | | Peak 281.0° C. (endo) | 240° C. −1.93% |
| evaporation | | | mass loss beginning at |
| | | | 240° C. |
| scNF10 | ethHAR001GRI014 | Peak 64.0° C. (endo) | mass loss beginning at |
| L-tartaric acid | | Peak 87.7° C. (endo) | 186° C. |
| grinding | | Peak 119.3° C. (endo) | |
| | | Peak 136.7° C. (endo) | |
| | | Peak 185.6° C. (endo) | |
| | | Peak 227.7° C. (endo) | |
| scNF11 | ethHAR001GRI015 | Peak 129.2° C. (endo) | — |
| L-ascorbic acid | | | |
| grinding | | | |
| scNF12 | ethHAR001EXP115 | Onset 254.8° C. (endo) | mass loss beginning at |
| phosphoric acid | | | 240° C. |
| evaporation | | | |

### Thermal Behavior of the Different Salts

In order to study the thermal behavior of the different salts TGA/DSC measurements were performed (see table 8 above).

It can be observed, that all salts show changes in mass up to 160° C. In contrast Intending to study the ‘nature’ of the mass loss a so-called preparative DSC measurement was performed. Therefore, a further DSC measurement at different temperatures were carried out followed by a powder X-Ray diffraction measurement after cooling the sample. The results and defined temperatures are summarized in table 10.

TABLE 10
Results of the preparative DSC experiments

| | | DSC | XRPD |
|---|---|---|---|
| ETH | SOLID-CHEM | tempering up to | pattern change |
| scNF1 | ethHAR001EVA013 | T₁ = 124° C. | scNF1 |
| succinic acid | | T₂ = 160° C. | mostly |
| evaporation | | | amorphous |
| scNF2 | ethHAR001GRI002 | T = 140° C. | mostly |
| L(+)-tartaric | | | amorphous |
| acid | | | |
| grinding | | | |
| scNF6 | ethHAR001EVA014 | T = 160° C. | new pattern |
| L(+)-tartaric | | | |
| acid | | | |
| evaporation | | | |
| scNF3 | ethHAR001GRI003 | T₁ = 140° C. | mostly |
| L-ascorbic | | T₂ = 225° C. | amorphous |
| acid | | | mostly |
| grinding | | | amorphous |
| scNF4 + API | ethHAR001GRI004 | T = 140° C. | mostly |
| L-(−)-malic | | | amorphous |
| acid | | | |
| grinding | | | |
| scNF4 + scNF7 | ethHAR001EVA016 | T₁ = 138° C. | mostly |
| L-(−)-malic | | T₂ = 160° C. | amorphous |
| acid | | | amorphous |
| evaporation | | | |
| scNF8 | ethHAR001EXP113 | T = 150° C. | scNF8 |
| methanesulfonic | | | |
| acid | | | |
| evaporation | | | |
| scNF9 | ethHAR001EXP114 | T = 210° C. | scNF9 |
| sulfuric acid | | | |
| evaporation | | | |

### Spectroscopy

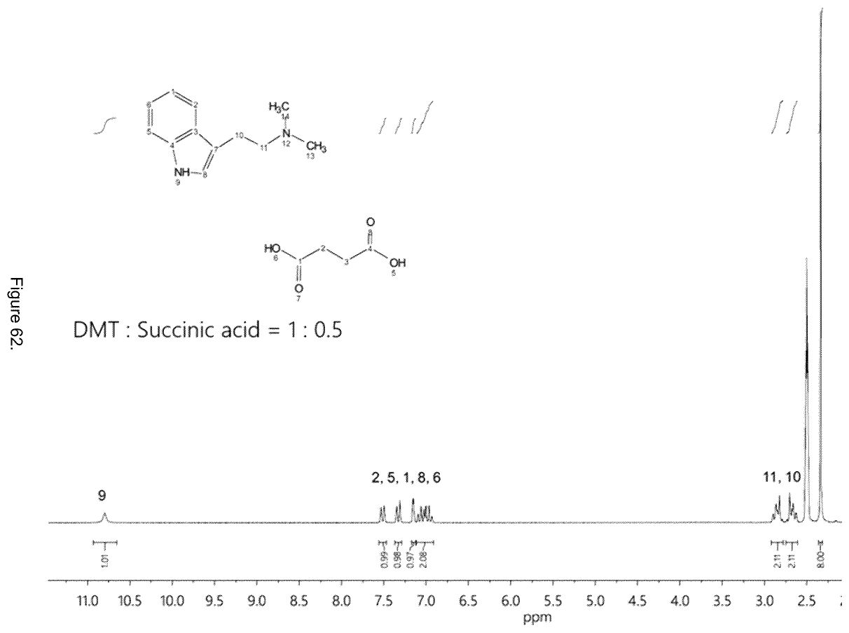

Since all generated salts showed insufficient thermal properties the focus was set to the phosphoric acid salt. All work on other experiments was discontinued. To investigate whether the generated salt is a hydrogen phosphate or a dihydrogen phosphate, several spectra of the generated phosphoric acid salt (ethHAR001EXP115-scNF12) and potassium-hydrogen phosphate as well as potassium-dihydrogen phosphate where collected and compared to each other. The results are shown in FIG. 61.

The Figure shows that the experiment ethHAR001EXP115 forms a halo-like curve between 500-1250 cm⁻¹ which corresponds to the behavior of the spectrum of a potassium-hydrogen phosphate salt. Therefore, it can be concluded that the formed salt is also a hydrogen phosphate salt and not a dihydrogen phosphate. All significant signals of dihydrogen-phosphate cannot be detected in the formed salt.

### Characterization

The characterizations of the newly discovered solid phases were performed by the following methods: XRPD, polarization microscopy (PLM), DSC, TGA, IR as well as ¹H-NMR.

Within the framework of the dynamic screening process, a complete characterization of new entities was discontinued, if previous test results of the XRPD method and the method combination DSC/TGA have already shown that the new forms cannot be used for subsequent galenic processes.

The following table 11 provides an overview of the characterization methods utilized for the new solid phases.

TABLE 11
Overview characterization methods of new harmine solid states

| | Salt-Former | XRPD | PLM | DSC | TGA | IR | ¹H-NMR |
|---|---|---|---|---|---|---|---|
| SC-NF0 | | ✓ | ✓ | ✓ | ✓ | ✓ | ✓ |
| SC-NF1 | Succinic acid | ✓ | ✓ | ✓ | ✓ | ✓ | ✓ |
| SC-NF2 | L(+)-Tartaric acid | ✓ | ✓ | ✓ | ✓ | ✓ | ✓ |
| SC-NF3 | L-Ascorbic acid | ✓ | ✓ | ✓ | ✓ | ✓ | ✓ |
| SC-NF4 | L(−)-Malic acid | ✓ | ✓ | ✓ | ✓ | ✓ | ✓ |
| SC-NF5 | Citric acid | ✓ | ✓ | ✓ | ✓ | ✓ | ✓ |
| SC-NF6 | L(+)-Tartaric acid | ✓ | | ✓ | ✓ | | ✓ |
| SC-NF7 + SC-NF4 | L(−)-Malic acid | ✓ | | ✓ | ✓ | | ✓ |
| SC-NF8 | Methanesulfonic acid | ✓ | | ✓ | ✓ | | ✓ |
| SC-NF9 | Sulfuric acid | ✓ | | ✓ | ✓ | | ✓ |
| SC-NF10 | L(+)-Tartaric acid | ✓ | | ✓ | ✓ | | |
| SC-NF11 | L-Ascorbic acid | ✓ | | ✓ | ✓ | | |
| SC-NF12 | Phosphoric acid | ✓ | | ✓ | ✓ | ✓ | ✓ |
| SC-NF13 | L(+)-Tartaric acid | ✓ | | | | | |
| SC-NF14 | L-Ascorbic acid | ✓ | | | | | |

### Further Analysis

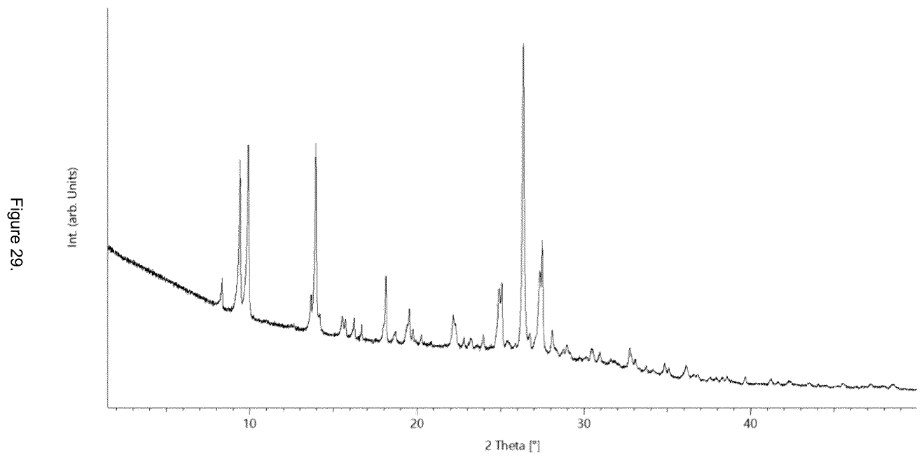

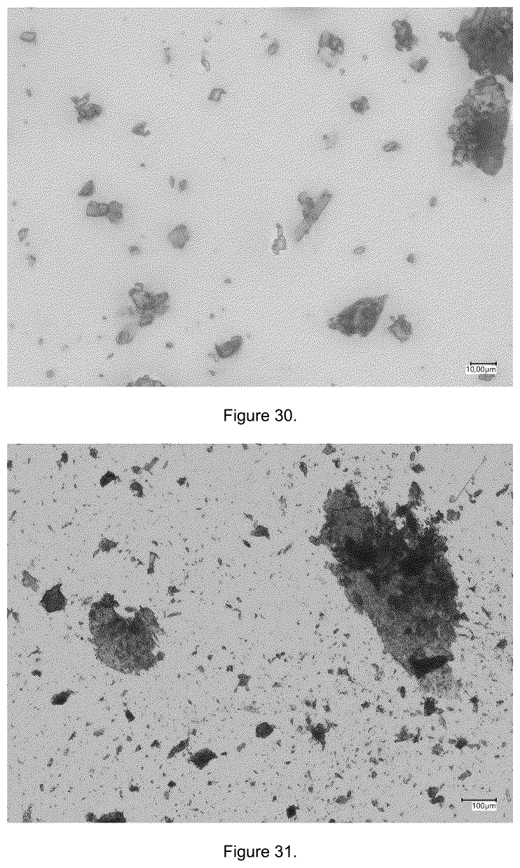

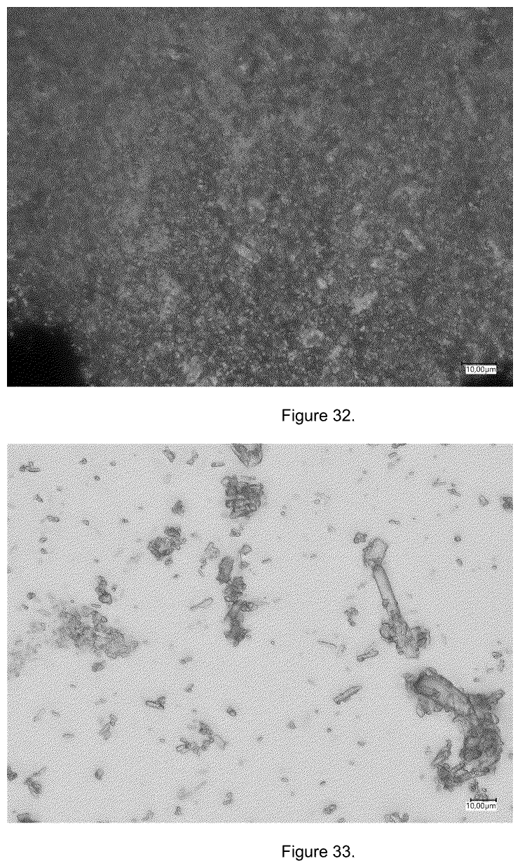

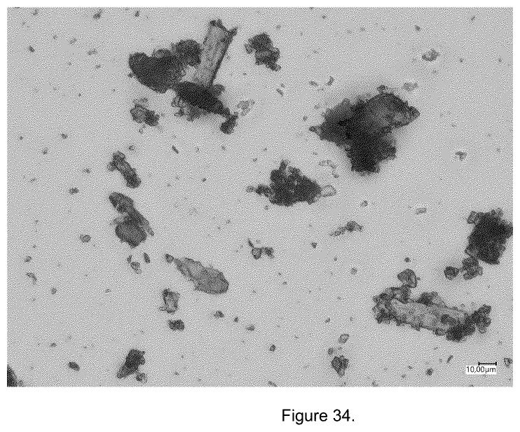

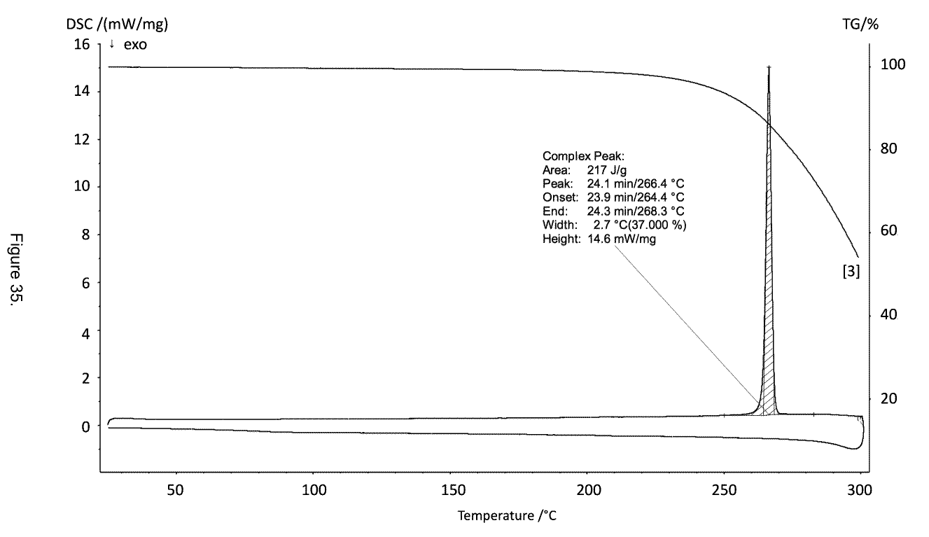

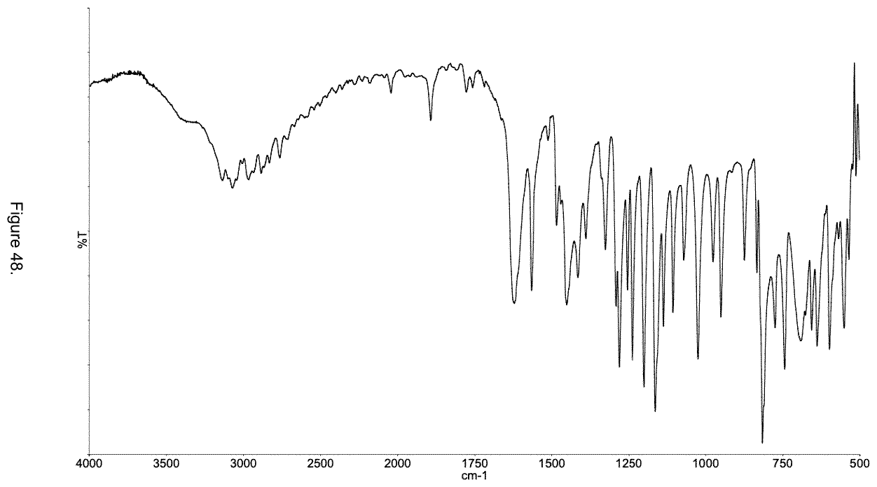

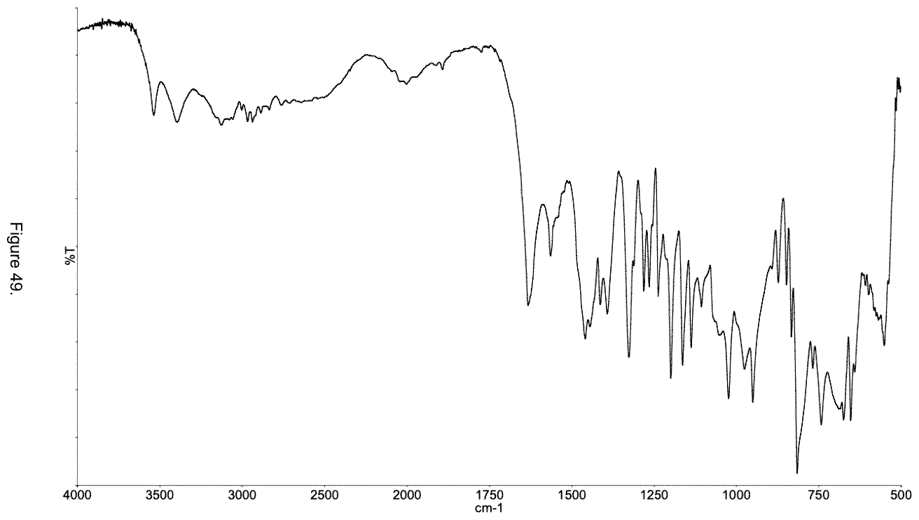

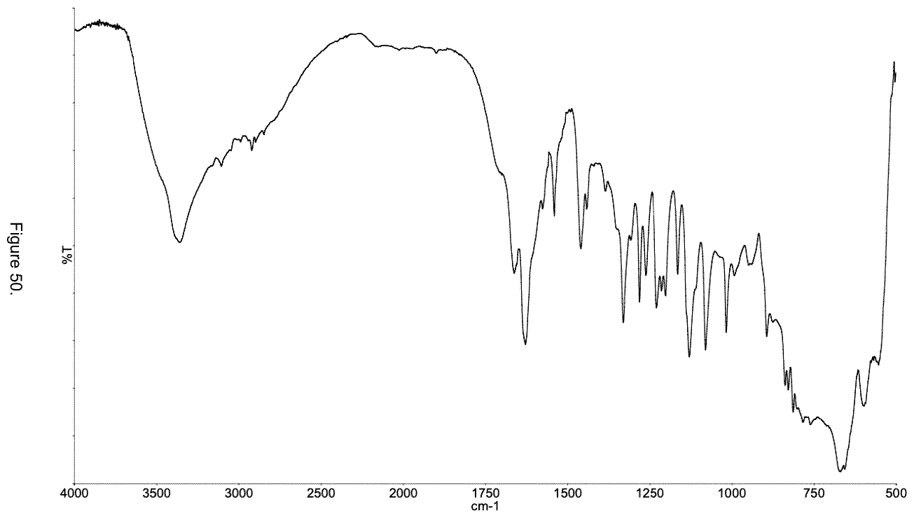

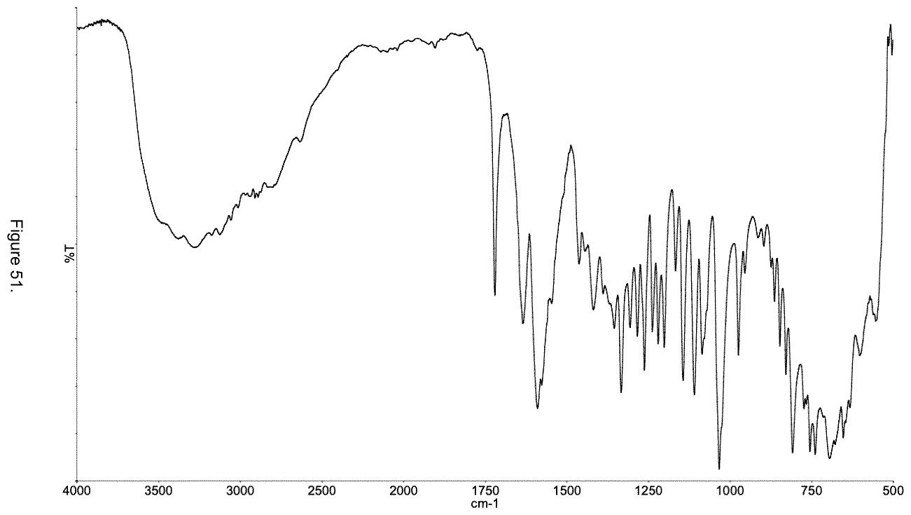

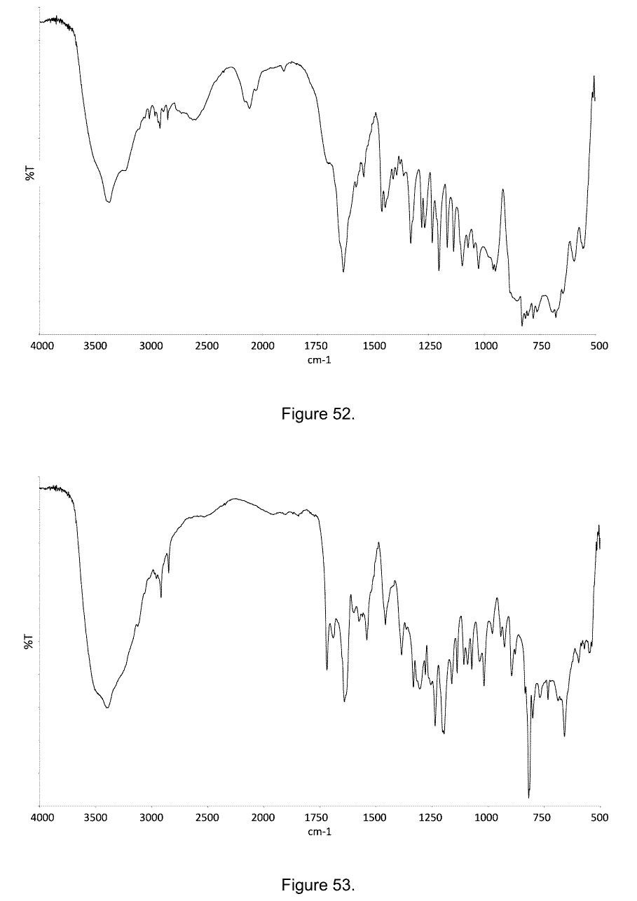

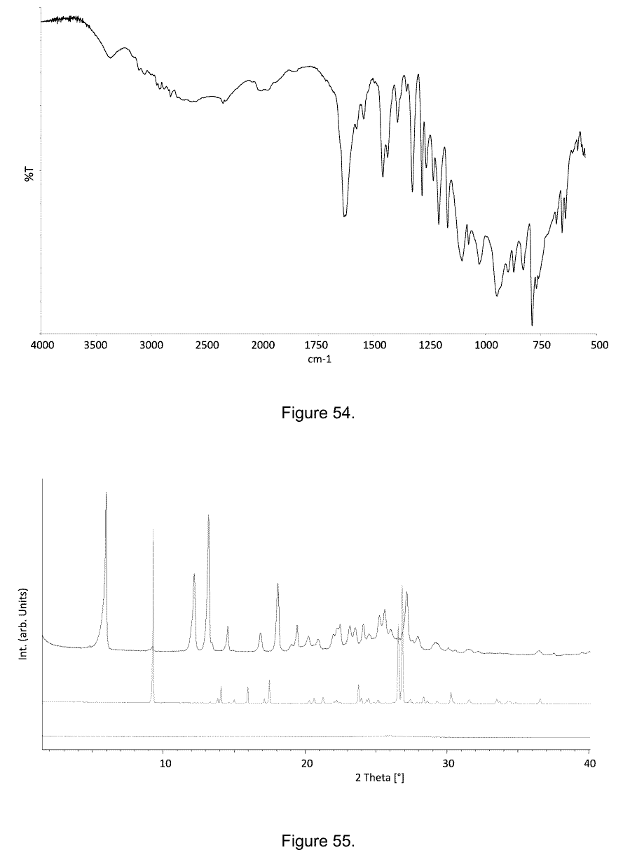

Microscopic picture of differen harmine salts are shown in FIGS. 30-34. IR spectra of different harmine salts are shown in FIGS. 48-54.

### Salt Screening-Conclusion

From the active ingredient Harmine (SOLID-CHEM: ethHAR001), 14 new solid-state entities were obtained using a total of eight different salt formers.

The screening results show that salt formation experiments using both strong and weak acids lead to Harmine salts. However, further investigations on the salts of weak acids reveal that these new Harmine salts exhibit numerous thermal events in the low temperature range, which indicate the release of water or solvent molecules of the formed hydrates or solvates. Furthermore, the complex thermograms indicate that decomposition reactions of the salt formers occur (cf. Table 5). Therefore, the use of further weak acids was omitted in the screening and instead strong acids such as Methanesulfonic acid, Sulfuric acid and Phosphoric acid were used as salt formers. The salts obtained, of the three strong acids, were also thermoanalytically analyzed.

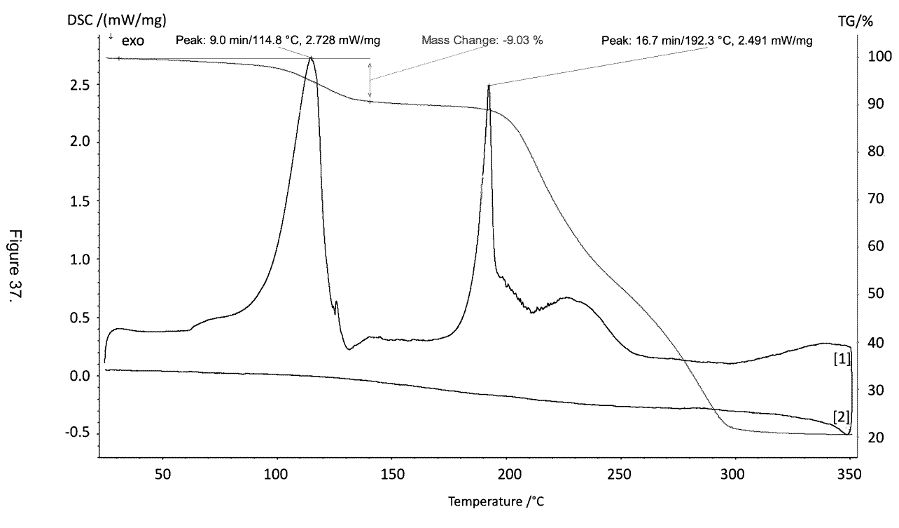

Of all three salts formed from strong acids, only Harmine-Phosphate shows a promising DSC thermogram with one endothermic signal at 254.8° C., which can be assigned to the melting of the salt. The detected mass loss only starts at 240° C. (see FIG. 36), further solubility studies were carried out on the Phosphate salt of Harmine. Compared with the Harmine-HCl salt, however, these do not show any better solubility properties. Thus, to further investigate solubility enhancing strategies, the present inventors performed a screening of different harmine compositions described in the next section.

### Screening Compositions Containing Harmine and Different Acids

The polymorph- and salt screening experiments described hereinabove, did not yield solid state forms of harmine with improved stability compared to the standard harmine HCl. Thus, the present inventors performed an additional screening, in which different compositions of harmine were manufactured and their solubility in water was examined.

To this end, aqueous solutions containing harmine and acids, such as D-glucuronic acid, D-galacturonic acid, D-gluconic acid, tartaric acid, succinic acid, citric acid, mucic acid, ascorbic acid, acetic acid, aspartic acid and glutamic acid were formed and tested for solubility. To this end, in a first experiment 1 g of harmine freebase was added to 5 g of dH2O to yield a final concentration of 20% (w/w). Then, the acids mentioned above were individually added to the solution in different molar rations (e.g. 1:1 or 2:1) and it was examined, whether the acids can solubilize harmine freebase by protonation or complex formation.

### SUMMARY OF THE RESULTS

Tartaric, succinic acid, citric, mucic, glutamic and ascorbic acid were not able to solubilize harmine. All these acids formed a paste with the same color as harmine. On the other hand, D-glucuronic acid, D-galacturonic acid, D-gluconic acid and yield a solubility higher than 15%. It is thought that the high solubility is driven by the sugar acid, a strong interaction occurs between harmine and the acid leading to a charge transfer complex. Glucuronic acid gives the highest solubility.

The compositions of the investigated formulations and their solubility are summarized in the following table 12:

TABLE 12
Investigated compositions and their solubility

| Formula | Composition | Solubility |
|---|---|---|
| S50 | GLUCURONIC acid | Solution |
| | Molar ratio HAR/GLUCU (1:1) | transparent |
| | Dissolution: HAR/H2O (w/w): 25% | (yellow), no |
| | HAR 250 mg GLUCU 234 mg 1.0 g H2O | particles, |
| | | complete |
| | | dissolution |
| S230 | Galacturonic acid | Transparent, |
| | Molar ratio HAR/GALA (1:1) | complete |
| | Dissolution: HAR/H2O (w/w): 21% | dissolution, |
| | HAR 212 mg GALA 212 mg 1.0 g H2O | |
| S220 | Aspartic acid (ASP) | Transparent, |
| | Molar ratio HAR/ASP (1:1) | total dissolution |
| | Dissolution: HAR/H2O (w/w): 21% | |
| | HAR 212 mg ASP 133 mg 1.0 g H2O | |
| S280 | Aspartic acid (ASP) Reproduction | Transparent, |
| | Molar ratio HAR/ASP (1:1) | total dissolution |
| | Dissolution: HAR/H2O (w/w): 21% | |
| | HAR 212 mg ASP 133 mg 1.0 g H2O | |
| S70 | Galacturonic acid (GALA) + Glycine (GLY) | Solution |
| | Molar ratio HAR/GALA/GLY (1:1.5:1) | transparent |
| | Dissolution: HAR/H2O (w/w): 21% | (yellow), no |
| | HAR 212 mg GLY 75 mg GALA 294 mg 1.0 g H2O | particles, |
| | | complete |
| | | dissolution |
| S80 | UREA + Acetic acid (ACE) | Solution |
| | Molar ratio HAR/UREA/ACE (1:1:1) | transparent |
| | Dissolution: HAR/H2O (w/w): 25% | (yellow), no |
| | HAR 250 mg ACE 75 mg UREA 75 mg 1.0 g H2O | particles, |
| | | complete |
| | | dissolution |
| S270 | Glutatamic acid (GLU) reproduction | No complete |
| | Molar ratio HAR/ASP (1:1) | dissolution |
| | Dissolution: HAR/H2O (w/w): 21% | |
| | HAR 212 mg GLU 147 mg 1.0 g H2O | |
| S92 | Succinic acid + UREA + Acetic acid | Liquid but no |
| | Molar ratio HAR/SUC/UREA (1:0.5:0.5) | dissolution, lots |
| | Dissolution: HAR/H2O (w/w): 21% | of particles |
| | HAR 212 mg SUC 59 mg UREA 30 mg ACE 30 mg 1.0 g H2O | |
| S102 | Glucuronic acid (GLUCU) + Galacturonic acid (GALA) | Transparent |
| | Molar ratio HAR/GLUCU/GALA (1:1:1) | solution |
| | Dissolution: HAR/H2O (w/w): 25% | (yellow), no |
| | HAR 250 mg GLUCU 114 mg GALA mono 132 mg 60 mg ACE | particles, |
| | 1.0 g H2O | complete |
| | | dissolution |
| S110 | Glucose + Acetic acid | Soluble |
| | Molar ratio HAR/Glucose/ACE (1:1:1.5) | |
| | Dissolution: HAR/H2O (w/w): 21% | |
| | HAR 212 mg Glucose 180 mg ACE 90 mg 1.0 g H2O | |
| S121 | Glucose + Acetic acid | Complete |
| | Molar ratio HAR/Glucose/ACE (1:1:1) | dissolution |
| | Dissolution: /HAR/H2O (w/w): 21% | |
| | HAR 212 mg Glucose 180 mg ACE 120 mg 1.0 g H2O | |
| S130 | UREA + Acetic acid | Dissolution, |
| | Molar ratio HAR/UREA/ACE (1:1:1.5) | transparent |
| | Dissolution: HAR/H2O (w/w): 21% | |
| | HAR 212 mg UREA 60 mg ACE 90 mg 1.0 g H2O | |
| S142 | GLUTAMIC Acid + ACETIC acid | Liquid but no |
| | Molar ratio HAR/GLUTA/ACE (1:1:2) | dissolution, Lots |
| | Dissolution: HAR/H2O (w/w): Target 25% | of particles |
| | HAR 250 mg GLUTA 173 mg ACE 150 mg 1.0 g H2O | |
| S150 | CITRIC Acid + Menthol | Formation of a |
| | Molar ratio HAR/CIT/MEN (1:0.5:0.5) | white paste |
| | Dissolution: HAR/H2O (w/w): Target 25% | |
| | HAR 250 mg CIT 123 mg MEN 92 mg 1.0 g H2O | |
| S170 | MALIC Acid | At the beginning |
| | Molar ratio HAR/MAL (1:1) | soluble, with |
| | Dissolution: HAR/H2O (w/w): 20% | formation of |
| | HAR 250 mg MAL 158 mg 1.25 g H2 | yellow liquid, |
| | | after 5 min it |
| | | turns into a |
| | | white paste |
| S180 | MALIC Acid | Formation of a |
| | Molar ratio HAR/MAL (1:1.5) | white paste |
| | Dissolution: HAR/H2O (w/w): 25% | |
| | HAR 250 mg MAL 237 mg 1.0 g H2O | |
| S190 | GLUCURONIC Acid | Transparent, |
| | Molar ratio HAR/GLUCU (1:1) | total dissolution |
| | Dissolution: HAR/H2O (w/w): 25% | |
| | HAR 250 mg GLUCU 228 mg 1.0 g H2O | |
| S200 | Deep eutectic: Malic acid/Fructose | Transparent, |
| | Molar ratio HAR/FRU/MAL (1:0.5:0.5) | total dissolution |
| | Dissolution: HAR/H2O (w/w): 21% | |
| | HAR 212 mg FRU 90 mg MAL 67 mg 1.0 g H2O | |
| S210 | Aspartic acid and acetic acid | Liquid, |
| | Molar ratio HAR/ASP/ACE (1:0.5:0.5) | dissolution |
| | Dissolution: HAR/H2O (w/w): 25% | almost |
| | HAR 250 mg ASP 79 mg ACE 75 mg 1.0 g H2O | complete, some |
| | | particles at the |
| | | bottom |
| S240 | Arabinose (ARA) | No dissolution |
| | Molar ratio HAR/ARA (1:1) | |
| | Dissolution: HAR/H2O (w/w): Target 21% | |
| | HAR 212 mg ARA 150 mg 1.0 g H2O | |
| S250 | Deep eutectic: Malic acid MAL/Arabinose ARA | Transparent, |
| | Molar ratio HAR/ARA/MAL (1:0.5:0.5) | dissolution but |
| | Dissolution: HAR/H2O (w/w): 21% | not stable |
| | HAR 212 mg ARA 75 mg MAL 67 mg 1.0 g H2O | |

In addition, the following table 13 below shows solubility of different compositions of harmine with an uronic acid, whereas the composition of harmine and glucuronic acid shows the highest solubility:

TABLE 13
Additionally investigated compositions and their solubility

| | | Molar - | | | |
|---|---|---|---|---|---|
| | | Ratio | | | |
| | | HAR:ACI | Mass | | |
| | | (harmine- | fraction | | |
| Sample | Raw data | acide) | HAR:H2O | Picture | Comment |
| S1 | 0.45 g HAR | 1:1 | 1:3 | | Formation of |
| | 0.92 GLUCO | | 33% | | complex, |
| | 0.9 g H2O | | | | colour |
| | | | | | change, |
| | | | | | solution is |
| | | | | | not fully |
| | | | | | transparent |
| S2 | 0.25 g HAR | 1:1 | 1:5 | | Formation of |
| | 0.25 g GALA | | 20% | | complex, |
| | 1.26 g H2O | | | | colour |
| | | | | | change, |
| | | | | | solution is |
| | | | | | not fully |
| | | | | | transparent |
| S3 | 0.25 g HAR | 1:1 | 1:5 | | Formation of |
| | 0.25 g MUC | | 20% | | a paste. |
| | 1.25 g H2O | | | | No complex |
| | | | | | formation |
| S4 | 0.25 g HAR | 1:1 | 1:5 | | Formation of |
| | 0.46 g sol | | 20% | | complex, |
| | GLUCO | | | | colour |
| | 1 g H2O | | | | change, |
| | | | | | solution is |
| | | | | | not fully |
| | | | | | transparent |
| S5 | 0.25 g HAR | 1:2 | 17% | | Solution |
| | 1.02 g sol | | | | fully |
| | GLUCO | | | | transparent. |
| | 1 g H2O | | | | Complex is |
| | | | | | fully |
| | | | | | dissolved |
| S6 | 0.25 g HAR | 1:1 | 1.5 | | Formation of |
| | 0.46 g sol | | 20% | | complex, |
| | GLUCO | | | | colour |
| | 1 g H2O | | | | change, |
| | | | | | solution is |
| | | | | | not fully |
| | | | | | transparent |
| S7 | 0.26 g HAR | 1:1 | 17% | | Formation of |
| | 0.25 g GALA | | | | complex, |
| | 1.5 g H2O | | | | colour |
| | | | | | change, |
| | | | | | solution is |
| | | | | | not fully |
| | | | | | transparent |
| S8 | 0.25 g HAR | 1:2 | 17% | | Solution |
| | 0.50 g GALA | | | | fully |
| | 1.5 g H2O | | | | transparent. |
| | | | | | Complex is |
| | | | | | fully |
| | | | | | dissolved |
| S9 | 0.45 g HAR | 1:2 | 28.3% | | Solution is |
| | 1.38 GLUCO | | | | almost |
| | 0.1.59 g H2O | | | | transparent |
| S10 | 0.45 g HAR | 1:2 | 25% | | Solution |
| | 1.38 GLUCO | | | | fully |
| | 0.1.79 g H2O | | | | transparent. |
| | | | | | Complex is |
| | | | | | fully |
| | | | | | dissolved |

Further tested were mixtures and salts, as described in the following.

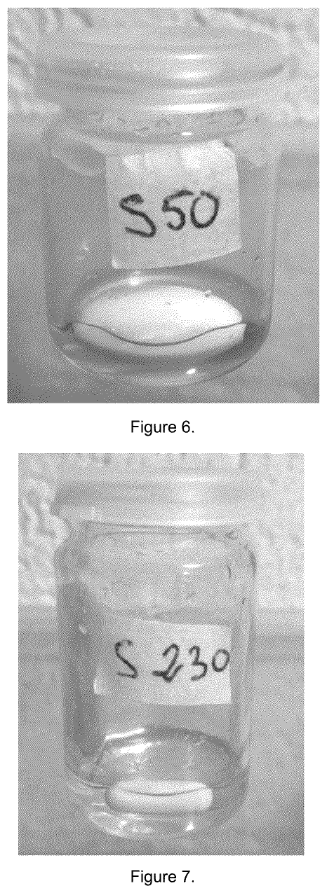

A composition comprising harmine and glucuronic acid in the molar ratio of 1:1 was dissolved to 25% in water (by combining 250 mg of harmine FB, 234 mg of glucuronic acid and 1.0 g of water). Yellow transparent solution without particles has been obtained, indicative of complete dissolution of harmine (or its formed salt). The result of dissolution is shown in FIG. 6.

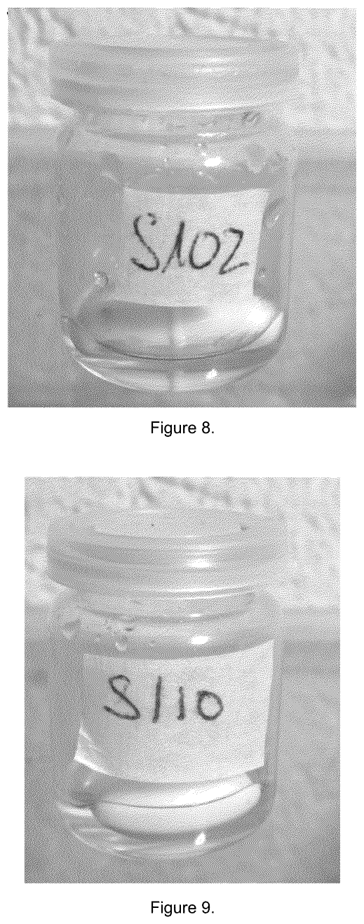

A composition comprising harmine and galacturonic acid in molar ratio of 1:1 was dissolved to 21% in water (by combining 212 mg harmine free base, 212 mg galacturonic acid and 1.0 g water). The so obtained solution was transparent, indicative of complete dissolution (FIG. 8).

A composition comprising harmine, glucuronic acid and galacturonic acid in molar ratio of 1:1:1 was dissolved to 25% in water by combining 250 mg harmine FB, 114 mg glucuronic acid, 132 mg galacturonic acid, 60 mg acetic acid and 1.0 g water. A yellow, transparent solution without particles, indicative of complete dissolution, has been obtained (FIG. 8).

A composition comprising harmine, glucose and acetic acid in a molar ratio of 1:1:1.5 was dissolved to 21% in water, by combining 212 mg harmine FB, 180-mg glucose, 90 mg acetic acid and 1.0 g water. As shown in FIG. 9, the composition is soluble in water.

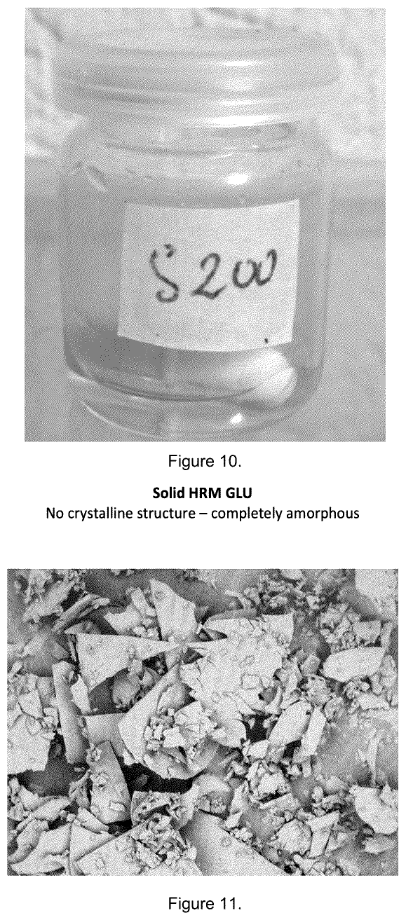

A composition comprising harmine, fructose and malic acid in a molar ratio of 1:0.5:0.5 was dissolved to 21% in water by combining 212 mg harmine FB, 90 mg fructose, 67 mg malic acid and 1.0 g water. A transparent solution has been obtained (FIG. 10).

### Conclusion—Screening Compositions Containing Harmine and Different Acids

Accordingly, new compositions and/or salts of harmine that are highly soluble in water have been surprisingly identified by the present inventors.

Accordingly, it has been shown that certain compositions comprising harmine and as described in claim 1 show improved solubility in comparison to harmine free base or harmine hydrochloride. Most promising, compositions containing harmine and glucuronic acid yielded very high solubility. For this reason, harmine glucuronate was formed and characterized as described in the previous salt screening section under “characterization”. In the following, the formation of harmine glucuronate and the analysis of its characteristics is described:

### Formation of Harmine Glucuronate

Weigh out harmine and an equimolar amount of glucuronic acid and place both substances in a beaker with magnetic stirrer. Add deionized water (diH2O) while stirring until a transparent solution is formed. Typically, harmine concentrations of >20% are readily feasible, i.e., 1 g harmine (+ the equimolar amount of glucuronic acid) is readily soluble in 5 mL water (i.e., 5 mL final volume). For example, approximately 3 mL of diH2O can be added with stirring until the solids are dissolved. Subsequently, a syringe is used to make up to the final volume of 5 mL with diH2O. This solution is then stirred again until a homogeneous solution is obtained. The transparent solution is evaporated on the RotaVap until a completely dry precipitate is obtained.

### Solubility Testing

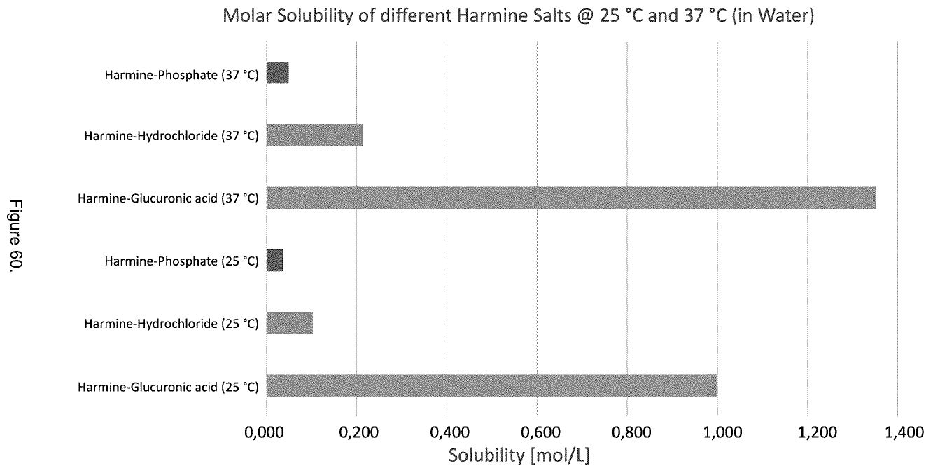

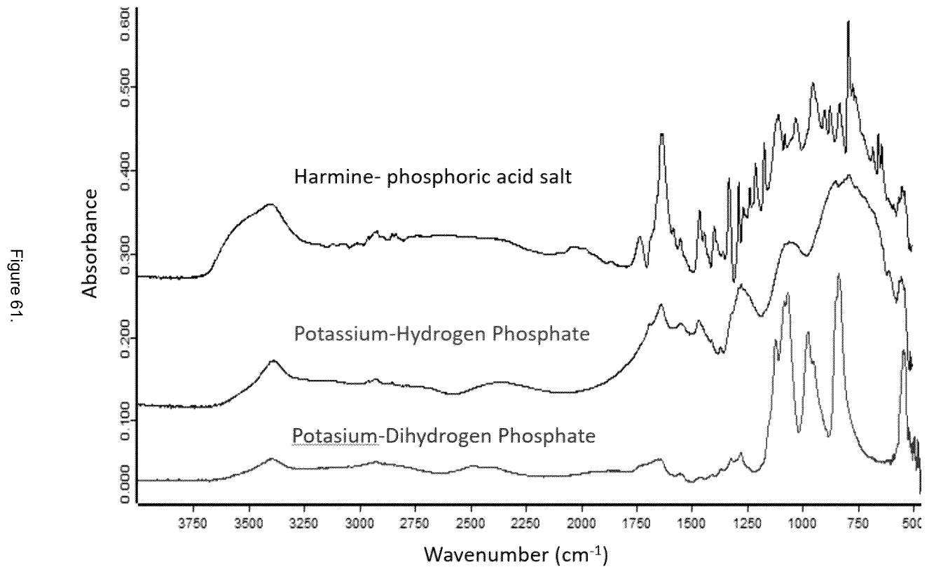

The solubility of harmine phosphate is significantly worse than that of hydrochloride. At least when the data in water are considered. The gravimetric solubility of the harmine glucuronate, on the other hand, is about eight times that of the harmin hydrochloride at 25° C. Thus, harmine glucuronate shows extremely improved solubility compared to the two salts of harmine. This property could be additionally enhanced by the amorphous structure of the precipitate. The solubilities are summarized in the table 14 below and in FIGS. 59 and 60:

TABLE 14
Comparison of the solubility of harmine phosphate, harmine HCl and harmine
glucuronate in H2O and EtOH, at 25° C. and 37° C.

| | Solubility at | | Solubility at | | Solubility at | | Solubility at | |
|---|---|---|---|---|---|---|---|---|
| Salt | 25° C. (H2O) | | 37° C. (H20) | | 25° C. (EtOH) | | 37° C. (EtOH) | |
| Harmine | 11.04 | mg/mL | 12.92 | mg/mL | 0.58 | mg/mL | 0.55 | mg/mL |
| Phosphate | | | | | | | | |
| Harmine HCl | 24.92 | mg/mL | 53.08 | mg/mL | 6.79 | mg/mL | 7.95 | mg/mL |
| Harmine | 405.95 | mg/mL | 549.52 | mg/mL | 4.25 | mg/mL | 7.93 | mg/mL |
| Glucuronate | | | | | | | | |

### NMR Analysis of Harmine Glucuronate

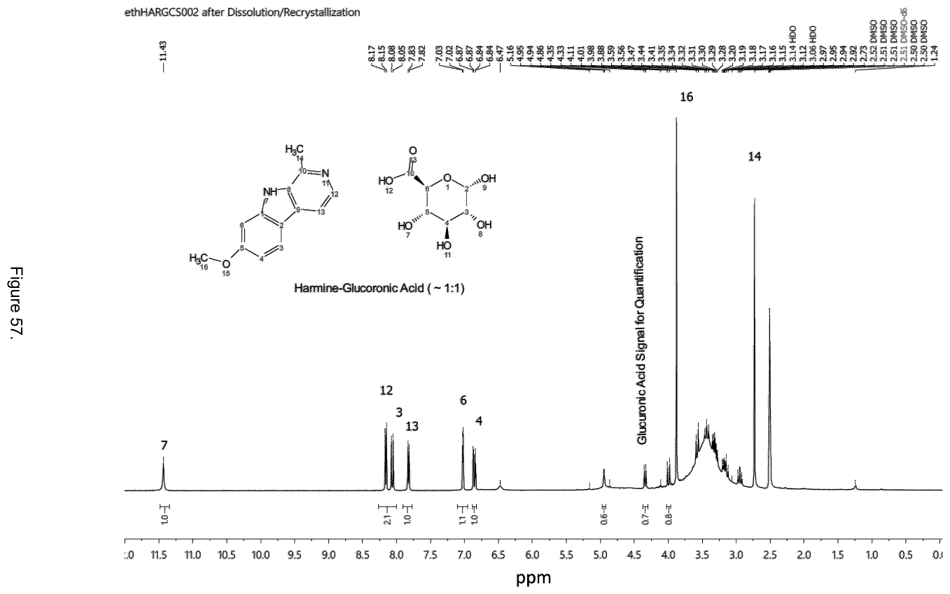

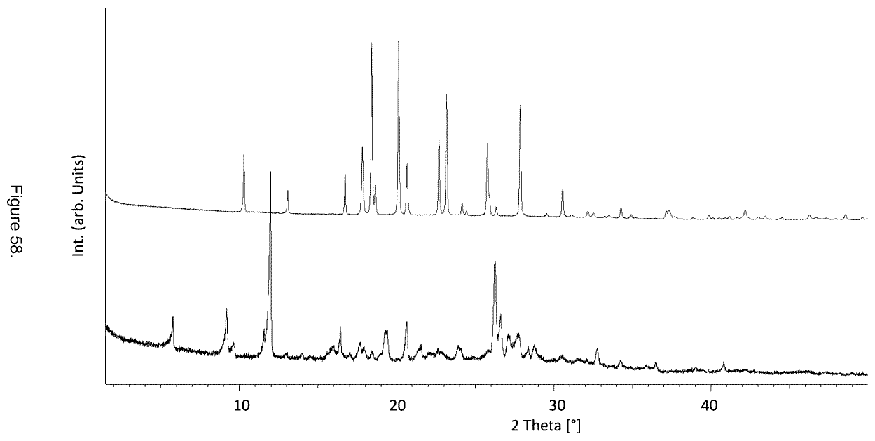

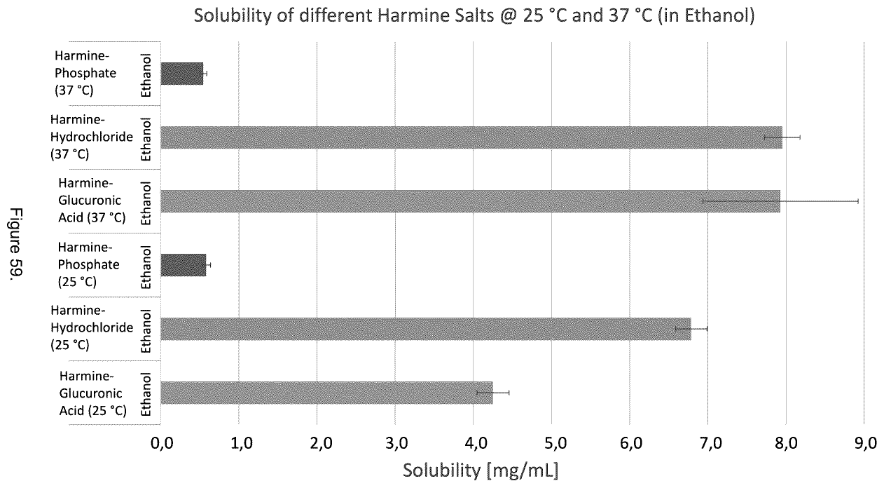

The NMR analysis of harmine glucuronate shows that the molar ratio of harmine and glucuronic acid is maintained. Furthermore, a new powder diffractogram is detected after recrystallization, which could indicate salt or cocrystal formation. Here, salt formation is more likely since the ΔpKs value of both compounds is 4.27. To confirm this result, an additional powder diffractogram of the pure glucuronic acid should be recorded for comparison. The NMR of harmine glucuronate is shown in FIGS. 56 and 57.

### PXRD Analysis of Harmine Glucuronate

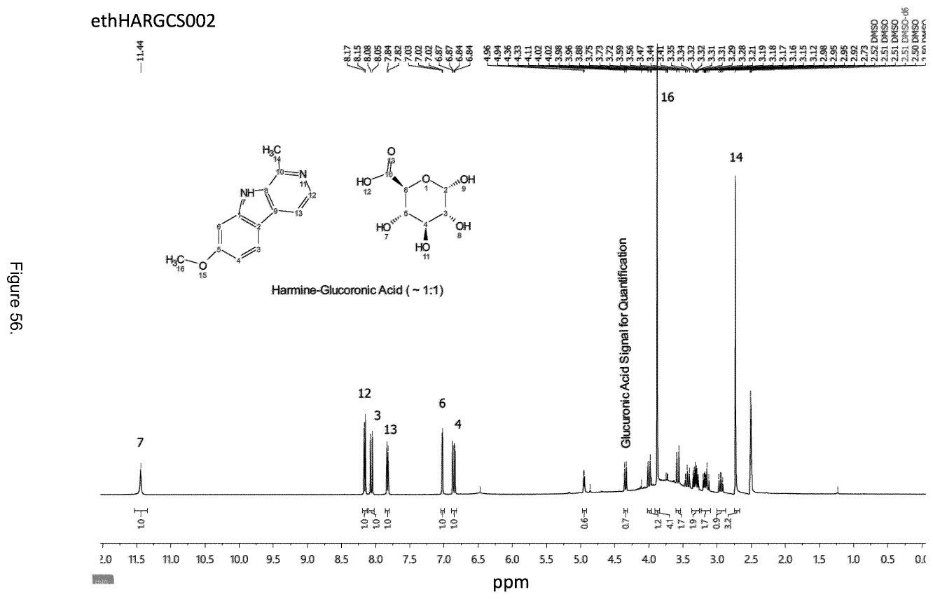

The PXRD of harmine glucuronate points towards a full amorphic structure of harmine glucuronate (FIG. 55+58), which may also explain the high solubility. The electron microscopic picture of harmine glucuronate support the assumption of a full amorphic structure (FIG. 11).

Part 2: Development of Oromucosal Dosage Forms of Harmine with Improved Bioavailability, Reduced First-Pass Metabolism and Improved Palatability

Goal of the study. In this study we aimed at identifying sublingual harmine formulations with favorable 1) pharmaceutical properties (e.g. physical/chemical stability; scalability of the manufacturing process), 2) pharmacokinetic properties (high bioavailability, low inter-/intrasubject plasma value variability) and 3) patient compliance (e.g. palatability (e.g. bitterness), easy handling). The Formulations are summarized in the Table 15 below and explained in the following section.

TABLE 15
Summary of the developed harmine sublingual formulations F1-F7.

| | Harmine solid | | |
|---|---|---|---|
| # | state form | Dosage form | Test species |
| F1 | Harmine freebase | Sublingual powder | Human |
| F2 | Harmine | Sublingual powder | Human |
| | Hemifumarate | | |
| F3 | Harmine HCl | Melting tablet | Human |
| | | (orodispersible tablet) | |
| F4 | Harmine glucuronate | Sublingual drops | Dog |
| | DMT Hemisuccinat | | |
| F5 | Harmine HCl | Fast Disintegrating Tablet | not tested |
| | | (formulated using TIP) | |
| F6 | Harmine HCl | Fast Disintegrating Tablet | Human |
| | | (formulated using TIP) | |
| F7 | Harmine glucuronate | Fast Disintegrating Tablet | to be tested |
| | DMT Hemisuccinate | (formulated using TIP) | (see Part 4) |

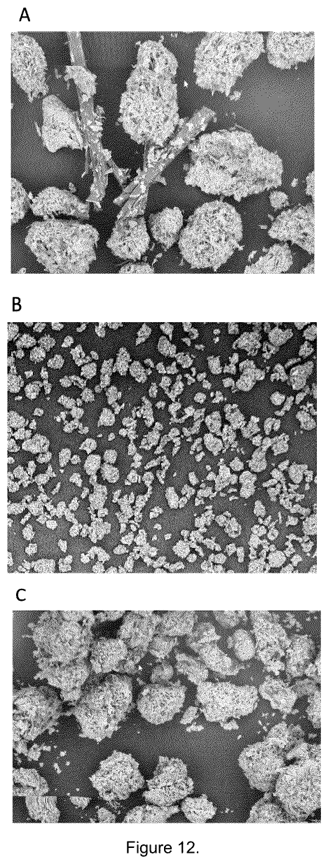

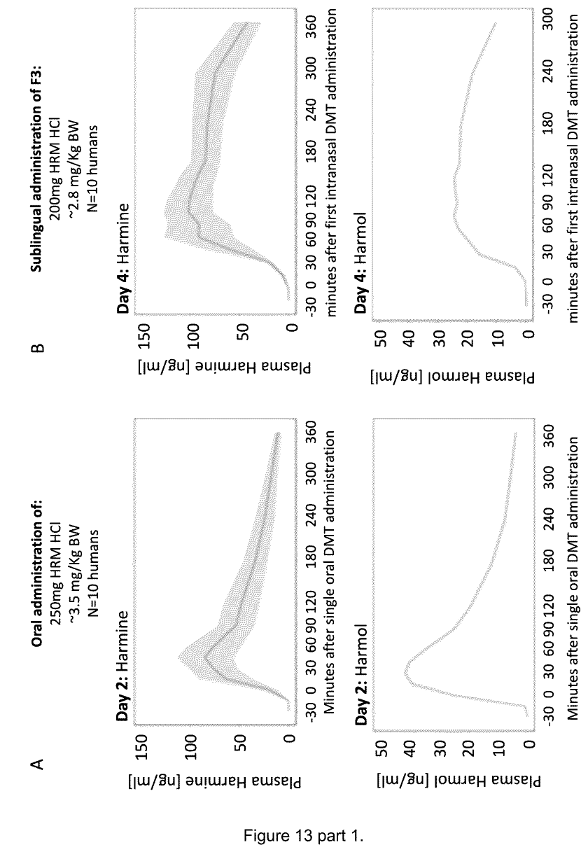

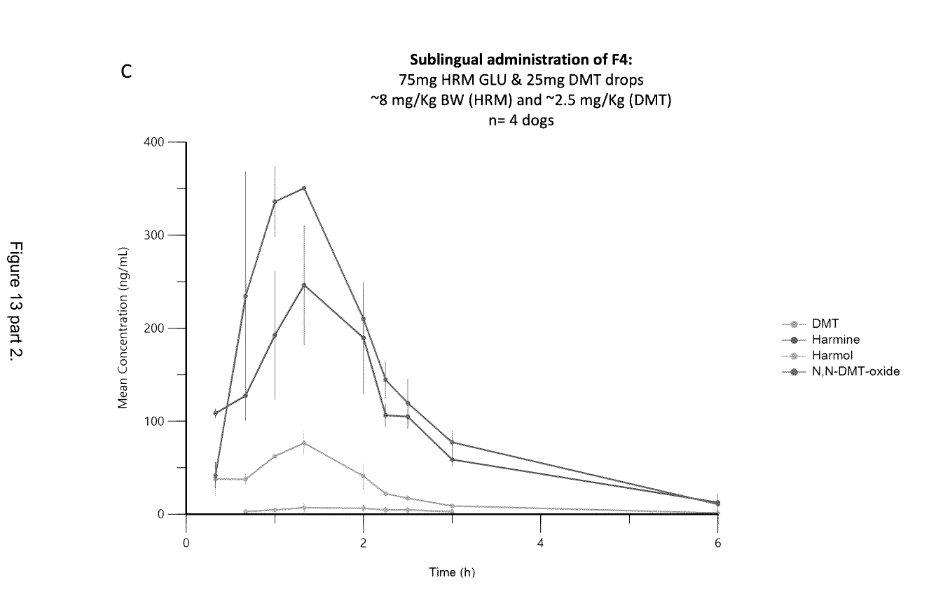

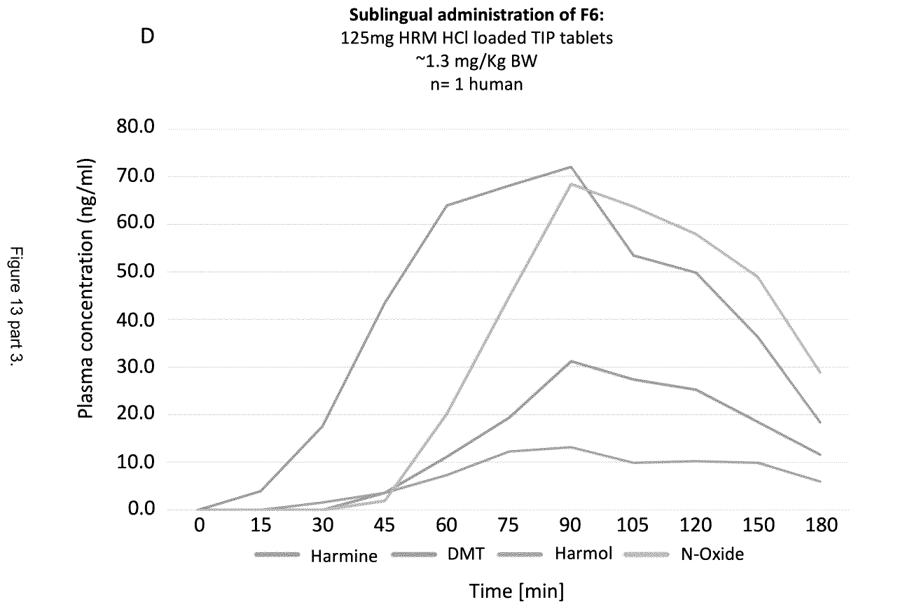

Formulation 1: Harmine freebase was milled and sieved (0.1 mm). A dose of 100 mg was placed between gingiva and lip of a volunteer to investigate mucosal absorption of the compound. After >1 h, the major part of the compound was still present in the buccal area, indicating a very poor ability of harmine to cross the mucosal membranes. Thus, this approach was not further investigated.
Formulation 2: Harmine hemifumarate was milled and sieved (0.1 mm). A dose of 125 mg (equiv. to 100 mg harmine freebase) was placed between gingiva and lip of a volunteer to investigate mucosal absorption of the compound. After >1 h, the major part of the compound was still present in the buccal area, indicating a very poor ability of harmine to cross the mucosal membranes. Thus, this approach was not further investigated.
Formulation 3: Harmine HCl (4000 mg) and mannitol (bulking agent; 4000 mg) were dissolved in 20 ml of water at 50° C. Then, the solution was quickly transferred to aluminum molds (0.5 ml per cavity (corresp. to 100 mg). Given the low stability of the solution at low temperatures (Harmine HCl crystal formation), the solution was shock-frosted at −80° C. and then transferred to the freeze-drying machine, which was precooled to −80° C. to prevent defrosting of the samples. The samples were then freeze-dried for 36 h. The melting tablets were then given to 10 healthy volunteers and blood plasma profiles were assessed. Thereby, a dose of 200 mg (2 melting tablets, equiv. to 170 mg harmine freebase) was placed between gingiva and lip of the volunteers to investigate mucosal absorption of the compound. In all volunteers, the melting tablet completely dissolved within 15-30 min. PK profiles are depicted in FIG. 13.
Formulation 4: A sublingual drop solution containing DMT hemisuccinate and harmine glucuronate were formulated by sequentially dissolving (first harmine, then DMT) both compounds in dH2O. The final solution contained 750 mg harmine glucuronate (equiv. to 375 mg harmine FB) and 165 mg DMT hemisuccinate (equiv. to 125 mg DMT freebase) in 2.5 ml of dH2O. The sublingual dose was administered to 4 beagle dogs (2 male, 2 female) by dispensing a volume (0.1 ml) of the sublingual formulation under the tongue (using a 0.1 ml Eppendorf pipette) and then holding the mouth closed for a minimum of 30 seconds to allow the formulation to be absorbed. A total of 5 doses were administered 20 minutes apart to achieve the total dose (t0, t20 min, t40 min, t60 min, t80 min).
Formulation 5: A fast disintegrating sublingual tablet was manufactured using TIP particles. Accordingly, harmine HCl was dissolved in EtOH (99.8%) to yield a concentration of 1% (m/v). The specific amount of TIP particles was calculated to yield a loading coefficient of the particles of 25%. Then, EtOH was slowly evaporated in the rotary evaporator over 4 h. The scanning electron microscopic pictures revealed substantial external crystallization of harmine HCl and thus unsuccessful loading of the particles, wherefore this batch was discarded without further evaluation (FIG. 12). It was assumed that loading failed due to oversaturation of the ethanolic solution and thus the formation of harmine HCl crystals, wherefore the experiment was replicated with reduced concentration (Formulation 6).
Formulation 6: A fast disintegrating sublingual tablet was manufactured using TIP particles. Therefore, harmine HCl was dissolved in EtOH (99.8%) to yield a concentration of 0.1% (m/v). The specific amount of TIP particles was calculated to yield a loading coefficient of the particles of 25%. Then, the ethanolic solution was added to the correct amount of TIP particles and EtOH was slowly evaporated in the rotary evaporator over 4 h. The scanning electron microscopic pictures revealed no external crystallization of harmine HCl and thus a successful loading of the particles (FIG. 12). The sublingual dose was administered to one healthy volunteer, by placing an amount of powder equivalent to 20 mg of harmine freebase under the tongue. A total of 5 doses were administered 20 minutes apart to achieve the total dose of 100 mg (t0, t20 min, t40 min, t60 min, t80 min).
Formulation 7: A fast disintegrating sublingual tablet was manufactured using TIP particles. Therefore, harmine glucuronate was dissolved in dH2O to yield a concentration of 25% (m/v). The specific amount of TIP particles was calculated to yield a loading coefficient of the particles of 25%. Then, the aqueous solution was slowly dropped onto the powder (in a Petri dish) and constantly stirred to yield a homogeneous paste. The paste was then air-dried at room temperature overnight. The scanning electron microscopic pictures revealed no external crystallization of harmine glucuronate and thus a successful loading of the particles (FIG. 12). Formulation 7 will be evaluated in the planned PKPD study described in detail below (Part 4).

Palatability. The bitterness of each formulation (F1-7) was examined in 5 volunteers using a “0-10 numeric bitterness rating scale” (0=none; 1-3=mild; 4-6=moderate, 7-10=severe).

Pharmacokinetics. Pharmacokinetic properties were investigated in humans and dogs. In more detail, F1 and F2 were tested on 3 healthy volunteers. Since both formulations were not absorbed from the mucosa (major parts of the administered dose still present in the buccal area after >1 h), PK profiles were not further investigated. F3 was tested in 10 healthy volunteers, F4 was tested in 4 beagle dogs (2 male, 2 female), F6 was tested in one healthy volunteer and F7 will be tested in 8 healthy volunteers (detailed description in Part 4). F5 was not further investigated in humans, given the unsuccessful manufacturing process (incomplete loading of Harmine HCl into the TIP carrier system) and inability to scale the process up to an industrial scale.

Blood Analysis for the human PK trials: DMT was purchased from Cayman (Ann Arbor, USA), harmine and NMT (N-ω-methyltryptamine) were purchased from Sigma-Aldrich (St. Louis, USA) and DMT-d6 were purchased from Toronto Research Chemicals (Toronto, Canada). All other used chemicals were of highest grade available. For the sample preparation 200 μl of plasma, 50 μl of the internal standard (IS) (20 ng/ml DMT-d6) and 50 μl of Methanol (MeOH) were added to a tube. Proteins were precipitated by adding 400 μl of acetonitrile (ACN) and samples were shaken for 10 minutes and centrifuged for 5 min at 10′000 rpm. 350 μl of the supernatant was further transferred into an auto-sampler vial, evaporated to dryness under a gentle stream of nitrogen and reconstituted with 250 μl of an eluent-mixture (98:2, v/v). Calibrator and quality control (QC) samples were prepared accordingly, replacing the MeOH with calibrator or QC solutions. Samples were analyzed on an ultra-high performance liquid chromatography (UHPLC) system (Thermo Fisher, San Jose, CA) coupled to a linear ion trap quadrupole mass spectrometer 5500 (Sciex, Darmstadt, Germany). The mobile phases consisted of a mixture of water (eluent A) and ACN (eluent B), both containing 0.1% of formic acid (v/v). Using a Kinetex C18 column (100×2.1 mm, 1.7 μm) (Phenomenex, Aschaffenburg, Germany), the flow rate was set to 0.5 mL/min with the following gradient: start conditions 98% of eluent A for 0.8 min, decreasing to 60% within 6.7 min followed by a quick decrease to 8% within 0.1 min. These conditions were held for 0.9 min and switched to the starting conditions for re-equilibration for 0.5 min. The mass spectrometer was operated in positive electrospray ionization mode with scheduled multiple reaction monitoring events. The following transitions of precursor ions to product ions were selected: DMT, m/z 189.1->58.2, DMT-D3, m/z, 195.1->64.1, harmine, m/z 213.0->169.2, NMT, m/z 175.1->144.0. The concentration range in calibration standards was 0.5 ng/ml to 60 ng/ml for DMT, 3 ng/ml to 360 ng/ml for harmine and 0.5 to 60 ng/ml for NMT. Thus, the lower limit of sensitivity was 0.5 mg/ml for DMT, 3 ng/ml for harmine and 0.5 ng/ml for NMT.

Blood Analysis for the human PK trials: Plasma samples were analysed using an established RGA2 LC-MS/MS assay. Harmine, DMT, harmol and DMT N-oxide were weighed and dissolved in acetonitrile/dimethyl sulfoxide (50/50, v/v). Harmine and DMT N-oxide stock solution were prepared in amber glass and DMT and harmol stock solutions were prepared in glass unprotected from light. Calibration standard and QC stock solutions were diluted in acetonitrile to give solutions of 200 μg/mL and further diluted in dog plasma (K₂EDTA) to give solutions which generate matrix concentrations 1.00-5000 ng/mL. Nifedipine was used as the internal standard for harmine, DMT, harmol and DMT N-oxide. This was dissolved in dimethyl sulfoxide, with further dilutions in acetonitrile and spiked at a solution concentration of 0.02 μg/mL. Calibration standard and QC dilutions were prepared in plastic, and internal standard solutions were prepared in amber glass.

### Plasma Sample Extraction

Control dog plasma (K₂EDTA), standard, QC, or test sample (50 μL) was aliquoted to the bottom of a 96 round well plate. A protein precipitation was performed by adding 150 μL of internal standard to calibration standards, QCs, single blanks and test samples (150 μL acetonitrile to double blanks). The plate was vortex mixed and centrifuged for 5 minutes, 2400×g at a temperature set to maintain 4° C. Using a Tomtec robot, 150 μL of deionised water/formic acid (100/0.2, v/v) was aliquoted to a clean 96 round well plate, followed by 75 L of supernatant. The plate was then vortex mixed briefly and centrifuged for 5 minutes, 2400×g at a temperature set to maintain 4° C. prior to analysis.

### Results

Loading harmine or its salt into TIP particles: FIG. 12 depicts SEM images of unsuccessful TIP loading with Harmine HCl (A; 1% in EtOH) and successful loadings with Harmine HCl (B; 0.1% in EtOH) and Harmine Glucuronate (C; 25% in H₂O). As shown, TIP particles can be loaded with Harmine HCl, but only when using very low substance concentrations (0.1%) in an organic solvent such as EtOH. Loading with higher concentrations (1%) leads to external crystallization. Moreover, using H₂O as solvent, triggers the rapid precipitation of HRM HCl as freebase, when it is exposed to basic residues within the TIP particles. Thus, given the incompatibility of H₂O as a solvent and given the poor solubility of HRM HCl in EtOH, loading solutions with a concentration of 0.1% in EtOH had to be used, which 1) is unpractical to handle in a large scale (e.g. 1 Kg of HRM HCl would have to be dissolved in 1000 L of EtOH, which is unpractical, expensive and poses the additional risk of using flammable/explosive solvents) and 2) eventually yields very poor loading results, given the very low HRM HCl concentration in the loading solution. Regarding the latter point, it could be shown that the loading process of the TIP particles is most efficient, when adding highly concentrated drug substance solutions (either in EtOH, H₂O or DMSO) to the particles. Loading TIP particles with harmine glucuronate yielded exceptional results. This is mainly due to the high solubility of Harmine Glucuronate in H₂O (˜45% at RT) and its ability to form stable solutions even when exposed to basic residues of the TIP particles. It is thought that this property of HRM GLU is caused by its highly amorphous structure, leading to less crystallization compared to HRM HCl (which crystallizes readily as freebase, especially when exposed to basic residues within the TIP particles).

Thus, harmine glucuronate is very practical to handle on a large scale (e.g. up to 1 Kg of HRM GLU can be dissolved in 1 L of water), yields very efficient loading of the TIP particles.

Palatability. 5 volunteers rated the bitterness of Formulations F1-F7 on a 0-10 numeric rating scale (0=none; 1-3=mild; 4-6=moderate, 7-10=severe). As depicted in Table 16, F3 and F4 were rated as severely bitter, whereas all other formulations were perceived as not or mildly bitter.

TABLE 16
Bitterness ratings of F1-F7 in 5 volunteers on a 0-10 numeric
rating scale (0 = none; 1-3 = mild; 4-6 =
moderate, 7-10 = severe). Mean values, standard deviations
(SD) and severity range are indicated.

| | F1 | F2 | F3 | F4 | F5 | F6 | F7 |
|---|---|---|---|---|---|---|---|
| Mean | 0.4 | 0.6 | 9.0 | 9.2 | 0.8 | 0.6 | 1.0 |
| SD | 0.5 | 0.8 | 1.1 | 0.7 | 0.7 | 0.5 | 0.6 |
| Sever- | none | none | severe | severe | none | none | none |
| ity | to | to | | | to | to | to |
| | mild | mild | | | mild | mild | mild |

Pharmacokinetic properties. In FIG. 12 blood plasma profiles of harmine and its hepatic metabolite harmol are shown, following the administration of A) HRM HCL as oral capsule (reference condition) in 10 healthy volunteers, B) F3, C) F4 and D) F6. As depicted on panels A and B, the bioavailability of harmine is increased by delivering it via the sublingual route, most likely due to more efficient sublingual absorption compared to gastrointestinal absorption and reduced first-pass metabolism. The reduction of first-pass metabolism following sublingual vs. oral administration becomes even more evident when comparing the ratio of harmine to its hepatic metabolite harmol, which is a direct measure for first-pass metabolism. Following the oral administration of HRM HCl (A) the ratio of harmine to harmol is approximately 2:1 (80 ng/ml:30 ng/ml). Following the sublingual administration of F3 (B) the ratio of harmine to harmol is increased to approximately 4:1 (100 ng/ml:25 ng/ml), indicating the circumvention of first-pass metabolism via the sublingual route. This effect becomes even more pronounced when using HRM GLU sublingual drops (F4) instead of HRM HCl sublingual ODTs (F3). In this case (F4), the harmine to harmol ratio is approximately 50:1 (250 ng/ml:5 ng/ml), which (without wishing to be bound by the theory) may be due to a highly efficient mucosal absorption of HRM GLU. The high solubility of HRM GLU may result in a high concentration gradient between the formulation and the mucosal tissue, leading to stronger absorption of HRM GLU across mucosal membranes. Moreover, its amorphous structure may also improve its ability to cross membranes. Similarly, HRM HCl loaded TIP particles (F6) reduced first-pass metabolism compared to oral or sublingual HRM HCl (F3) formulations, such that the harmine to harmol ratio is approximately 7:1 (70 ng/ml:10 ng/ml). The difference between the harmine to harmol ratio following administration of F4 and F6 may arise from the fact that, in experiment C, a much higher harmine doses were administered (8 vs. 1.3 mg/kg BW), which may have led to the saturation of harmine degrading enzyme systems and thus a disproportional increase of harmine levels compared to its metabolite harmol.

Beside the harmine to harmol ratio as an indicator of first-pass metabolism, the Cmax values also indicate the proportion of drug that has been absorbed into the blood system. Again, it can be seen that sublingual administration leads to improved bioavailability (due to better absorption and decreased first-pass metabolism), such that the oral administration of ˜3.5 mg/kg HRM HCl (A) yields mean Cmax values of ˜80 ng/ml, whereas sublingual administration of ˜2.8 mg/kg BW of F3 (B) leads to substantially higher mean Cmax value of ˜100 ng/ml. Similarly, the sublingual administration of ˜8 mg/kg BW of F4 leads to mean Cmax values of 250 ng/ml, even though the dose was not given as a bolus, but was split into 5 identical increments administered 20 minutes apart (t0, t20 min, t40 min, t60 min, t80 min). Thus, even higher Cmax values would have been expected, if the entire dose of F4 would have been administered as a bolus, further underlying the higher bioavailability of harmine following sublingual compared to oral administration. Interestingly, the sublingual administration of ˜1.3 mg/KG BW of F6, yields a Cmax of 70 ng/ml, even though the dose was not given as a bolus, but was split into 5 identical increments administered 20 minutes apart (t0, t20 min, t40 min, t60 min, t80 min). Thus, even higher Cmax values would have been expected, if the entire dose of F6 would have been administered as a bolus, further underlying the higher bioavailability of harmine following sublingual compared to oral administration.

### CONCLUSIONS

Taken together, sublingual delivery of harmine yields higher bioavailability and reduced first-pass metabolism compared to oral administration. This was the case for formulations F3, F4 and F6. Anyhow, F3 and F4 were rated as extremely bitter which substantially limits the use of these formulas. Moreover, severe bitterness (but also taste masking agents such as flavors or sweaters) trigger saliva production and increased swallowing, which again reduces the residence time of the compound in the oromucosal cavity and thus leads to dose loss via first-pass metabolism and increased PK variability. Thus, F6 represents the preferred formula, that shows high bioavailability, low first-pass metabolism, very good palatability and can be easily manufactured in a small but also larger scale (e.g. using high shear mixer). Based on the present data, it is assumed that F7 performs equally well as F6 or even better, given the high solubility and membrane crossing characteristics of harmine glucuronate compared to harmine HCl.
   Part 4: Planned PKPD Study with New Harmine Formulation (Open-Label, within-Subject, Dose-Response Study of DMT and Harmine in Healthy Subjects)

Participants and Study Design: N=8 healthy female and male subjects (25-45 y, BMI 18.5-30) with no current or previous history of neurological or psychiatric disorder and no first-degree relatives with history of Axis-I psychiatric disorder will be recruited by medical screening. In this open-label pilot study, acute subjective effects and blood samples following the administration of different ratios and escalating doses of DMT and harmine as a sublingual single preparation are measured. Additionally, on one test day participants will receive only DMT as a sublingual preparation. As this is a dose-finding study, participants will be given preferable dose ranges and can dis-/continue further dose administration within the indicated margins to enhance safety and tolerability.

Pharmacological intervention: A standardized and quality-controlled sublingual pharmahuasca formulation containing N,N-Dimethyltryptamine hemifumarate and harmine glucuronate will be prepared according to well-established pharmaceutical procedures described in Part 2 of this section (Formulation 7). The fast disintegrating sublingual tablet made using TIP particles will be manufactured by powder blending 1) harmine glucuronate loaded TIP particles with 2) N,N-DMT Hemifumarate loaded TIP particles to yield a final strength per dose of 20 mg DMT (calculated corresponding freebase amount) and 20 mg harmine (calculated corresponding freebase amount) or 5 mg DMT DMT (calculated corresponding freebase amount) and 5 mg harmine (calculated corresponding freebase amount) for the low-dose condition.

In more detail, harmine glucuronate loaded TIP particles will be manufactured as follows: Harmine glucuronate will be dissolved in dH₂O to yield a concentration of 25% (m/v). The specific amount of TIP particles will be calculated to yield a loading coefficient of the particles of 25%. Then, the aqueous solution will be slowly dropped onto the powder (in a Petri dish) and constantly stirred to yield a homogeneous paste. The paste will then be air-dried at room temperature overnight.

Similarly, the N,N-DMT Hemifumarate loaded TIP particles will be manufactured as follows: N,N-DMT Hemifumarate will be dissolved in EtOH (>99%) to yield a 10% solution. The ethanolic solution will be added to the correct amount of TIP particles and EtOH will be slowly evaporated in the rotary evaporator for 2 h, at 40° C., at 100 mbar, with 0.8 bar N2 flux.

On the study days, the tablets will be sublingually administered using by the participants under the supervision of an experimenter. The sublingual formulation is being administered with varying fixed bolus doses of DMT and harmine with up to three fixed increments of DMT and harmine in 20 minutes intervals. Specifically, we test 4 different dosing conditions with varying DMT:harmine ratios of 1:1, 1:0.5, 1:2, and 1:0 (DMT:harmine), thus a maximum dose of 100 mg DMT will be co-administered with 0/50/100/200 mg harmine. The dose ratios of 1:1, 1:0.5, 1:2 will be administered in randomized order, the DMT only condition will be tested on the last study day (Day 4).

Study procedures: On all study days, participants undergo blood sampling from the left antecubital vein on 12 timepoints, i.e. at baseline, and 20, 40, 60, 80, 100, 120, 180, 240, 300, 360, 420 min after administration for analysis of DMT and harmine concentrations in plasma. The venous catheter is connected to Heidelberger plastic tube extensions, to collect blood samples without disturbing the subjects during their psychedelic experience. The intravenous line is kept patent with a slow drip (10 ml/h) of heparinized saline (1000 IU heparin in 0.9 g NaCl/dL; HEPARIN Bichsel; Bichsel AG, 3800 Unterseen, Switzerland). Blood samples are immediately centrifuged for 10 minutes at 2000 RCF and plasma samples are kept frozen at −80° C. until assay.

The intensity of subjective effects (psychometrics of acute effects) is monitored throughout the period of drug action at baseline, 30, 60, 90, 120, 180, 240, and 360 min after administration. We will include a series of well-established computer-based psychometric tools including visual analogue scales for various drug effects and side effects, the Altered States of Consciousness Rating Scale (5D/11D-ASC), Visual Analogue Scale (VAS), Drug-induced Adverse Effects (AES). Other well-established questionnaires are used as secondary endpoints, such as the Challenging Experiences Questionnaire (CEQ), Positive and Negative Affect Schedule (PANAS), Emotional breakthrough inventory (EBI), Psy-Flex Questionnaire (Psy-Flex), Nature Relatedness Scale (NR6), Persisting Effects Questionnaire (PEQ), MINDSENS composite index (MS), Acceptance and Action Questionnaire II (AAQ), Gratitude Questionnaire (GQ-6), Affective Neuroscience Personality Scale (ANPS), Connectedness Questionnaire (C-SOW), Sussex-Oxford Compassion Scale (SOCS), Visual Self-Transcendence Scale (VST), Psychological Insight Scale (PIS-6), Psychological Insight Questionnaire (PIQ), and the Symptom Checklist (SCL-90-R).

The participants are screened for (serious) adverse effects throughout the experiment by the study physician, including questionnaire-based assessments (visual analogue scale: 1-10) of physical and mental discomfort, breathing difficulties, racing heartbeat, chest or abdominal pains, unpleasant body sensations/muscle pains, headache, nausea, vomiting, and fainting at baseline, 30, 60, 120, 240, and 480 min after drug administration. Vital signs (systolic/diastolic blood pressure, heart rate, body temperature, blood oxygenation level) are monitored throughout the study at baseline, 30, 60, 120, 240, and 360 min after drug administration. The same protocol is used for all study days.

### REFERENCES

Barbosa, P. C. R. et al., 2012. Health status of ayahuasca users. S. D. Brandt & T. Passie, eds. Drug Testing and Analysis, 4(7-8), pp. 601-609.
Barker, S. A., 2018. N, N-Dimethyltryptamine (DMT), an Endogenous Hallucinogen: Past, Present, and Future Research to Determine Its Role and Function. Frontiers in neuroscience, 12, pp. 139-17.
Callaway, J. C. et al., 1996. Quantitation of N,N-dimethyltryptamine and harmala alkaloids in human plasma after oral dosing with ayahuasca. Journal of analytical toxicology, 20(6), pp. 492-497.
Domínguez-Clavé, E. et al., 2016. Ayahuasca: Pharmacology, neuroscience and therapeutic potential. Brain research bulletin.
Frecska, E., Bokor, P. & Winkelman, M., 2016. The Therapeutic Potentials of Ayahuasca: Possible Effects against Various Diseases of Civilization. Frontiers in pharmacology, 7(e42421), pp. 35-17.
Nichols, D. E., 2016. Psychedelics. Pharmacological Reviews, 68(2), pp. 264-355. Osório, F. de L. et al., 2015. Antidepressant effects of a single dose of ayahuasca in patients with recurrent depression: a preliminary report, 37(1), pp. 13-20.
Palhano-Fontes, F. et al., 2018. Rapid antidepressant effects of the psychedelic ayahuasca in treatment-resistant depression: a randomized placebo-controlled trial. Psychological medicine, 7, pp. 1-9.
Riba, J. et al., 2003. Human pharmacology of ayahuasca: subjective and cardiovascular effects, monoamine metabolite excretion, and pharmacokinetics. The Journal of pharmacology and experimental therapeutics, 306(1), pp. 73-83.
Sanacora, G. et al., 2016. Balancing the Promise and Risks of Ketamine Treatment for Mood Disorders.
Santos, Dos, R. G. et al., 2016. Antidepressive and anxiolytic effects of ayahuasca: a systematic literature review of animal and human studies, 38(1), pp. 65-72.

### Part 5: Preparation of Crystal Forms of DMT Hemisuccinate

### DMT Hemisuccinate Form B:

### Preparation Using MEK (Methyl Ethyl Ketone)

DMT (41.3 mg) was dissolved in MEK (800 μL), then heated to 50° C. Dissolved succinic acid (12.95 mg) in water (300 μL) was added to DMT solution. The solution was cooled down to room temperature afterwards and put in the refrigerator for 12 h, —no solid was formed. The solution was then transferred to a Schlenk vial. The LSM was completely dried at 50° C. under vacuum. The residue was an off-white powder with a little oily residue.

### Preparation Using 2-Propanol

DMT (44.3 mg) was dissolved in 2-propanol (1000 μL), then heated to 50° C. Dissolved succinic acid (13.90 mg) in water (300 μL) was added to the DMT solution. The solution was cooled down to room temperature afterwards and put in the refrigerator for 12 h, —no solid was formed. The solution was then transferred to a Schlenk vial. The LSM was completely dried at 50° C. under vacuum. The residue was a white powder without oily residue.

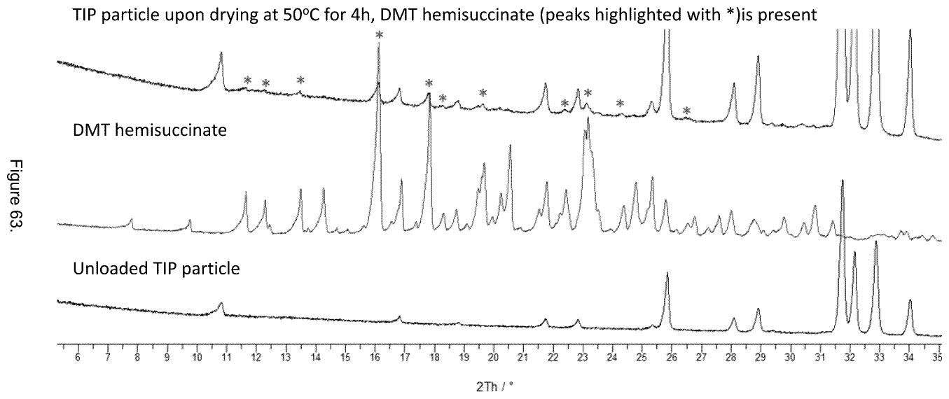

The identity of the compound has been confirmed using ¹H NMR measurements (see FIG. 62). Spectrum was acquired on a Bruker Advance DRX 400 spectrometer (at 400 MHZ) at room temperature in a deuterated solvent (d6-DMSO). Information about the chemical shift δ is given in ppm, relative to the irradiation frequency. The signal of the deuterated solvent is used as an internal standard.

### DMT Hemisuccinate Form A:

Variant with MEK (Methyl Ethyl Ketone)

Approx. 2 g DMT (0.5) were transferred into a 100 mL Schlenk flask and filled up to approx. 20 mL with water. ˜0.5 M NaOH solution was added until pH10 was reached and stable (min. 15 mL). Here the free base precipitated which subsequently became oil-like. The product was extracted with 80 mL MTBE and the solvent was completely removed on the rotary evaporator. The resulting oil was taken up in n-heptane (5 mL) and recrystallized. The crystals were filtered off and dried. Yield 1.34 g DMT

DMT (1 g) was dissolved in MEK (25 mL), then heated to 50° C. Dissolved succinic acid (0.5 equimolar) in water (5 mL) was added to the DMT solution. 20 mL were distilled off and the remaining solution was kept in the refrigerator for 3 h. As there was no crystallization, the solvent was largely evaporated open within 10 days. The molasses-like residue crystallized within another 4 days. The product has a slight yellow color. The product was DMT hemisuccinate Form A with traces of DMTS.

For Powder X-Ray Diffraction (XRPD) the samples were placed into standard glass capillaries (Ø=0.7 mm). The measurements were performed at room temperature with a D8 Bruker Advance Diffractometer (Cu-Kα₁=1.54059 Å, Johansson primary beam monochromator, position-sensitive detector) in transmission mode with the rotation of the sample. Data were collected in a two-theta range of 2-40°. The tube voltage and current were 40 kV and 40 mA, respectively. The measurements were performed for both form A as well as form B. The peak tables are shown in the following.

Stoichiometry: DMT Hemisuccinate
Form: Form B

| Angle | | Rel. |
|---|---|---|
| (° 2theta) | d-value | Intensity |
| 6.588 | 13.40511 | 4.10% |
| 7.718 | 11.44571 | 1.00% |
| 10.09 | 8.75983 | 20.10% |
| 11.05 | 8.00071 | 1.10% |
| 12.45 | 7.10375 | 2.80% |
| 13.266 | 6.66871 | 2.40% |
| 14.271 | 6.20121 | 1.20% |
| 15.57 | 5.6866 | 100.00% |
| 16.526 | 5.35988 | 43.40% |
| 16.816 | 5.26794 | 38.00% |
| 17.059 | 5.19358 | 24.90% |
| 17.501 | 5.06346 | 2.20% |
| 18.097 | 4.89784 | 2.60% |
| 18.408 | 4.81597 | 5.30% |
| 19.336 | 4.58689 | 27.70% |
| 19.584 | 4.52924 | 17.10% |
| 19.925 | 4.45247 | 20.40% |
| 21.133 | 4.20073 | 25.90% |
| 21.388 | 4.15105 | 15.40% |
| 22.068 | 4.02475 | 13.10% |
| 22.908 | 3.87908 | 72.90% |
| 23.45 | 3.7905 | 62.60% |
| 23.807 | 3.73461 | 10.20% |
| 24.14 | 3.6838 | 16.50% |
| 24.576 | 3.61936 | 2.40% |
| 25.432 | 3.49948 | 7.50% |
| 25.821 | 3.44768 | 4.60% |
| 27.226 | 3.27281 | 5.60% |
| 27.834 | 3.20268 | 8.70% |
| 28.598 | 3.11883 | 10.10% |
| 28.737 | 3.10411 | 11.10% |
| 29.869 | 2.98895 | 2.40% |
| 30.098 | 2.96676 | 3.80% |
| 30.771 | 2.90335 | 4.80% |
| 31.461 | 2.84128 | 2.00% |
| 31.779 | 2.81354 | 1.50% |
| 32.231 | 2.77515 | 1.10% |
| 32.628 | 2.74225 | 1.60% |
| 32.861 | 2.72337 | 1.20% |
| 33.57 | 2.66744 | 2.00% |
| 33.718 | 2.65604 | 2.90% |
| 34.24 | 2.61674 | 4.80% |
| 34.715 | 2.58201 | 2.00% |
| 35.216 | 2.54643 | 2.20% |
| 36.345 | 2.46985 | 2.70% |
| 36.917 | 2.4329 | 1.60% |
| 37.587 | 2.39103 | 1.20% |
| 38.17 | 2.35586 | 0.90% |
| 38.61 | 2.33006 | 1.40% |
| 39.571 | 2.27562 | 0.80% |
| 39.935 | 2.25573 | 2.80% |
| 40.276 | 2.2374 | 0.80% |
| 40.662 | 2.21706 | 0.50% |
| 41.343 | 2.18211 | 1.50% |
| 41.643 | 2.16706 | 1.70% |
| 41.883 | 2.15518 | 3.10% |
| 42.135 | 2.14291 | 1.70% |
| 43.327 | 2.08667 | 0.70% |
| 44.137 | 2.05024 | 0.50% |
| 45.035 | 2.01141 | 0.50% |
| 45.447 | 1.99414 | 0.80% |
| 46.862 | 1.93718 | 1.40% |
| 47.82 | 1.90057 | 0.50% |
| 48.71 | 1.86791 | 1.20% |
| 49.23 | 1.84936 | 1.10% |

Stoichiometry: DMT Hemisuccinate
Form: Form A

| Angle | | Rel. |
|---|---|---|
| (° 2theta) | d-value | Intensity |
| 7.813 | 11.3059 | 5.60% |
| 11.659 | 7.58426 | 18.60% |
| 12.309 | 7.18515 | 16.50% |
| 12.464 | 7.09589 | 4.00% |
| 13.498 | 6.55476 | 22.70% |
| 13.754 | 6.43298 | 2.20% |
| 14.327 | 6.17712 | 7.90% |
| 15.064 | 5.87663 | 1.70% |
| 16.143 | 5.48609 | 100.00% |
| 16.53 | 5.3585 | 2.10% |
| 16.733 | 5.29414 | 2.00% |
| 17.837 | 4.96881 | 71.80% |
| 18.334 | 4.8352 | 9.80% |
| 18.766 | 4.72483 | 10.50% |
| 19.082 | 4.64724 | 3.50% |
| 19.478 | 4.55371 | 16.30% |
| 19.674 | 4.5087 | 35.10% |
| 19.986 | 4.43909 | 8.40% |
| 20.248 | 4.38213 | 18.00% |
| 21.541 | 4.12208 | 9.30% |
| 21.813 | 4.07128 | 29.30% |
| 22.451 | 3.95699 | 16.70% |
| 23.19 | 3.83241 | 51.50% |
| 23.565 | 3.77227 | 11.90% |
| 23.95 | 3.71254 | 1.50% |
| 24.377 | 3.64852 | 12.80% |
| 24.83 | 3.58293 | 8.00% |
| 25.063 | 3.5502 | 4.90% |
| 25.356 | 3.50985 | 28.20% |
| 25.79 | 3.45171 | 15.30% |
| 26.537 | 3.3562 | 6.60% |
| 26.721 | 3.33357 | 2.20% |
| 27.167 | 3.27977 | 1.60% |
| 28.016 | 3.18231 | 13.00% |
| 28.705 | 3.10751 | 4.00% |
| 28.865 | 3.09065 | 5.90% |
| 29.417 | 3.03386 | 3.10% |
| 29.789 | 2.99683 | 10.70% |
| 30.004 | 2.97584 | 1.70% |
| 30.479 | 2.9305 | 8.20% |
| 30.823 | 2.89863 | 10.90% |
| 31.054 | 2.87752 | 1.60% |
| 31.344 | 2.85155 | 1.40% |
| 31.702 | 2.82019 | 1.70% |
| 32.032 | 2.79191 | 1.20% |
| 32.947 | 2.71641 | 1.30% |
| 33.098 | 2.70435 | 2.00% |
| 33.394 | 2.68105 | 3.10% |
| 33.908 | 2.6416 | 2.90% |
| 34.296 | 2.61257 | 1.30% |
| 34.451 | 2.6012 | 1.10% |
| 34.772 | 2.57789 | 2.10% |
| 35.328 | 2.53862 | 0.90% |
| 35.787 | 2.50707 | 2.40% |
| 36.763 | 2.44277 | 1.10% |
| 36.989 | 2.42832 | 2.10% |
| 37.819 | 2.3769 | 4.30% |
| 38.286 | 2.34902 | 5.10% |
| 38.498 | 2.33657 | 3.70% |
| 38.788 | 2.31974 | 0.60% |
| 39.394 | 2.28545 | 0.80% |
| 39.667 | 2.27032 | 1.20% |
| 39.999 | 2.25228 | 2.70% |
| 40.378 | 2.23197 | 1.60% |
| 40.886 | 2.20545 | 1.40% |
| 41.127 | 2.19305 | 2.10% |
| 41.471 | 2.17567 | 0.80% |
| 41.931 | 2.15283 | 1.60% |
| 42.177 | 2.14086 | 1.20% |
| 42.32 | 2.13394 | 0.80% |
| 43.419 | 2.08246 | 0.60% |
| 43.9 | 2.06073 | 1.50% |
| 44.583 | 2.03073 | 1.30% |
| 44.931 | 2.01581 | 1.40% |
| 45.288 | 2.00077 | 0.80% |
| 45.698 | 1.98376 | 1.80% |
| 46.556 | 1.94916 | 0.40% |
| 47.651 | 1.90689 | 1.10% |
| 47.918 | 1.89692 | 1.40% |
| 48.288 | 1.88324 | 1.80% |
| 48.557 | 1.87344 | 0.90% |
| 49.13 | 1.8529 | 0.90% |
| 49.397 | 1.84352 | 0.70% |
| 49.594 | 1.83664 | 0.70% |

### Part 6: Manufacturing of TIP-Based ODT Containing Different Ratios of Harmine Glucuronate and DMT Hemisuccinate

### Harmine Glucuronate Loading on TIP (25% HRM-GLC)

| Ingredients | Mass [g] |
|---|---|
| Harmine FB (free Base) | 17.640 |
| D-Glucuronic acid | 16.941 |
| TIP | 103.744 |
| Aqua Purificata | 120 |
| Total after drying | 138.326 |

### Procedure

### A: Manufacturing of Harmine Glucuronate Solution (HRM-Glc-SOL)

Steps

| | |
|---|---|
| 1) | Weigh glucuronic acid and fill into a 200 ml erlenmeyer flask |
| 2) | Add water and stir on the magnetic plate |
| 3) | Add harmine freebase and stir at room temperature until fully |
| | transparent solution is obtained. |

### B: Loading of Harmine Glucuronate on TIP

Steps

| | |
|---|---|
| 1) | TIP is weighed out and placed in a crystallizing dish (500 ml). |
| 2) | HRM-Glc-Sol is dispensed dropwise onto the TIP using a |
| | 10 ml syringe. Approximately 30 drops are added |
| | (approximately 1 drop per 2 seconds). Care is taken that the |
| | drop falls on the powder and does not land on the vessel |
| | wall. |
| 3) | The powder is then mixed with a spatula until a |
| | homogeneous powder mixture is obtained. Any lumps are |
| | reduced by means of the spatula. |
| 4) | Step 2) is repeated, until ¼ of the entire volume (ca. 35 ml) |
| | has been added. |
| 5) | After that the powder mixture is dried in the oven for 2 h at |
| | 40° C. |
| 6) | Step 2) is repeated until half of the solution volume (70 ml) |
| | has been added. |
| 7) | After that, the powder mixture is intermediately dried in the |
| | oven at 40° C. for 2 h. |
| 8) | Step 2) is repeated until about ¾ of the total volume has |
| | been added. |
| 9) | After that, the powder mixture is intermediately dried in the |
| | oven at 40° C. for 2 h. |
| 10) | Step 2) is repeated until the total volume has been added. |
| 11) | Then, the completely loaded powder mixture is dried in the |
| | oven at 40° C. until the weight constant is reached (approx. |
| | 8 h). |

### DMT Hemisuccinate Loading on TIP

| Ingredients | Mass [g] |
|---|---|
| DMT Succinate | 22.650 |
| NaOH | 2.957 |
| TIP | 72.828 |
| EtOH 96% | 60 ml |
| Aqua Purificata | 80 ml |
| Total after drying | 97.105 |

### Procedure

### A: Manufacturing of DMT-Monosodiumsuccinate-Solution

Steps

| | |
|---|---|
| 1) | NaOH is weighed and dissolved in 12.5 ml H2O. |
| 2) | DMT Succinate is weighed and suspended in 60 ml EtOH. |
| 3) | Add the NaOH-Lsg dropwise to the DMT Succinate |
| | suspension by syringe while stirring and stir at RT |
| | until a clear solution is obtained. |

### B: Loading of DMT-Monosodiumsuccinate-Solution on TIP

Steps

| | |
|---|---|
| 4) | TIP is weighed out and placed in a crystallizing dish (500 ml). |
| 5) | DMT-monosodium succinate solution is dispensed dropwise |
| | onto the TIP using a 10 ml syringe. Approximately 30 drops |
| | are added (approximately 1 drop per 2 seconds). Care is |
| | taken that the drop falls on the powder and does not land on |
| | the vessel wall. |
| 6) | The powder is then mixed with a spatula until a |
| | homogeneous powder mixture is obtained. Any lumps are |
| | reduced by means of the spatula. |
| 7) | Step 2) is repeated until ¼ of the total volume (approx. 35 ml) |
| | has been added. |
| 8) | After that, the powder mixture is intermediately dried in the |
| | oven at 40° C. for 2 h. |
| 9) | Step 2) is repeated until half of the total volume (approx. |
| | 70 ml) has been added. |
| 10) | After that, the powder mixture is intermediately dried in the |
| | oven at 40° C. for 2 h. |
| 11) | Step 2) is repeated until ¾ of the total volume has been |
| | added. |
| 12) | After that, the powder mixture is intermediately dried in the |
| | oven at 40° C. for 2 h. |
| 13) | Step 2) is repeated until the total volume has been added. |
| 14) | Then, the completely loaded powder mixture is dried in the |
| | oven at 40° C. until the weight constant is reached (approx. |
| | 8 h). |

XRPD measurements (performed as described in part 5) revealed the formation of DMT Hemisuccinate in the TIP particles, as discussed in the following particular example.

Loading of TIP (Template Inverted Particle/Ca₅[(OH)(PO₄)₃])
DMTS Loading [20%], Neutralized with 1 Eq. NaOH

### Preparation DMT Succinate Solution (Neutralized):

202.4 mg DMT succinate was placed in a beaker. 0.4 mL water and 0.2 mL ethanol were added. The mixture was heated slightly (40° C.) and stirred until a clear solution was obtained. NaOH (1N solution) was added very slowly (to prevent precipitation of DMT) and stirred until a clear solution was formed. The pH was about 7. The entire solution was drawn up into a 2 ml syringe.

### Loading TIP Particles:

808.4 mg TIP was weighed out and placed in a crystallizing dish (the smaller the better—this ensures that the solution falls onto the powder and not onto the walls or bottom of the vessel). The crystallizing dish was now heated to 40° C. on a hot plate. Now, about 0.25 ml of DMTS solution was slowly spread directly onto the powder. Using a spatula, the liquid was distributed homogeneously over the entire powder by stirring and reducing any lumps. If the powder already appeared very moist, the powder was dried briefly (40° C.+convection). The dripping/intermediate drying procedure was repeated until the entire volume was added. The dripping could be faster in the beginning and was slowed down towards the end (when most of the particles are already loaded) to prevent over-wetting of the powder (and thus the risk of external crystallization). The final product was dried in several steps. The XRPD analysis has revealed the presence of DMT hemisuccinate in the TIP particles (see FIG. 63).

### Manufacturing ODT Tablets:

The amount of 1) harmine glucuronate loaded TIP, DMT Hemisuccinate loaded TIP, 3) unloaded TIP, 4) Ac-Di-Sol, 5) menthol, 6) sucralose and 7) peppermint flavor as given in the table below are blended using a turbula powder mixer (10 min). Then, each tablet (15 mm) is manually compressed using a table top tablet press.

| | DMT- | | | | | | | Total |
|---|---|---|---|---|---|---|---|---|
| HAR-GIC- | Hemisuccinate- | unloaded | Ac-Di- | | | Peppermint | Total | weight per |
| TIP-25% | TIP-25% | TIP | Sol | Menthol | Sucralose | Flavour | weight | tablet |
| [g] | [g] | [g] 5% | [g] 3% | [g] | [g] | [g] | [g] | [g] |
| 31.05 | 13.81 | 2.40 | 1.48 | 0.22 | 0.22 | 0.22 | 49.42 | 0.75 |

Part 7: Single-Blind, Randomized, Two-Arm, Dose-Response Study of DMT and Harmine in Healthy Subjects (Open-Label, within-Subject, Dose-Response Study of DMT and Harmine in Healthy Subjects)

Participants and Study Design: 12 healthy female and male subjects (25-45 y) with no current or previous history of neurological or psychiatric disorder and no first-degree relatives with history of Axis-I psychiatric disorder were recruited by medical screening. In this single-blind pilot study, acute subjective effects and blood samples following the administration of escalating doses of DMT and harmine as a sublingual single preparation were measured. Additionally, on the fourth test day participants received either DMT or harmine only as a sublingual preparation according to their arm allocation. Study participants completed a telephone and medical screening before enrolment to the study. The study was approved by the Cantonal Ethics Committee of the Canton of Zurich (Basec-Nr. 2022-00973) and Swiss Federal Office of Public Health (BAG-Nr. (AB)-8/5-BetmG-2022/018086). All participants provided written informed consent according to the declaration of Helsinki and were monetary compensated for the completion of the study.

Study setting: The study was conducted during the daytime in a furnished group treatment room to provide a comfortable living room atmosphere with dimmable lights and sound systems. Throughout all study days, a standardized playlist containing non-stimulating background music was played to provide a feeling of comfort and relaxation, with silence periods in between. Up to 4 participants were co-administered with the substance on a study day with experimenters present in the room all the time for supervision.

Pharmacological intervention: A standardized and quality-controlled sublingual formulation containing N,N-Dimethyltryptamine hemisuccinate and harmine glucuronate was prepared according to well-established pharmaceutical procedures described in Part 2 of this section (Formulation 7). The sublingual formulation was manufactured by blending the drugs with the GRAS (Generally Recognized As Safe) excipient calcium phosphate to form a homogenous powder blend. Moreover, sucralose (sweetener) and orange, menthol or peppermint flavor (aroma) was added for taste masking. The final formulation was compacted into fast disintegrating tablets made using TIP particles by powder blending 1) harmine glucuronate loaded TIP particles with 2) N,N-DMT Hemisuccinate loaded TIP particles to yield a final strength per dose of 0-60 mg harmine (corresp. to the freebase) and 0-40 mg DMT (corresp. to the freebase), administered at 3 dosing intervals every 20 minutes.

In more detail, harmine glucuronate loaded TIP particles was manufactured as follows: Harmine glucuronate was dissolved in dH2O to yield a concentration of 25% (m/v). The specific amount of TIP particles was calculated to yield a loading coefficient of the particles of 25%. Then, the aqueous solution was slowly dropped onto the powder (in a Petri dish) and constantly stirred to yield a homogeneous paste. The paste was then air-dried at room temperature overnight.

Similarly, the N,N-DMT Hemisuccinate loaded TIP particles were manufactured as follows: N,N-DMT Hemisuccinate was dissolved in EtOH (>99%) to yield a 10% solution. The ethanolic solution was added to the correct amount of TIP particles and EtOH was slowly evaporated in the rotary evaporator for 2 h, at 40° C., at 100 mbar, with 0.8 bar N2 flux.

On the study days, the tablets were sublingually administered by the participants on empty stomach (last meal >10 hours; last drink >90 mins) under the supervision of an experimenter. The sublingual formulation was administered with varying fixed bolus doses of DMT and harmine with two fixed increments of DMT and harmine in 20 minute intervals, resulting in 3 administrations over 40 minutes. Specifically, we tested 7 different dosing conditions with varying DMT:harmine ratios in the dose range between 0-120 mg DMT and 0-180 mg harmine. The dose ratios of harmine to DMT ranged from 0 to 2 (w/w) and were administered in a single-blind, within-subject, sequential ascending order with two different sequences in two arms.

The intensity of subjective effects (psychometrics of acute effects) was monitored at baseline, 0, 20, 40, 60, 85, 120, 150, 180, 240, 300, 540, 1440 (=24 h) after administration. We included a series of well-established computer-based psychometric tools including visual analogue scales (VAS) for various drug effects and side effects, the Altered States of Consciousness Rating Scale (5D/11D-ASC), acute subjective drug effects (VAS), and drug-induced adverse effects (AES). Other well-established questionnaires were used as secondary endpoints, such as the Mystical Experience Questionnaire (MEQ), Challenging Experiences Questionnaire (CEQ), Emotional breakthrough inventory (EBI), Psy-Flex Questionnaire (Psy-Flex), Persisting Effects Questionnaire (PEQ), MINDSENS composite index (MS), Gratitude Questionnaire (GQ-6), Affective Neuroscience Personality Scale (ANPS), Watts Connectedness Questionnaire (WCS), Sussex-Oxford Compassion Scale (SOCS), Visual Self-Transcendence Scale (VST), Psychological Insight Scale (PIS-6), WHO-5 Well-Being Index (WHO-5), Perceived Stress Scale (PSS), Griffiths Significance Ratings (GSR), Brief Symptom Checklist (BSCL), User Experience Questionnaire (USX), Sleep Quality Scale-Short (SQS-S).

The participants were screened for (serious) adverse effects throughout the experiment by the study physician, including questionnaire-based assessments (visual analogue scale: 1-10) at baseline, 0, 20, 40, 60, 85, 120, 150, 180, 240, and 360 min after drug administration. The following side-effect items were assessed: bodily symptoms/discomfort (breathing difficulties, heart racing, chest pain, stomach pain, unpleasant body sensations/muscle pain, headache, nausea, vomiting, fainting, psychological symptoms/discomfort (unspecific discomfort, anxiety, panic, delusions, agitation, dissociation, reduction of vigilance). Vital signs (systolic/diastolic blood pressure, heart rate, blood oxygenation level) were monitored throughout the study at baseline, 0, 20, 40, 60, 85, 120, 150, 180, 240, 300, 540, 1440 (=24 h) after drug administration. ECG was measured at baseline, 75, 150, 300, 540, and 1440 min after administration. Body temperature was measured at baseline, 75, 180, 300, 540, and 1440 min after administration. The same protocol was used for all study days. Participants were released at the end of the study day but will come back the following day for their assessments 24 hours after first substance administration.

On all study days, blood samples were taken from the left antecubital vein on 15 timepoints i.e. at baseline, and 0, 20, 40, 60, 70, 85, 100, 120, 150, 180, 240, 300, 420, 540, 1440 min after administration for analysis of DMT and harmine concentrations in plasma. The venous catheter was connected to Heidelberger plastic tube extensions, to collect blood samples without disturbing the subjects during their psychedelic experience. The intravenous line was kept patent with a slow drip (10 ml/h) of heparinized saline (1000 IU heparin in 0.9 g NaCl/dL; HEPARIN Bichsel; Bichsel AG, 3800 Unterseen, Switzerland). Blood samples were immediately centrifuged for 10 minutes at 2000 RCF and plasma samples were kept frozen at −80° C. until assay. DMT was purchased from Lipomed (Arlesheim, Switzerland), NMT and 3-IAA were purchased from Sigma-Aldrich (St. Louis, USA), and harmine, harmol, DMT-N-oxide, harmine-d3 and DMT-d6 were purchased from Toronto Research Chemicals (Toronto, Canada). All other used chemicals were of highest grade available. For the sample preparation 200 μl of plasma were spiked with 50 μl internal standard (IS) mixture (40 ng/ml DMT-d6 and harmine-d3) and 50 μl methanol (MeOH). Proteins were precipitated by adding 400 μl of acetonitrile (ACN). The samples were shaken for 10 minutes and centrifuged for 5 min at 10′000 rpm. 350 μl of the supernatant was transferred into an auto-sampler vial, evaporated to dryness under a gentle stream of nitrogen at room temperature and reconstituted in 100 μl eluent-mixture (98:2, v/v). External calibrator and quality control (QC) samples were prepared accordingly, replacing the MeOH with calibrator or QC solution mixtures. Calibrator and QC samples containing 3-IAA were prepared separately, replacing plasma by water. The calibration ranges were 0.5-500 ng/ml for DMT and DMT-N-oxide, 2.5-120 ng/ml for harmine, 1-80 ng/ml for harmol, 0.015-10 ng/ml for NMT and 35-3000 ng/ml for 3-IAA. Samples were analysed on an ultra-high performance liquid chromatography (UHPLC) system (Thermo Fisher, San Jose, CA) coupled to a linear ion trap quadrupole mass spectrometer 5500 (Sciex, Darmstadt, Germany). The mobile phases consisted of a mixture of water (eluent A) and ACN (eluent B), both containing 0.1% formic acid (v/v). Using a Kinetex C18 column 50×2.1 mm, 2.6 μm (Phenomenex, Aschaffenburg, Germany), the flow rate was set to 0.5 ml/min with the following gradient: starting conditions 98% eluent A, decreasing to 70% within 4 min, followed by a quick decrease to 5% within 1 min, holding for 0.5 min and returning to starting conditions for 1.5 min, resulting in a total runtime of 7 min. The mass spectrometer was operated in positive electrospray ionization mode with scheduled multiple reaction monitoring. The following transitions of precursor ions to product ions were selected as quantifier ions: DMT m/z 189→115, DMT-N-oxide m/z 205→117, harmine m/z 213→169, harmol m/z 199→131, NMT m/z 175→144 and 3-IAA m/z 176→103.

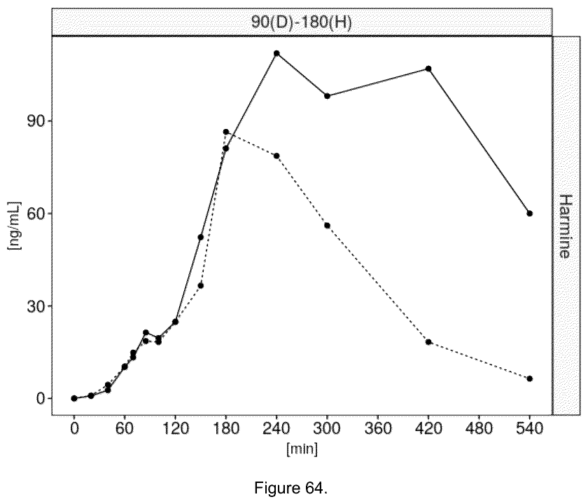

PK profiles were quantified from two representative subjects that received a high dose (180 mg) of harmine glucuronate together with a fixed dose of 90 mg DMT formulated in TIP as part of the previously described study (Part 7; actual PKPD study). As explained above, the ODTs were administered sublingually in 3 fixed dosing intervals every 20 minutes on empty stomach (last meal >10 hours; last drink >90 mins) under the supervision of an experimenter. On both study days, blood samples were taken from the left antecubital vein on 15 timepoints i.e. at baseline, and 0, 20, 40, 60, 70, 85, 100, 120, 150, 180, 240, 300, 420, 540, 1440 min after administration for analysis of DMT and harmine concentrations in plasma. The venous catheter was connected to Heidelberger plastic tube extensions, to collect blood samples without disturbing the subjects during their psychedelic experience. The intravenous line was kept patent with a slow drip (10 ml/h) of heparinized saline (1000 IU heparin in 0.9 g NaCl/dL; HEPARIN Bichsel; Bichsel AG, 3800 Unterseen, Switzerland). Blood samples were immediately centrifuged for 10 minutes at 2000 RCF and plasma samples were kept frozen at −80° C. until assay. The quantification of harmine in blood plasma was performed according to the methods described in the previous paragraph. The result is shown in FIG. 64. The experiment demonstrates that the so formulated glucuronate salt of harmine is bioavailable.

### Part 8: Bitterness of Harmine Glucuronate Formulations

Harmine HCL was loaded into TIP particles at 30% drug loading with ethanol in a rotary evaporation process. After removal of 90% of the initially used solvent, trace amount of water was added to the rotary evaporator and subsequently dried. Water was added to initiate the ionization of the Harmine HCL and Calcium hydroxide on the surface of the loaded TIP particles.

A physical mixture of TIP and Harmine HCL was used to compare the taste of the masked preparation. The physical mixture had aversive bitterness due to Harmine HCL. The TIP preparation was tasteless.

Harmine Glucoronate was loaded into TIP particles at 25% drug loading with water using capillary sorption process.

A physical mixture of TIP and Harmine Glucoronate was used to compare the taste of the masked preparation. The physical mixture had aversive bitterness due to Harmine Glucoronate. The TIP preparation was significantly less bitter.

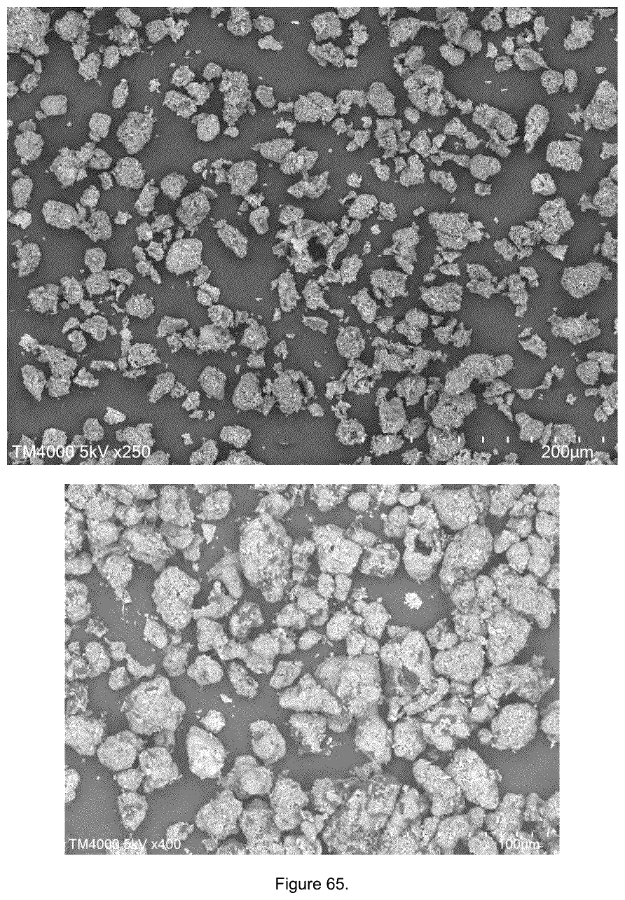

The loaded TIP particles are shown in FIG. 65.

The bitterness of 6 different harmine formulations was examined in 5 volunteers using a “0-10 numeric bitterness rating scale” (0=none; 1-3=mild; 4-6=moderate, 7-10=severe). Therefore, the amount of formulation corresponding to 20 mg harmine freebase was administered to the sublingual area of the volunteers. Manufacturing procedures of formulations 1 to 6 are presented below. An overview of the tested formulations and the results of the compound tasting are presented in table below:

| Salt | Harmine | Harmine | | Harmine | | Harmine |
|---|---|---|---|---|---|---|
| form | freebase | hemifumarate | Harmine HCl | glucuronate | Harmine HCl | glucuronate |
| Dosage | Pure API | Pure API | Freeze-dried | Aqueous drop | Fast | Fast |
| form | | | ODT | solution | Disintegrating | Disintegrating |
| | | | | | Tablet | Tablet |
| | | | | | (formulated | (formulated |
| | | | | | using TIP) | using TIP) |
| Mean | 0.4 | 0.6 | 9.0 | 9.2 | 0.6 | 1.0 |
| SD | 0.5 | 0.8 | 1.1 | 0.7 | 0.5 | 0.6 |
| Severity | none to mild | none to mild | severe | severe | none to mild | none to mild |

### Manufacturing Procedures for the Tested Formulations

Formulation 1: Harmine freebase was milled and sieved (0.1 mm).
Formulation 2: Harmine hemifumarate was milled and sieved (0.1 mm).
Formulation 3: Harmine HCl (4000 mg) and mannitol (bulking agent; 4000 mg) were dissolved in 20 ml of water at 50° C. Then, the solution was quickly transferred to aluminum molds (0.5 ml per cavity (corresp. to 100 mg). Given the low stability of the solution at low temperatures (Harmine HCl crystal formation), the solution was shock-frosted at −80° C. and then transferred to the freeze-drying machine, which was precooled to −80° C. to prevent defrosting of the samples.
Formulation 4: A sublingual drop solution containing harmine glucuronate was formulated dissolving the compound in dH2O. The final solution contained 750 mg harmine glucuronate (equiv. to 375 mg harmine FB) in 2.5 ml of dH2O.
Formulation 7: A fast disintegrating sublingual tablet was manufactured using TIP particles. Therefore, harmine HCl was dissolved in EtOH (99.8%) to yield a concentration of 0.1% (m/v). The specific amount of TIP particles was calculated to yield a loading coefficient of the particles of 25%. Then, the ethanolic solution was added to the correct amount of TIP particles and EtOH was slowly evaporated in the rotary evaporator over 4 h.
Formulation 6: A fast disintegrating sublingual tablet was manufactured using TIP particles. Therefore, harmine glucuronate was dissolved in dH2O to yield a concentration of 25% (m/v). The specific amount of TIP particles was calculated to yield a loading coefficient of the particles of 25%. Then, the aqueous solution was slowly dropped onto the powder (in a Petri dish) and constantly stirred to yield a homogeneous paste. The paste was then air-dried at room temperature overnight.

Further examples and/or embodiments are disclosed in the following numbered items.
1. A composition comprising harmine or a pharmaceutically acceptable salt thereof (preferably harmine) and
   (i) an uronic acid; or
   (ii) a carboxylic acid and a monosaccharide, preferably present in a molar ratio of between 0.5 and 2.0, more preferably present in a molar ration of about 1:1.
2. The composition of item 1, wherein harmine and the uronic acid in (i), or harmine and the carboxylic acid in (ii), are present in a molar ratio of between 0.5 and 2.0, preferably in a molar ratio of about 1:1.
3. The composition of item 1 or 2, wherein the composition comprises harmine and an uronic acid, preferably wherein the composition comprises a salt of harmine and uronic acid.
4. The composition of any one of items 1 to 3, wherein the composition is an amorphous composition or wherein the composition comprises a natural deep eutectic solvent.
5. A salt of harmine and uronic acid.
6. The composition of any one of items 1 to 4 or the salt of item 5, wherein the uronic acid is glucuronic acid or galacturonic acid.
7. The composition of item 1 or 2, wherein the carboxylic acid is malic acid or acetic acid, and/or wherein the monosaccharide is glucose or fructose.
8. A kit of parts comprising:
   (a) the composition of any one of items 1 to 4, 6 or 7 or the salt of item 5 or 6 and a pharmaceutically acceptable carrier; and
   (b) DMT or a pharmaceutically acceptable salt thereof and a pharmaceutically acceptable carrier.
9. A pharmaceutical composition comprising
   (a) the composition of any one of item 1 to 4, 6 or 7 and/or the salt of item 5 or 6; and
   (b) DMT or a pharmaceutically acceptable salt thereof;
   and a pharmaceutically acceptable carrier.
10. A pharmaceutical composition comprising
   (a) the composition of any one of items 1 to 4, 6 or 7 and/or the salt of item 5 or 6;
   and a pharmaceutically acceptable carrier.
11. The composition of any one of items 1 to 4, 6 or 7, the salt of item 5 or 6, the kit of parts of item 8 or the pharmaceutical composition of item 9 or 10 for use as a medicament.
12. The composition of any one of items 1 to 4, 6 or 7, the salt of item 5 or 6, the kit of parts of item 8 or the pharmaceutical composition of item 9 for use in the treatment and/or prevention of a psychiatric, psychosomatic or somatic disorder.
13. The composition for use, the salt for use, the kit of parts for use or the pharmaceutical composition for use of item 12, wherein the psychiatric disorder is selected from depression, stress-related affective disorder, major depressive disorder, dysthymia, treatment-resistant depression, burnout, anxiety, post-traumatic stress disorder, addiction, eating disorder, and obsessive-compulsive disorder.
14. The composition for use or the salt for use of item 12 or 13, wherein said composition or said salt is to be administered simultaneously, separately or sequentially with DMT or its pharmaceutically acceptable salt.
15. The composition for use or the salt for use of item 14, or the kit of parts for use or the pharmaceutical composition for use of item 12 or 13, wherein the ratio of harmine to DMT is between 0.5 to 2.0, preferably about 1.0.
16. The composition for use of item 14 or 15, the salt for use of item 14 or 15, the kit of parts for use of any one of items 12, 13 or 15, or the pharmaceutical composition for use of any one of items 12, 13 or 15,
   wherein harmine and DMT are to be administered incrementally,
   preferably wherein each increment of harmine is between 5 mg and 80 mg and/or each increment of DMT is between 5 mg and 50 mg, and/or wherein the total dose of harmine is between 100 mg and 300 mg and/or the total dose of DMT is between 50 mg and 150 mg, and/or wherein interval between the increments is between 5 and 60 minutes; or
   wherein harmine and DMT are to be administered as a single bolus dose,
   preferably wherein the total dose of harmine is between 5 mg and 200 mg and/or the total dose of DMT is between 5 mg and 100 mg.
17. The composition for use of any one of items 14 to 16, the salt for use of any one of items 14 to 16, the kit of parts for use of any one of items 12, 13, 15 or 16, or the pharmaceutical composition for use of any one of items 12, 13, 15 or 16, wherein harmine and/or DMT are to be administered sublingually.
18. The composition of any one of items 1 to 4, 6 or 7, the salt of item 5 or 6 or the pharmaceutical composition of item 10 for use in the treatment and/or prevention of a disease or disorder selected from Parkinson's disease, Alzheimer's disease and other types of dementias, stroke, multiple sclerosis, neurodegeneration/-inflammation, neuronal damage due to excessive substance abuse, autonomic dysfunction, pain syndromes, cardiovascular disorders, cancer, infectious diseases (preferably caused by fungi infection, helminth infection, or bacterial infection), diabetes, autoimmune disease, asthma, bronchitis, and arthritis.

1. A composition comprising harmine or a pharmaceutically acceptable salt thereof and
(i) a uronic acid; or
(ii) a carboxylic acid and a monosaccharide present in a molar ratio of between 0.5 and 2.0.

2. The composition of claim 1, wherein harmine and the uronic acid in (i), or harmine and the carboxylic acid in (ii), are present in a molar ratio of between 0.5 and 2.0.

3. The composition of claim 2, wherein harmine and the uronic acid in (i), or harmine and the carboxylic acid in (ii), are present in a molar ratio of about 1:1.

4. The composition of any one of claims 1 to 3, wherein the composition comprises harmine or a pharmaceutically acceptable salt thereof and (ii) an uronic acid.

5. The composition of claim 4, wherein the composition comprises a salt of harmine and uronic acid.

6. The composition of any one of claims 1 to 4, wherein harmine or a pharmaceutically acceptable salt thereof is harmine.

7. The composition of any one of claims 1 to 6, wherein the composition is an amorphous composition or wherein the composition comprises a natural deep eutectic solvent.

8. A salt of harmine and uronic acid.

9. The composition of any one of claims 1 to 7 or the salt of claim 8, wherein the uronic acid is glucuronic acid or galacturonic acid.

10. The composition of claim 9 or the salt of claim 9, wherein the uronic acid is glucuronic acid.

11. The composition of claim 1 or 2, wherein the composition comprises harmine or a pharmaceutically acceptable salt thereof and (ii) a carboxylic acid and a monosaccharide present in a molar ratio of between 0.5 and 2.0.

12. The composition of claim 11, wherein harmine or a pharmaceutically acceptable salt thereof and (ii) a carboxylic acid and a monosaccharide are present in a molar ratio of about 1:1.

13. The composition of claim 1, 2, 11 or 12, wherein the carboxylic acid in (ii) is malic acid or acetic acid, and/or wherein the monosaccharide in (ii) is glucose or fructose.

14. A kit of parts comprising:
(a) the composition of any one of claims 1 to 7, or 9 to 13 or the salt of any one of claims 8 to 10 and a pharmaceutically acceptable carrier; and
(b) DMT or a pharmaceutically acceptable salt thereof and a pharmaceutically acceptable carrier.

15. A pharmaceutical composition comprising
(a) the composition of any one of claims 1 to 7, or 9 to 13 and/or the salt of any one of claims 8 to 10; and
(b) DMT or a pharmaceutically acceptable salt thereof;
and a pharmaceutically acceptable carrier.

16. A pharmaceutical composition comprising
(a) the composition of any one of claims 1 to 7, or 9 to 13 and/or the salt of any one of claims 8 to 10;
and a pharmaceutically acceptable carrier.

17. The composition of any one of claims 1 to 7, or 9 to 13, the salt of any one of claims 8 to 10, the kit of parts of claim 14 or the pharmaceutical composition of claim 15 or 16, wherein the composition, the salt, the part(s) of said kit of parts or the pharmaceutical composition is/are formulated by using carrier particles with secondary internal structures, preferably wherein said carrier particles comprise hydroxyapatite.

18. The composition of any one of claim 1 to 7, 9 to 13 or 17, the salt of any one of claim 8 to 10, or 17, the kit of parts of claim 14 or 17 or the pharmaceutical composition of any one of claims 15 to 17 for use as a medicament.

19. The composition of any one of claim 1 to 7, 9 to 13 or 17, the salt of any one of claim 8 to 10 or 17, the kit of parts of claim 14 or 17 or the pharmaceutical composition of claim 15 or the pharmaceutical composition of claim 17 insofar dependent on claim 15, for use in the treatment and/or prevention of a psychiatric, psychosomatic or somatic disorder.

20. The composition for use, the salt for use, the kit of parts for use or the pharmaceutical composition for use of claim 19, wherein the psychiatric disorder is selected from depression, stress-related affective disorder, major depressive disorder, dysthymia, treatment-resistant depression, burnout, anxiety, post-traumatic stress disorder, addiction, eating disorder, and obsessive-compulsive disorder.

21. The composition for use, the salt for use, the kit of parts for use or the pharmaceutical composition for use of claim 19 or 20, wherein said composition or said salt is to be administered simultaneously, separately or sequentially with DMT or its pharmaceutically acceptable salt.

22. The composition for use, the salt for use, the kit of parts for use or the pharmaceutical composition for use of any one of claims 19 to 21, wherein preferably the ratio of harmine to DMT is between 0.5 to 2.0, preferably about 1.0.

23. The composition for use, the salt for use, the kit of parts for use or the pharmaceutical composition for use of any one of claims 19 to 22, wherein harmine and/or DMT are to be administered sublingually.

24. The composition for use, the salt for use, the kit of parts for use, or the pharmaceutical composition for use of any one of claims 19 to 23,
wherein harmine and DMT are to be administered incrementally,
preferably wherein each increment of harmine is between 5 mg and 80 mg and/or each increment of DMT is between 5 mg and 50 mg, and/or wherein the total dose of harmine is between 100 mg and 300 mg and/or the total dose of DMT is between 50 mg and 150 mg, and/or wherein interval between the increments is between 5 and 60 minutes.

25. The composition for use, the salt for use, the kit of parts for use or the pharmaceutical composition for use of any one of claims 19 to 23,
wherein harmine and DMT are to be administered as a single bolus dose,
preferably wherein the total dose of harmine is between 5 mg and 200 mg and/or the total dose of DMT is between 5 mg and 100 mg.

26. The kit of parts of claim 14, the pharmaceutical composition of claim 15, the kit of parts for use of claim 18, the pharmaceutical composition for use of claim 18 insofar dependent on claim 15 or the kit of parts or the pharmaceutical composition for use of any one of claims 19 to 25, wherein DMT or a pharmaceutically acceptable salt thereof is DMT hemisuccinate.

27. The composition of any one of claim 1 to 7, 9 to 13 or 17, the salt of any one of claim 8 to 10 or 17, or the pharmaceutical composition of claim 16 or the pharmaceutical composition of claim 17 insofar dependent on claim 16, for use in the treatment and/or prevention of a disease or disorder selected from Parkinson's disease, Alzheimer's disease and other types of dementias, stroke, multiple sclerosis, neurodegeneration/-inflammation, neuronal damage due to excessive substance abuse, autonomic dysfunction, pain syndromes, cardiovascular disorders, cancer, infectious diseases (preferably caused by fungi infection, helminth infection, or bacterial infection), diabetes, autoimmune disease, asthma, bronchitis, and arthritis.

28. A DMT hemisuccinate salt.

29. A crystal form A of the salt of claim 28, characterized by X-ray powder diffraction pattern (Cu-Kα₁) comprising a peak at about 16.14±0.2° (2θ).

30. The crystal form of claim 29, wherein the X-ray powder diffraction pattern (Cu-Kα₁) further comprises one or more peaks selected from 13.50±0.2°, 17.84±0.2°, 19.67±0.2°, 21.81±0.2°, 23.19±0.2°, and 25.36±0.2° (2θ).

31. A crystal form B of the salt of claim 30, characterized by the X-ray powder diffraction pattern (Cu-Kα₁) comprising a peak at about 15.57±0.2° (2θ).

32. The crystal form of claim 31, wherein the X-ray powder diffraction pattern (Cu-Kα₁) further comprises one or more peaks selected from 10.09±0.2°, 16.52±0.2°, 16.82±0.2°, 17.06±0.2°, 19.34±0.2°, 19.93±0.2°, 21.13±0.2°, 22.91±0.2°, and 23.45±0.2° (2θ).

33. A method for masking the bitterness of a compound,
wherein the compound is harmine or a pharmaceutically acceptable salt thereof, or DMT or a pharmaceutically acceptable salt thereof, the method comprising loading a compound having a bitter taste onto a carrier particle wherein
a) the carrier particle comprises a loading cavity and wherein the carrier particle comprises a basic salt; and
b) wherein the bitterness of the compound is masked by the carrier particle during oral mucosal absorption.

34. A pharmaceutical composition comprising carrier particles, comprising:
a) a carrier particle comprising a loading cavity and comprising of a basic salt; and
b) a compound having a bitter taste, wherein the compound is harmine or a pharmaceutically acceptable salt thereof, or DMT or a pharmaceutically acceptable salt thereof,
wherein the bitterness of the compound is masked by the carrier particle during oral mucosal absorption.

35. The method for masking the bitterness of a compound of claim 33 or the pharmaceutical composition comprising carrier particles of claim 34, wherein the carrier particle is obtained by the steps of:
a) combining a carrier material with a template material, wherein the carrier material forms a primary structure around the template material;
b) transforming the template material;
c) removing the transformed template material; and
d) obtaining carrier particles with secondary internal structures.

36. The method for masking the bitterness of a compound of claim 33 or 35 or the pharmaceutical composition comprising carrier particles of claim 34 or 35, wherein the template material is an inorganic material or consists primarily of inorganic material; and/or
wherein the carrier material is an inorganic material or consists primarily of inorganic material.

37. The method for masking the bitterness of a compound of claim 33, 35 or 36, or the pharmaceutical composition comprising carrier particles of any one of claims 34 to 36, wherein the carrier material and the template material are inorganic salts or consist primarily of inorganic salts.

38. The method for masking the bitterness of a compound of any one of claim 33 or 35 to 37 or the pharmaceutical composition comprising carrier particles of any one of claims 34 to 37, wherein combining a carrier material with a template material comprises chemical precipitation, layering and/or crystallization of the carrier material on the template material;
wherein removing the template material comprises dissolution of the transformed template material to form secondary internal structures; and/or
wherein transforming the template material comprises heating to a temperature from 600° C. to 1200° C., preferably
a) heating to a temperature from 600° C. to 900° C.;
b) wherein the step of transforming the template material comprises calcination; and/or
c) wherein the step of transforming the template material comprises a subsequent addition of water, preferably wherein the addition of water is an exothermic reaction.

39. The method for masking the bitterness of a compound of any one of claim 33 or 35 to 38 or the pharmaceutical composition comprising carrier particles of any one of claims 34 to 38, wherein the template material comprises calcium carbonate; and/or
wherein the carrier material comprises at least one salt and/or complex selected from the group of calcium phosphate and magnesium phosphate; preferably
a) wherein the carrier particles have a diameter of 1 to 300 μm;
b) wherein the carrier particles have a surface area between 15 m2/g to 400 m2/g;
c) wherein the secondary internal structure comprises pores having a diameter size in the range of ≥0.2 μm and ≤1.5 μm; and/or
d) wherein the total volume of the secondary internal structures in the obtained carrier particles with secondary internal structures is in the range of ≥10% to ≤90% of the particle volume.
